(19) United States
(12) Patent Application Publication
(10) Pub. No.: US 2014/0298494 A1
(43) Pub. Date: Oct. 2, 2014

(54) ANIMAL MODEL OF AUTISM

(71) Applicant: Matthew P. Anderson, Wellesley, MA (US)

(72) Inventor: Matthew P. Anderson, Wellesley, MA (US)

(73) Assignee: Beth Israel Deaconess Medical Center, Inc., Boston, MA (US)

(21) Appl. No.: 14/234,696

(22) PCT Filed: Jul. 23, 2012
(86) PCT No.: PCT/US2012/047839

§ 371 Date: Jun. 18, 2014

Related U.S. Application Data
(60) Provisional application No. 61/511,257, filed on Jul. 25, 2011.

(51) Int. Cl. A01K 67/027; C12Q 1/25; G01N 33/573; C12N 15/85

(52) U.S. Cl. CPC A01K 67/0278; C12N 15/8509; C12Q 1/25; G01N 33/573; C12N 2015/8536
USPC 800/3; 435/325; 435/366; 435/354; 800/9; 435/4; 435/7.4

(57) ABSTRACT

Some aspects of this invention provide a non-human animal model of autism. Some aspects of this invention provide a non-human animal model for diseases or disorders associated with an overexpression or a copy number variance of a Ube3a gene. Transgenic mammals and transgenic mammalian cells comprising an exogenous copy or exogenous copies of a ube3a protein-encoding nucleic acid sequence are also provided. Some aspects of this invention further provide methods for using the animal models, cells, and transgenic animals for identifying agents or interventions that can alleviate a pathogenic characteristic observed in the animal model, cell, or transgenic animal.

### RELATED APPLICATION

This application claims the benefit under 35 U.S.C. §119(e) of U.S. provisional patent application Ser. No. 61/511,257, filed Jul. 25, 2011, the contents of which is incorporated herein by reference in its entirety.

### GOVERNMENT SUPPORT

This invention was made with Government support under grants R21NS070295, R0I NS057444, K02 NS054674-03, awarded by the National Institute of Neurological Disorders and Stroke. The U.S. Government has certain rights in the invention.

### BACKGROUND OF THE INVENTION

The cellular and molecular basis of autism remains largely undefined, but recent studies uncovered genome copy number variations (CNVs) in idiopathic autism patients. Two common autism copy number variations, maternal 15q11-13 duplication (dup15) and triplication (isodicentric extranumerary chromosome, idic15), contain several candidate genes for the autism behavioral traits.

Autism spectrum disorders are estimated to affect 1 in 110 individuals and are behaviorally defined by three core traits: (i) impaired social interaction, (ii) reduced communication, and (iii) increased repetitive, stereotyped behaviors (1). Despite high heritability as evidenced by sibling, twin, and family studies (2), the diagnosis is based solely on behavioral criteria. Phenotypic heterogeneity and frequent medical co-morbidities also present significant challenges for animal modeling and translational research. Existing mouse models of syndromic neurodevelopmental disorders such as Rett Syndrome, Fragile X, and tuberous sclerosis have proven invaluable for investigations of these specific conditions, but the presence of multiple other neurologic and pathologic co-morbidities (e.g., mental retardation, tumors) and incomplete autism penetrance cause difficulties in making direct links between the autism-related behavioral defects and their neurobiological underpinnings. Animal models based on recently identified genetic defects in idiopathic, non-syndromic autism patients hold great promise in achieving this goal.

Recent advances in DNA array hybridization technologies have helped establish the presence of a high rate of small genomic DNA copy number variations (CNVs) in autism, present in 10-20% of cases (3-7). The altered gene dosages resulting from these CNVs may explain a significant proportion of autism cases. Maternally-inherited 15q11-13 duplications and triplications are amongst the most common genomic copy number variations found in autism (1-3%) (3, 8). Autism traits are found in about 50% of individuals with one extra maternal 15q11-13 copy resulting from an inverted duplication (dup15), while near complete autism penetrance is observed in individuals with two extra maternal copies resulting from an isodicentric extranumerary chromosome (idic15)(8). Importantly, paternally inherited duplications (with rare exceptions) typically do not associated with autism (8). The observations suggested the dosage of an imprinted gene or genes within the duplicated region underlies the autism risk in these patients.

### SUMMARY OF THE INVENTION

Some aspects of this invention relate to the surprising discovery that increased E3 ubiquitin-protein ligase, Ube3a (also known as E6-AP) gene copy number underlies autism in idic15 subjects and causes glutamatergic circuit defects. Some aspects of this invention relate to the recognition that Ube3a is the only gene within the 15q11-13 duplicated segment consistently shown to express solely from the maternal allele in brain (9), making it a likely candidate to mediate the autism phenotype. Furthermore, mutations or deletions causing Ube3a deficiencies underlie Angelman syndrome, a neurological disorder characterized by mental retardation, hypotonia and seizures (10, 11). The imprinting pattern is preserved in mice and the inheritance of a maternal allele deletion is sufficient to reconstitute many of the features of Angelman syndrome including seizures, defective motor performance, impaired contextual fear learning, defective synaptic long-term potentiation, and decreased dendritic spines (10, 12, 13). The cellular mechanism by which Ube3a deficiency in Angelman syndrome causes cognitive impairments is not fully understood. However, the Angelman mouse model displays a significant increase in the phosphorylation of hippocampal alpha calcium/calmodulin-dependent protein kinase II (αCaMKII), specifically at sites Thr(286) and Thr(305) (14). Furthermore, by crossing the Angelman mouse model to mice with a αCaMKII-T305V/T306A mutant knock-in that prevents inhibitory auto-phosphorylation of αCaMKII, the incidence of seizures was decreased, and the defects in motor function, learning, and synaptic plasticity were partially reversed (15). More recently, the Angelman mouse model was shown to display impaired experience-dependent maturation of visual cortex and experience-dependent defects in synaptic plasticity which was rescued by dark-rearing the animals (16, 17). However, the proteins ubiquitinated by Ube3a to mediate these behavioral and synaptic defects have not been identified.

Ube3a (E6-AP) was originally discovered to ubiquitinate and promote degradation of p53, playing a pathogenic role in human papilloma virus induced cervical epithelium neoplasia (18). More recently, Ube3a was shown to ubiquitinate and promote degradation of two important neuronal proteins, Arc and Ephexin5 (19, 20).

Some aspects of this invention relate to the surprising discovery that non-human mammals, for example, mice, carrying one or more extra gene copies of the ubiquitin protein ligase Ube3a, phenocopy three core autism-related behavioral traits: (i) defective social interaction, (ii) impaired adult ultrasonic vocalizations, and (iii) increased repetitive grooming behavior. Some aspects of this invention relate to the discovery that the occurrence and severity of the autism traits in mice carrying extra copies Ube3a depends on Ube3a copy number. Further, it was discovered that glutamatergic, but not GABAergic synaptic transmission is suppressed in such mammal, for example, in the idic15 mouse model, as a result of increased Ube3a copy number. The glutamate synapse defect results from both presynaptic and postsynaptic effects with reduced presynaptic release probability, synaptic glutamate concentration, and postsynaptic action potential coupling. These discoveries establish Ube3a dosage as a critical factor underlying the autism traits in idic15 patients and identify specific functional defects in glutamatergic synaptic transmission that may underlie this human behavioral disorder.

Some aspects of this invention relate to the recognition that an increased Ube3a gene copy number reconstitutes the autism behavioral traits found in dup15 and idic15 patients. As autism behavioral traits are weakly penetrant in dup15 patients, but highly penetrant in idic15 patients (8), autism-like traits were compared in mice expressing a two or three-fold excess of ube3a protein, modeling dup15 and idic15, respectively. Accordingly, some aspects of this invention provide a transgenic non-human mammal, for example, a mouse, that expresses an increased amount of ube3a protein, for example, as a result of an increased Ube3a copy number. Such transgenic mammals are useful, for example, as models of autism disorder and provide insights into the neural circuit pathogenesis of the disease. For example, some aspects of this invention provide a ube3a-idic15 mouse model, which displays correlates of all three diagnostic autism traits.

Some aspects of this invention provide an isolated transgenic mammalian cell comprising one or more isolated nucleic acid sequence(s) encoding a ubiquitin ligase 3a (ube3a) protein. In some embodiments, an isolated transgenic mammalian cell comprising one or more exogenous nucleic acid sequence(s) encoding a ube3a protein is provided. In some embodiments, an isolated transgenic mammalian cell comprising one or more recombinant nucleic acid sequence(s) encoding a ube3a protein is provided. In some embodiments, an isolated transgenic mammalian cell comprising one or more nucleic acid to sequence(s) encoding a ube3a protein in addition to any endogenous copies of nucleic acid sequences encoding a ube3a protein is provided. In some embodiments, the nucleic acid sequence(s) encoding a ube3a protein are stably integrated into the genome of the cell. In some embodiments, the cell comprises one isolated, exogenous, recombinant, or additional nucleic acid sequence encoding ube3a. In some embodiments, the cell comprises two isolated, exogenous, recombinant, or additional nucleic acid sequences encoding ube3a. In some embodiments, the genome of the cell further comprises one or more endogenous nucleic acid sequence(s) encoding a ube3a protein. In some embodiments, the genome of the cell comprises one or two endogenous nucleic acid sequence(s) encoding a ube3a protein. In some embodiments, the genome of the transgenic mammal comprises three endogenous nucleic acid sequences encoding a ube3a protein. In some embodiments, the genome of the transgenic mammal comprises an idic15 mutation. In some embodiments, the cell is a human cell. In some embodiments, the cell is a non-human mammalian cell. In some embodiments, the cell is a mouse cell. In some embodiments, the cell is derived from a mouse of FVB, dup15 or idic15 genetic background. In some embodiments, the cell is a neuronal cell. In some embodiments, the cell is an embryonic stem cell. In some embodiments, the one or more isolated nucleic acid sequence(s) encoding a ube3a protein comprise a ube3a cDNA. In some embodiments, the one or more isolated nucleic acid sequence(s) encoding a ube3a protein comprise a ube3a-encoding genomic region. In some embodiments, the one or more isolated nucleic acid sequence(s) encoding a ube3a protein comprise an isolated genomic fragment comprising a wild-type ube3a coding sequence. In some embodiments, the one or more isolated nucleic acid sequence(s) encoding a ube3a protein comprise a wild-type ube3a coding sequence and/or ube3a gene. In some embodiments, the wild-type ube3a coding sequence or ube3 gene is a human or a mouse ube3 coding sequence or gene. In some embodiments, the one or more isolated nucleic acid sequence(s) encoding a ube3a protein comprise a fragment of mouse chromosome 7. In some embodiments, the fragment is approximately 162 kb long. In some embodiments, the fragment comprises the exon-intron coding sequence of ube3a. In some embodiments, the fragment is about 78 kb long. In some embodiments, the fragment comprises at least about 1 kb, at least about 2 kb, at least about 3 kb, at least about 4 kb, at least about 5 kb, at least about 10 kb, at least about 20 kb, at least about 25 kb, at least about 30 kb, at least about 40 kb, at least about 50 kb, at least about 60 kb, at least about 70 kb, at least about 80 kb, at least about 90 kb, or at least about 100 kb of the chromosome 7 region immediately upstream (5′) of the exon-intron coding sequence of ube3a. In some embodiments, the fragment comprises about 63 kb of the chromosome 7 region immediately upstream (5′) of the exon-intron coding sequence of ube3a. In some embodiments, the fragment comprises at least about 1 kb, at least about 2 kb, at least about 3 kb, at least about 4 kb, at least about 5 kb, at least about 10 kb, at least about 20 kb, at least about 25 kb, at least about 30 kb, at least about 40 kb, at least about 50 kb, at least about 60 kb, at least about 70 kb, at least about 80 kb, at least about 90 kb, or at least about 100 kb of the chromosome 7 region immediately downstream (3′) of the exon-intron coding sequence of ube3a. In some embodiments, the fragment comprises at about 21 kb of the chromosome 7 region immediately downstream (3′) of the exon-intron coding sequence of ube3a. In some embodiments, the one or more isolated nucleic acid sequence(s) encoding a ube3a protein further comprises a sequence encoding a tag. In some embodiments, the tag is in-frame with the open reading frame of ube3a and encodes a tagged ube3a fusion protein. In some embodiments, the tag is a FLAG tag. In some embodiments, the one or more isolated nucleic acid sequence(s) encoding a ube3a protein comprises a wild type ube3a promoter. In some embodiments, the one or more isolated nucleic acid sequence(s) encoding a ube3a protein comprises a heterologous promoter. In some embodiments, the heterologous promoter is a constitutive promoter. In some embodiments, the heterologous promoter is a cell-type specific promoter or a tissue specific promoter. In some embodiments, the promoter is active in neuronal cells or tissues. In some embodiments, the heterologous promoter is an inducible promoter. In some embodiments, the inducible promoter is a drug-inducible promoter. In some embodiments, the inducible promoter is a recombination-inducible promoter. In some embodiments, the inducible promoter is active after cre-recombinase-mediated recombination. In some embodiments, the cell further comprises an expression construct comprising a nucleic acid encoding cre recombinase under the control of a cell-type specific promoter. In some embodiments, the cell-type specific promoter is a neuronal cell type specific promoter. In some embodiments, the cell is comprised in a non-human mammal.

Some aspects of this invention provide a non-human mammal comprising at least one ube3a transgenic cell as described herein. In some embodiments, a non-human mammal comprising at least one ube3a transgenic cell as described herein within its germ line, e.g. a ube3a transgenic germ cell, is provided. In some embodiments, a non-human mammal consisting of ube3a transgenic cells as described herein is provided. In some embodiments, the non-human mammal is a mouse. In some embodiments, the mouse exhibits one or more of (i) impaired social interaction; (ii) defective communication (e.g., vocalization); and/or (iii) repetitive behavior (e.g., self-grooming). In some embodiments, a non-human mammal is provided that comprises at least one expression construct comprising a nucleic acid sequence encoding a ube3a protein stably integrated into the genome of at least one cell comprised in the non-human mammal.

Some aspects of this invention provide methods of use of any of the ube3a transgenic cells or non-human ube3a-transgenic mammals described herein. In some embodiments, the methods of use comprise the use of the cells or mammals as a model for: (a) studying the molecular mechanisms of, or physiological processes associated with autism; (b) identification and/or testing of an agent useful in the prevention, amelioration or treatment of autism; (c) identification of a protein and/or nucleic acid diagnostic marker for autism; and/or (d) studying the molecular mechanisms of, or physiological processes or medical conditions associated with increased copy number of a ube3a-encoding nucleic acid, and/or with undesirable activity, expression, or production of ube3a.

Some aspects of this invention provide a method of identifying an agent for the treatment of a symptom associated with autism, the method comprising administering a candidate agent to a transgenic non-human mammal comprising an isolated, exogenous, or additional ube3a protein-encoding nucleic acid sequence or expressing an elevated level of ube3a protein, and exhibiting or expected to develop at least one symptom associated with autism. In some embodiments, the method further comprises determining whether the administration of the candidate agent effected an amelioration of the symptom: In some embodiments, if the administration of the candidate agent effected an amelioration of the symptom, then the candidate agent is identified as an agent for the treatment of a symptom associated with autism. In some embodiments, the symptom associated with autism is (i) impaired social interaction, (ii) reduced communication, and/or (iii) increased repetitive, stereotyped behavior, (iv) reduced or impaired glutamatergic synaptic transmission, (v) reduced/impaired presynaptic glutamate release, and/or (vi) reduced/impaired postsynaptic excitability to phasic synapse-like stimuli.

Some aspects of this invention provide a method of identifying an agent for the treatment of a pathological characteristic associated with autism. In some embodiments, the method comprises contacting a candidate agent with a transgenic cell comprising an isolated, exogenous, or additional ube3a protein-encoding nucleic acid sequence or expressing an elevated level of ube3a protein, and exhibiting or expected to develop at least one pathological characteristic associated with autism. In some embodiments, the method further comprises determining whether the candidate agent effected an amelioration of the pathological characteristic in the cell. In some embodiments, if an amelioration of the pathological characteristic is observed as a result of the contacting, then the candidate agent is identified as an agent for the treatment of a pathological characteristic associated with autism. In some embodiments, the pathological characteristic is selected from the group consisting of reduced or impaired glutamatergic synaptic transmission, reduced/impaired presynaptic glutamate release, and reduced/impaired postsynaptic excitability to phasic synapse-like stimuli.

Some aspects of this invention provide a method of identifying a diagnostic marker for autism. In some embodiments, the method comprises assessing the expression level of a biomolecule in a cell, tissue, or sample of a transgenic non-human mammal comprising an increased ube3a protein-encoding nucleic acids copy number and comparing the expression level to a control or reference level. In some embodiments, if the biomolecule expression level in the transgenic mammal is different from the control level, then differential expression of the biomolecule is identified as a diagnostic biomarker for autism. In some embodiments, the biomolecule is a protein or a nucleic acid. In some embodiments, the control level representative of the level of expression the biomolecule in a healthy mammal of the same species.

In some embodiments, a method of diagnosing an increased risk of developing autism or an autism spectrum disorder in a subject is provided. In some embodiments, the method comprises determining a level of a ube3a protein in a sample obtained from the subject and comparing the level of ube3a determined in the subject to a control or reference level. In some embodiments, if the level of ube3a protein detected in the subject is higher than the control or reference level, the subject is identified as a subject at an increased risk of developing autism or an autism spectrum disorder. In some embodiments, the control or reference level is a level of ube3a protein representative of a sample obtained from a subject not at an increased risk of developing autism or an autism spectrum disorder. In some embodiments, the control or reference level is a level of ube3a representative of a sample obtained from a healthy subject.

The above summary provides an overview over some non-limiting aspects of this invention. Additional aspects, embodiments, advantages, features, and uses of the invention will become apparent from the following detailed description of non-limiting embodiments of the invention when considered in conjunction with the accompanying drawings.

### BRIEF DESCRIPTION OF THE DRAWINGS

The application file contains at least one drawing executed in color.

FIG. 1. Ube3a Gene Copies Added to Model 15q11-13 Duplication Autism.

(A) Recombineering a c-terminal FLAG-tag into a wild-type Ube3a gene (162 kb, bacterial artificial chromosome, BAC vector) inserted at the 3′ coding/untranslated boundary of exon 12 in frame with the C-terminus followed by two translational stop codons. The nucleotide sequence shown is SEQ ID NO:13; the amino acid sequence shown is SEQ ID NO:14. (B) Schematic representation of the genes located between breakpoint (BP) 1 and BP3 in the 15q11-13 region. Paternally-expressed genes are blue (MKRN3, MAGEL2, NDN, SNURF/SNRPN), maternally-expressed genes are red (UBE3A), and the location of the genomic DNA contained in the BAC is green (RP24-178G7). (C) Quantification of Ube3a protein in maternal Ube3a knockout (KO), wild-type (WT), 1×Tg, and 2×Tg Ube3a transgenic mice (total brain protein, Ube3a antibody). (ANOVA: F_{(3,24)}=26.95, *P<0.05 **P<0.001 by Dunnett's post-hoc n=4-11). (D) Double immunofluorescence staining for total Ube3a (red) and Ube3a-FLAG transgene (green) reveals complete overlap of native and transgenic protein.

FIG. 2. Ube3a Gene Dosage Effects on Social Behavior

(A) Diagram of three chamber social interaction test with choice between a novel container containing a novel mouse or a novel empty container. (B) Wild-type mice, but not 1×Tg and 2×Tg mice, show a significant preference for the social third (*P=0.0162 comparing within-genotype “Social” and “Opposite” by t test). (C) Time spent interacting with either the caged mouse (social) or the novel container (opposite). (*P=0.0157 and 0.0186 respectively, t test). 2×Tg mice showed no social preference (P<0.05, t test). N_{(Wt,} 1×, 2×)=11, 15, 12. (D) Diagram of modified three-chambered social interaction test with choice to explore or not explore the novel mouse. (E) Time in the social side of the enclosure. *P<0.002, t test. (F) Time in area proximal to the enclosures (dark circles, “Close” zone). *P<0.005, t test. N_{(Wt,} 1×, 2×)=17, 10, 15. (G) A caged object rather than the novel mouse to test for novel object exploration. *P<0.03, t test, comparing within-genotype “Object” and “Opposite”; n=11-13. (H) Independent Ube3a transgenic founder lines 1 (Fd1) and 2 (Fd2) (n=10, 5) display decreased social preference compared to wild-type littermates (n=7, 4). ****P<0.001, ** P<0.01, t test. Mean±S.E.M. are plotted. Color code: wild-type (black, left column group in B, C, E, F, and G, first and third column group in H), single Ube3a transgenic (1×, blue, middle column group in B, C, E, and F), and double Ube3a transgenic (2×, red, right columns group in B, C, E, F, and G, second and fourth column group in H).

FIG. 3. Ube3a Gene Dosage Effects on Ultrasonic Communicative Vocalizations and Self Grooming

(A) Number of social ultrasonic vocalizations made by pairs of genotype- and sex-matched mice. (Kruskal-Wallis test: H=7.76, df=2, P=0.021, *P<0.05 by Dunn's multiple comparison post-hoc, n=8-14). (B) Ultrasonic vocalization responses of male mice to female urine measuring number (ANOVA: F_{(2,22)}=4.52, P=0.023, *P<0.05 by Dunnett's post-hoc) and duration (ANOVA: F_{(2,22)}=5.31, P=0.013, *P<0.05 by Dunnett's post-hoc). n=7-11. (C) and (D) Representative examples and distribution of vocalization types in urine-exposed males defined by shape and harmonics. ns, not significant by ANOVA. Left columns (black): wild type, right columns (red): 2×Tg. (E) Olfactory habituation/dishabituation test showing no significant effect of genotype (2-way ANOVA F_{(1,132)}=2.723, P=0.12, n=7). (F) Ultrasonic vocalizations of pups during acute maternal separation (ANOVA: F_{(2,262)}=0.87, P=0.42, n=10-14). G) Repetitive self-grooming (ANOVA: F_{(2,34)}=5.41, P=0.0095, **P<0.01 by Dunnett's post-hoc, n=11-12). (H) Increased grooming in independent Ube3a transgenic founder lines 1 (Fd1, N_{wt,2×}=10, 14) and 2 (Fd2) (N_{wt,2×}=3,5). *P=0.01, **P=0.004, t test. Mean±S.E.M. are plotted. Color code: wild-type (WT, black,), single-transgenic Ube3a (1×, blue), double-transgenic Ube3a (2×, red) mice. N refers to number of mice.

FIG. 4. Ube3a Gene Dosage Effects on Excitatory and Inhibitory Synaptic Transmission

(A) Evoked EPSC (unbalanced repeat measures ANOVA F_{(1,97)}=41.45, **P<0.001 by Bonferroni post-hoc, n=6-8). (B) Evoked IPSC (unbalanced repeat measures ANOVA F_{(1,83)}=0.03, P=0.8551, n=6). (C) mEPSC traces (top) and cumulative frequency (CF) plots of amplitude (bottom, left) and interevent interval (bottom, right) (K-S Test, **P<0.01,*** P<0.001, n=9-11). Median mEPSC amplitude (inset left) and frequency (inset right). (ANOVA: amplitude P<0.01, frequency, P<0.01, t test n=9-11). (D) Miniature inhibitory postsynaptic current (mIPSC) traces (top) and cumulative amplitude (bottom, left) and frequency (bottom, right) plots (P>0.05, K-S test, n=9-11). Median mIPSC amplitude (inset left) and frequency (inset right) were similar across genotypes (P>0.05, t test). Mean+S.E.M. are plotted. Color code: wild-type (black) and double Ube3a transgenic (2×, red) mice. n refers to number of cells.

FIG. 5. Ube3a Gene Dosage Effects on Glutamate Synapse Number and Postsynaptic Glutamate Receptor Currents

(A) Synapse number, electron microscopy (P=0.67, t test n=3 animals per group, 28-32 micrographs per animal). (B) Co-immunostaining of pre-(vglut1) and post-(PSD95) synaptic markers (scale bar=1 μm. P=0.8706, t test, n=4 animals per group, ≧8 micrographs per animal). (C) Spine number per dendrite length, golgi staining (Apical P=0.64; Basal P=0.77, t test, n=4 animals per group, >10 dendrites per animal). (D) Glutamate ionophoresis induced AMPA and NMDA currents (AMPA: P=0.46, t test, n=5-6; NMDA: P=0.97, t test, n=3-6). (E) Fiber stimulation evoked AMPA/NMDA ratio (P=0.54, t test, n=6-8). Mean+S.E.M. are plotted. Color code: wild-type (black, left columns), and double Ube3a transgenic (2×, red, right columns) mice. n refers to number of mice (A-C) or cells (D and E).

FIG. 6. Ube3a Gene Dosage Effects on Release Probability, Synaptic Glutamate Concentration, and ES Coupling

(A) Representative paired-pulse traces in transgenic animals (right) and scaled to match first pulse to wild-type (left), and bar graph (P=0.028, t test, n=7-10). (B) Representative traces of unscaled (above) and scaled to wild-type (below) evoked EPSCs with (dotted lines) or without γDGG (solid lines) and graph (right) showing reduced EPSC amplitude and increased γDGG inhibition in Ube3a transgenic mice. P=0.0127, t test, n=7-8). (C) Representative voltage tracings (left) and graph (right) assessing firing response to a 5 ms EPSC-like somatic current (0-640 pA, 40 pA steps). (P=0.003, chi-square, n=14-17). Color code: wild-type (black) and double Ube3a transgenic (2×, red) mice. n refers to number of cells.

FIG. 7. Expression of Ube3a BAC transgene.

(a) Gel and graph showing Ube3a transgene copy number by semi-quantitative PCR in single- and double-transgenic mice (1×Tg and 2×Tg, Ube3a-L form). n=3-5, *P<0.001 by ANOVA with Dunnett's post-hoc. (b) Ube3a transgene is expressed independent of sex or parent-of-origin. Anti-FLAG western blot reveals the transgenic protein is expressed in both males and females, whether inherited from the father (M^{Wt}P^{Tg}) or the mother (M^{Tg}P^{Wt}). (c) The level of anti-FLAG immunoreactivity on western blots as in (b) was quantified from 4 animals per group. Two-way ANOVA reveals no difference in band intensity based on sex (F_{(1,12)}=0.01186, p=0.9151) or transgene parent-of-origin (F_{(1,12)=}0.6786, p=0.4261), and no interaction (F_{(1,12)}=1.007, p=0.3354). Mean±S.E.M. are plotted.

FIG. 8. Transgenic and native Ube3a proteins display similar patterns of expression in brain.

Brain slice patch-clamp recordings focused on pyramidal in layer 2/3 barrel cortex which strongly express native Ube3a in wildtype animals (a) and FLAG-tagged Ube3a in transgenic animals (b). Identical patterns of Ube3a staining are found in wild-type (anti-Ube3a, red) and transgenic (anti-FLAG, green) mouse thalamus (c, f), CA1 hippocampus (d, g) and cerebellar Purkinje cells (e, h). Note the absence of transgene and Ube3a in the cerebellar granule cell soma in both. Ube3a is concentrated in the nucleus (i, j, layer V pyramidal neurons). In 7DIV cortical neuron cultures from wildtype (km) or transgenic (np) mice, Ube3a localizes to PSD95-positive synapses (Green, anti Ube3a (k) or anti-FLAG (n); Red, anti PSD95 (l, o)). Scale bars 100 μm (a-h), 10 μm (i-j) 30 μm (k-p). The results closely match those of Gustin et al. (2010).

FIG. 9. Double Immunofluorescence staining for Ube3a and FLAG reveals complete overlap of the transgenic protein.

(a) Native Ube3a (Red) and transgenic Ube3a-FLAG (green) stained in transgenic animals demonstrates that FLAG is present exclusively and completely in 100% of Ube3a-positive cells. Higher-magnification views of upper barrel cortex (b) and the CA3 region of the hippocampus (c) reveal that all labeled neurons display a yellow color indicating overlap of the two markers. Increased staining intensity in the neuronal soma is observed for both transgenic and native Ube3a. Scale bar 500 μm (a), 100 μm (b, c). The results closely match those of Gustin et al. (2010).

FIG. 10. Social interaction is suppressed and grooming is increase by increased Ube3a (2×) gene dosage and they display no gender-specific effects.

(a) Male and female wild-type mice (black, n=5, 6) show a significant preference for the social zone (within-genotype T-test, ***p<0.005) in the three chamber social interaction paradigm. Neither male nor female double transgenic Ube3a mice (red, n=7, 8) show a preference for the social zone. (b) Both male and female double transgenic Ube3a-L mice show increased grooming. Two-way ANOVA of time spent grooming with gender and genotype as dependent variables shows a significant effect of genotype (F(3,38)=3.61, **P=0.0218), with no effect of gender (F(1,38)=0.03, P=0.8716), and no interaction (F(3,38)=0.35 P=0.7881). N (left to right bars)=7, 5, 7, 4, 5. Mean±S.E.M. are plotted. Color code: wild-type (black, left columns) and double-Ube3a transgenic (red, right column).

FIG. 11. Anxiety-like behavior and short-term memory are unaffected by Ube3a (2×) transgene.

Mice were placed in a 50 cm×100 cm plastic box in a brightly-lit room and their movement was recorded for 10 minutes. Both wild-type and double-transgenic mice moved a similar total distance (a), made similar numbers of entries into the center of the open field (b), and spent similar amount of time in the center of the open field (c), indicating a lack of generalized anxiety. (d-f) Anxiety-like behavior was tested in the elevated plus maze. (d) Both groups made similar entries into the open arms. (e) The fraction of entries into the open arms (open arms/(open+closed arms)) was similar. (f) The fraction of time spent in the open arms was similar. The results suggest a lack of generalized anxiety (NS=not significant). (g-i) Mice were tested in a short-term memory paradigm, diagrammed in (g). Mice were first allowed to explore two different objects for five minutes. (h) Both groups of mice made similar numbers of sniffs to both objects indicating normal object exploration. After a 15-minute break, the target object was exchanged for a novel object. (i) Both wild-type and 2×Tg mice showed a significant preference for the novel object (*within-genotype t test, P<0.05). N (wt, 2×Tg)=11, 13 for elevated plus; and 20, 21 for object memory. Mean+S.E.M. are plotted. For complete statistics see Table 1. Color code: wild-type (black, left columns) and double Ube3a transgenic (red, right columns).

FIG. 12. Developmental milestones and motor functions are normal in transgenic mice.

(a) Weight and weight gain is normal. (b) Time to roll over from their back was similar across genotypes. (c) Time to orient with its head up-hill when placed head-down on an inclined plane was similar across genotypes. N (Wt, 1×Tg, 2×Tg)=16, 24, 15 for all pup tests. (d) Rotorod performance in adult mice was similar. All mice improved over the three days of testing and there were no significant differences between the groups. N (wt, 1×Tg, 2×Tg)=31, 14, 20. Color code: wild-type (black, solid square), single—(1×, blue, upward pointing triangle) and double—(2×, red, downward pointing triangle) Ube3a transgenic. Mean±S.E.M. are plotted.

FIG. 13. Effects of gender on social vocalizations.

Ultrasonic vocalization responses of male and female mouse pairs (sex and genotype matched) were measured. (a) Two-way ANOVA of number of vocalizations with gender and genotype as dependent variables reveals a significant effect of both gender (F(1,49)=7.81, P=0.0074) and genotype (F(2,49)=3.32, P=0.0445) and a significant gender×genotype interaction (F(2,49)=3.80, P=0.0291). *P<0.05 wt vs. 2× females by Bonferroni post-hoc test; other comparisons (Wt vs. 1×Tg males, Wt vs. 1×Tg females) were not significant. (b) Two-way ANOVA of time spent vocalizing with gender and genotype as dependent variables also shows a significant effect of gender (F(1,49)=7.38 P=0.0091), but no significant effect of genotype (F(2,49)=2.61 P=0.0841) and no genotype×gender interaction (F(2,49)=3.17 P=0.0505). While there appears to be a trend towards lower numbers of vocalizations and less time spent vocalizing in both males and females, males vocalized so infrequently the differences are not significant. Mean±S.E.M. are plotted. Color code: wild-type (black), single—(1×, blue) and double—(2×, red) Ube3a transgenic.

FIG. 14. Spontaneous EPSCs and IPSCs from wildtype and Ube3a transgenic pyramidal neurons from layer 2/3 barrel cortex.

(a) Spontaneous excitatory postsynaptic current (sEPSC) traces (top) and cumulative frequency (CF) plots of amplitude (bottom left) and frequency (bottom right) show decreased amplitude and frequency of sEPSCs (*P<0.05, ***P<0.001; K-S test, n=4-8). (b) Spontaneous inhibitory postsynaptic current (sIPSC) traces (top) and cumulative amplitude (bottom left) and frequency (bottom right) plots (*P<0.05, K-S Test, n=4-8). (c) Miniature excitatory postsynaptic current (mEPSC) traces recorded at −80 mV. Cumulative frequency (CF) plots of amplitude (bottom left) and frequency (bottom right) show decreased amplitude and frequency of mEPSCs (KS test: amplitude, P<0.0001; frequency, P<0.0001). The mean amplitude and frequency were also significantly reduced (amplitude, P=0.0032; frequency, P=0.0136, t test, n=11, 11). Mean±S.E.M. are plotted. Color code: wild-type (black) and double (Ube3a 2×Tg, red) Ube3a transgenic mice.

FIG. 15. Reduced release probability, but similar readily releasable pool size and AMPA and NMDA kinetics in wild type and transgenic mice.

(a) Representative traces showing the response to 20 Hz minimal stimulation, 200 μm from the cell body. (b) Cumulative amplitude graph showing the magnitude of the cumulative amplitude is decreased in Ube3a (2×) transgenic (WT vs. 2×, P<0.0001, t test, n=10, 9), but readily releasable pool size, defined as the y-intercept of the linear portion of the curve, is not different (P=0.224, t test). (c) The number of vesicles in a single readily releasable pool, estimated as readily releasable pool size divided by quantal size, is also not different (P=0.405, n=10, 9). (d) The release probability, calculated as mean EPSC amplitude (the mean value of the 1st EPSCs, cumulative plot (b) divided by readily releasable pool size, is significantly reduced in Ube3a (2×) transgenic mice (P=0.0206, t test, n=10, 8). (e,f) Averaged AMPA (e) and NMDA (f) traces from representative cells (˜200 events per trace). When scaled so that the amplitude equals wild-type (right), the kinetics are similar. Decay time constants (graph) are also similar (t test: AMPA, P=0.715, n=11, 11; NMDA, P=0.669, n=8, 5). n refers to number of cells. Mean±S.E.M. are plotted. Color code: wild-type (black) and double (2×, red) Ube3a transgenic.

FIG. 16. Protein levels of potential Ube3a targets in wild-type vs. Ube3a(2×) transgenic barrel cortex.

Singly housed male mice were exposed to a novel object for three hours before sacrifice (as in Greer et al. 2010), and protein expression in the barrel cortex was assayed by western blot. *P=0.03, two-tailed, unpaired T-test, n=10-12. All unmarked comparisons not significant (P>0.05, n=4-12). Mean±S.E.M. are plotted. Color code: wild-type (black) and double (2×, red) Ube3a transgenic.

FIG. 17. Ube3a (2×) transgene fails to alter total amount of proteins regulating synaptic glutamate concentration.

Single housed male mice were exposed to a novel object for three hours before sacrifice (as in Greer et al. 2010), and protein expression in the barrel cortex was assayed by western blot. All unmarked comparisons not significant by t-test (P>0:05, n=8). Mean±S.E.M. are plotted. Color code: wild-type (black) and double (2×, red) Ube3a transgenic.

FIG. 18. Biophysical properties of wild-type and Ube3a transgenic pyramidal neurons of layer 2/3 barrel cortex.

(a) Representative voltage traces and graph showing firing frequency in response to 1 s current pulses of 0-280 pA in 15 pA steps. Repeated-measures ANOVA revealed no differences between genotypes (P>0.05; n=8-10). (b,c) Representative traces and graph showing firing response to a ramped current injection. ANOVA reveals no difference in the threshold to fire (P>0.05, t test, n=9-17). (d,e) Capacitance and resting membrane potential were also similar between groups (N.S., P>0.05, t test, n=9-17). Mean+S.E.M. are plotted. Color code: wild-type (black) and double (2×, red) Ube3a transgenic.

### DETAILED DESCRIPTION OF CERTAIN EMBODIMENTS

Autism is a disorder characterized by impaired social interaction and communication, and by restricted and repetitive behavior. Typically, the symptoms of autism begin to manifest in human subjects having autism before a child is three years old. The term autism, as used herein, refers to the disorder of autism itself, and to any other disease or disorder within the autism spectrum, also referred to herein as autism spectrum disorder, such as Asperger syndrome, characterized by delays in cognitive development and language, and Pervasive Developmental Disorder-Not Otherwise Specified (commonly abbreviated as PDD-NOS), which is typically diagnosed when some symptoms of autism are observed in a subject, but the full set of criteria for autism or Asperger syndrome are not met. Worldwide, about 1-2 per 1,000 children are diagnosed with autism, whereas in the US the number of diagnosed children is about 9 per 1,000. Autism is hereditary, and some mutations associated with autism have been identified, e.g., dup15 and idic15, both involving the duplication or triplication, respectively, of a large genomic region on chromosome 15 containing numerous genes. So far, the molecular mechanism underlying the development of autism has not been elucidated and individual gene contributions to the disorder are poorly understood. This lack of understanding of the genetic and environmental factors contributing to the disease hampers the development of pre-onset diagnostics and targeted therapeutics. A suitable animal model of autism would be highly desirable to investigate molecular and cellular pathologies associated with the disorder and to develop better diagnostic methods and therapeutics. So far, such an animal model has not been available.

Some aspects of this invention are based on the surprising discovery that mice carrying an increased copy number of a single gene within the dup15 or idic15 region, the Ube3a gene, encoding a ubiquitin ligase, phenocopy three characteristics of autism: (i) impaired social interaction, (ii) reduced communication (vocalization), and (iii) increased repetitive, stereotyped behaviors (grooming). Accordingly, some aspects of this invention provide that transgenic animals comprising additional copy numbers of a ube3a gene within the genome of some or all of their cells, or expressing an increased amount of ube3a protein, constitute a valuable model for autism.

Some aspects of this invention provide an animal model for autism. In some embodiments, the animal is a non-human mammal, for example, a mouse, a rat, a rodent, a non-human primate, a cat, a dog, a pig a cow, a goat, or a sheep. In some embodiments, the animal is a mouse. In some embodiments, the animal comprises or consists of transgenic cells that express an increased number of ube3a protein. In some embodiments, the animal comprises or consists of cells that comprise an increased copy number of a ube3a gene or of a ube3a-encoding nucleic acid sequence as compared to their wild-type counterpart. Typically, a wild-type cell comprises two copies of the ube3a gene, corresponding to two nucleic acid sequences encoding a ube3a protein. Accordingly, a genome comprising three copies of a nucleic acid encoding ube3a would be a genome comprising an increased copy number of a ube3a gene or of a ube3a-encoding nucleic acid sequence as compared to a wild-type genome. Similarly, a genome a genome comprising four copies of a nucleic acid encoding ube3a would be a genome comprising an increased copy number of a ube3a gene or of a ube3a-encoding nucleic acid sequence as compared to a wild-type genome. Genomes and cells comprising one extra copy of a ube3a-encoding nucleic acid sequence, for example, one extra copy of a ubde3a gene, are referred to herein as “1×” genomes or cells, while genomes comprising 2 extra copies are referred to as “2×” genomes or cells. Similarly, transgenic animals comprising cells having one extra copy of a ube3a-encoding nucleic acid in their genome are referred to as 1× transgenics, while animal comprising cells having two extra copies are referred to as 2× transgenics.

Some aspects of this invention provide genetically modified, or transgenic, cells comprising an extra copy of a ube3a-encoding nucleic acid sequence. In some embodiments, the cells do not comprise a dup15 or idic15 mutation. In some embodiments, the cells comprise a dup15 or idic15 mutation and at least one copy of an isolated ube3a-encoding nucleic acid sequence. In some embodiments, the extra copy of a ube3a-encoding sequence is stably integrated into the genome of the cell. In some embodiments, the cell comprises one isolated nucleic acid sequence encoding ube3a. In some embodiments, the cell comprises two isolated nucleic acid sequences encoding ube3a. In some embodiments, the cell comprises more than two isolated nucleic acid sequences encoding ube3a. In some embodiments, the cell comprises the extra copy or extra copies of ube3a-encoding nucleic acids in addition to any endogenous copies of the ube3a gene comprised in the genome of wild-type cells of the same genetic background.

Methods to genetically modify cells are well known to those of skill in the art and the invention is not limited in this respect. For example, additional copies of isolated nucleic acids can be introduced into the genome of a cell by electroporation of DNA constructs, for example, of expression constructs or of artificial chromosomes (e.g., bacterial artificial chromosomes (BACs)), by viral infection, or by transfection of DNA using a transfection agent such as LIPOFECTAMINE™ or FUGENE™. The term “stably integrated into a genome” refers to a nucleic acid sequence that is either integrated into a chromosome comprises in a cell or animal, e.g., into an endogenous chromosome or as part of an artificial chromosome, or is present in an extrachromosomal form that does not become diluted or lost during cell divisions during the life time of the cell. For example, a viral vector that does not integrate into the genome of a host cell, such as an adenoviral vector, is referred to as stably integrated into the genome of a cell, if the cell is a non-dividing cell, such as a post-mitotic neuron. In some embodiments, the cells are embryonic stem cells, for example, mouse or human embryonic stem cells. In some embodiments, the additional copy or copies of the nucleic acid encoding ube3a are targeted to a specific locus within the genome of the cell by homologous recombination. Gene targeting methods, reagents and strategies useful in such methods, as well as genetic loci suitable for genetic targeting are well known to those of skill in the art and the invention is not limited in this respect.

In some embodiments, the invention provides transgenic cells that express an increased amount of ube3a protein as compared to their wild type counterparts. In some embodiments, the cells express about 0.3, about 0.5, about 0.75, about 1, about 1.5, about 2, about 3, about 4, about 5, about 6, about 7, about 8, about 9, about 10, or more than about 10 times more ube3a protein as compared to their wild type counterparts, e.g., as measured in the amount of protein present in the cell, or as measured in the level of ube3a activity in the presence of a suitable substrate.

In some embodiments, the transgenic cells provided herein are non-human cells, for example, mouse cells. In some embodiments, the cells are derived from a mouse, for example, from an FVB mouse. In some embodiments, the cells are derived from a mouse having a normal genomic make-up. In some embodiments, the cells are derived from a mouse having a dup15 or idic15 mutation. Mice of other genetic backgrounds, or genomic make-ups are suitable for the derivation and generation of the transgenic cells described herein, as will be apparent to those of skill in the art, and the invention is not limited in this respect. In some embodiments, the cells are human cells. In some embodiments, the cells are embryonic stem cells. In some embodiments, the cells are neuronal cells.

In some embodiments, a cell provided herein is used in in vitro studies of the physiological and molecular pathologies associated with autism. For example, in some embodiments, an extra copy or extra copies of a nucleic acid encoding a ube3a protein are introduced into the genome of an embryonic stem cell, for example, a mouse or a human embryonic stem cell, and the cell or its progeny is differentiated into a neuronal cell, for example, by methods of cell differentiation well known to those of skill in the art. In some embodiments, the neuronal cell is then used in an in vitro assay, for example, in an assay measuring a characteristic of a neuronal cell, such as number and/or structure of synaptic connections, electrophysiological cell properties, or expression analysis (e.g., immunocytochemistry). In some embodiments, the differentiated cell is used in a drug screening assay, for example, in a screening assay to identify a drug that effects a change of a parameter that is altered in the ube3a-transgenic neuronal cells provided herein as compared to a wild type cell of the same neuronal cell type, in a manner that changes the altered parameter towards the state of the parameter in the wild type cell. For example, in some embodiments, a neuronal 1× cell or a 2× cell as provided herein (comprising 1 or 2 extra copies of a ube3a-encoding nucleic acid expression construct, respectively) is used to screen for a drug that alleviates the impairment of presynaptic glutamate release that is typically observed in these cells as described elsewhere herein in more detail.

In some embodiments, an additional copy of a nucleic acid encoding ube3a is introduced into a cell as part of an expression construct. An expression construct typically comprises a coding sequence, for example, a nucleic acid sequence encoding ube3a, and a promoter driving transcription of the coding sequence. In some embodiments, the coding sequence is a ube3a cDNA. In some embodiments, the coding sequence is a ube3a gene sequence, for example, the entire intron-exon sequence of a ube3a gene. In some embodiments, the expression construct comprises an isolated ube3a gene, or at least the region of the gene comprising the ube3a coding sequence and the ube3a promoter. The transgenic mammalian cell of any of claims 1-11, wherein the one or more isolated nucleic acid sequence(s) encoding a ube3a protein comprise a ube3a cDNA. In some embodiments, a cell is provided that comprises one or more isolated nucleic acid sequence(s) encoding a ube3a protein, comprise a ube3a-encoding genomic region. In some embodiments, the one or more isolated nucleic acid sequence(s) encoding a ube3a protein comprise an isolated genomic fragment comprising a wild-type ube3a coding sequence, for example, an isolated ube3a gene. In some embodiments, the one or more isolated nucleic acid sequence(s) encoding a ube3a protein comprise a synthetic ube3a coding sequence, for example, a sequence optimized for codon usage in the cell. In some embodiments, the ube3a coding sequence is a mouse or a human ube3a coding sequence. Mouse and human ube3a coding sequences and protein sequences are well known to those of skill in the art. Some representative ube3a transcript sequences are listed below, and additional ube3a encoding sequences, including further transcript sequences, but also genomic encoding sequences and recombinant and synthetic sequences will be apparent to those of skill in the art. The invention is not limited to transcript or cDNA sequences.

>gi|146198826|ref|NM_173010.3| Mus musculus ubiquitin protein ligase E3A (Ube3a), transcript
variant 1, mRNA (SEQ ID NO: 1)
CTGTCGGGATACTCGGTCCGCCCACCTAGTCCTCTCGTCCAGTGCTGCGTTCGCGAGATCCGTATTTCTCCCAAGATGGTGGCGCTCCTCTTTG
GGTGACTCCAGGAGACGACAGGGCCTTTCGTCTTTGCCAGCACCTCGTCGCCCCTCCTGCGCTCGCTCTCTCGCTCGCGCACCGGGCCACGCAG
CTGTTCACCGCCTCGTTACGCTTCTCTTCCGTCGACCTGTCGCTGACGGTGGCGCCTCCTTCTGCTTCTCTTCGGAGTTGCTCGCCGCCCTCGC
CCCCCACTGTGGACAGATCGCGACAGCAGCGCTTCAGCGCCGACTTCAAGGTTGCCCAGGCGCCTGGCCTCTCGGCCTCGGTTTCCTGAGGAGA
AGCGCGGGTCCCGCATGAGACCCGGCGGTGGCGCCAGCGAAAGGGAACGAGGCGGTGGCGGGCGGCGGCGGTGGACGAGGGCGACAAGGACCAG
TGAGGCGGCCGCAGCTGCGAGGGCCGCAGCCCACGCGCGGGGGCGAGGACAGATCACCAGGAGAATCCCAGTCTGAGGACATTGAAGCTAGCCG
AATGAAGCGAGCAGCTGCAAAGCATCTAATAGAACGCTACTACCATCAGTTAACTGAGGGCTGTGGAAATGAGGCCTGCACGAATGAGTTTTGT
GCTTCCTGTCCAACTTTTCTTCGTATGGATAACAATGCAGCAGCTATTAAAGCCCTTGAGCTTTATAAAATTAATGCAAAACTCTGTGATCCTC
ATCCCTCCAAGAAAGGAGCAAGCTCAGCTTACCTTGAGAACTCAAAAGGTGCATCTAACAACTCAGAGATAAAAATGAACAAGAAGGAAGGAAA
AGATTTTAAAGATGTGATTTACCTAACTGAAGAGAAAGTATATGAAATTTATGAATTTTGTAGAGAGAGTGAGGATTATTCCCCTTTAATTCGT
GTAATTGGAAGAATATTTTCTAGTGCTGAGGCACTGGTTCTGAGCTTTCGGAAAGTCAAACAGCACACAAAGGAGGAATTGAAATCTCTTCAAG
AAAAGGATGAAGACAAGGATGAAGATGAAAAGGAAAAAGCTGCATGTTCTGCTGCTGCTATGGAAGAAGACTCAGAAGCATCTTCTTCAAGGAT
GGGTGATAGTTCACAGGGAGACAACAATGTACAAAAATTAGGTCCTGATGATGTGACTGTGGATATTGATGCTATTAGAAGGGTCTACAGCAGT
TTGCTCGCTAATGAAAAATTAGAAACTGCCTTCCTGAATGCACTTGTATATCTGTCACCTAACGTGGAATGTGATTTGACATATCATAATGTGT
ATACTCGAGATCCTAATTATCTCAATTTGTTCATTATTGTAATGGAGAATAGTAATCTCCACAGTCCTGAATATCTGGAAATGGCGTTGCCATT
ATTTTGCAAAGCTATGTGTAAGCTACCCCTTGAAGCTCAAGGAAAACTGATTAGGCTGTGGTCTAAATACAGTGCTGACCAGATTCGGAGAATG
ATGGAAACATTTCAGCAACTTATTACCTACAAAGTCATAAGCAATGAATTTAATAGCCGAAATCTAGTGAATGATGATGATGCCATTGTTGCTG
CTTCAAAGTGTTTGAAAATGGTTTACTATGCAAATGTAGTGGGAGGGGATGTGGACACAAATCATAATGAGGAAGATGATGAAGAACCCATACC
TGAGTCCAGCGAATTAACACTTCAGGAGCTTCTGGGAGATGAAAGAAGAAATAAGAAAGGTCCTCGAGTGGATCCACTAGAAACCGAACTTGGC
GTTAAAACTCTAGACTGTCGAAAACCACTTATCTCCTTTGAAGAATTCATTAATGAACCACTGAATGATGTTCTAGAAATGGACAAAGATTATA
CCTTTTTCAAAGTTGAAACAGAGAACAAATTCTCTTTTATGACATGTCCCTTTATATTGAATGCTGTCACAAAGAATCTGGGATTATATTATGA
CAATAGAATTCGCATGTACAGTGAAAGAAGAATCACTGTTCTTTACAGCCTAGTTCAAGGACAGCAGTTGAATCCGTATTTGAGACTCAAAGTC
AGACGTGACCATATTATAGATGATGCACTGGTCCGGCTAGAGATGATTGCTATGGAAAATCCTGCAGACTTGAAGAAGCAGTTGTATGTGGAAT
TTGAAGGAGAACAAGGAGTAGATGAGGGAGGCGTTTCCAAAGAGTTTTTTCAGTTGGTTGTGGAGGAAATTTTTAATCCAGATATTGGTATGTT
CACATATGATGAAGCTACGAAATTATTTTGGTTTAATCCATCTTCTTTTGAAACTGAGGGTCAGTTTACTCTGATTGGCATAGTCCTGGGTCTG
GCTATTTACAATAATTGTATACTGGATGTCCATTTTCCCATGGTTGTATACAGGAAGCTAATGGGGAAAAAAGGAACCTTTCGTGACTTGGGAG
ACTCTCACCCAGTTTTATATCAGAGTTTAAAGGATTTATTGGAATATGAAGGGAGTGTGGAAGATGATATGATGATCACTTTCCAGATATCACA
GACAGATCTTTTTGGTAACCCAATGATGTATGATCTAAAAGAAAATGGTGATAAAATTCCAATTACAAATGAAAACAGGAAGGAATTTGTCAAT
CTCTATTCAGACTACATTCTCAATAAATCTGTAGAAAAACAATTCAAGGCATTTCGCAGAGGTTTTCATATGGTGACTAATGAATCGCCCTTAA
AATACTTATTCAGACCAGAAGAAATTGAATTGCTTATATGTGGAAGCCGGAATCTAGATTTCCAGGCACTAGAAGAAACTACAGAGTATGACGG
TGGCTATACGAGGGAATCTGTTGTGATTAGGTAAGGTGTTTAATTCTTAAAAAGGAAGATTTTATTCATCAAACATGTAGATGTGTGCTTTTGT
GTCCTGTATCTGTAGGTACTGGTTACCAAACAAGTAAGCTCAAAAATAGACCTGTATTAATATTTCCAATTTTCATGCAGTCTAATGCTTTATT
TCATGAATTAAATGATTTAAGTCTCATATTTTCTCAACCCTTTGCCTTATTTTTGGTCATGTGTAAGATGGCACATTATTTAGTCTTTAAGATA
CTTGGGAAGAACCATGTATACTAGTGATTCTGAACAATTCTTAGGACAGTATTACCACTAACATCGTTCTCTAGTCAAATGCCCTTATTTCTAC
TTCTGTAATATGCTACTATCCAATTCTGAAAGATCTTTCCCCCCATCTTCTAATGTGACTGATCAAAATGCAGAGTAGTCTTTTTGGCATCCAC
TATGATGTCATAGGTATTTAAACAGTTATCTTTTTGTAGATCACTTGAGCTATAAGACTCAAATATGTTAACAATAGAATGAATATTAACTGTG
TCTAGTAATGATACATTATCATTGTTATATTTATATTACAGTATTACTTTATTCATTTAAGTTTGTAGAAGATTACTCTTGCTTTGCCCTTTTT
TTTTTAATAGAAAAGCAAATATGTTATTTATTCAGCTTTTAGGTAATTAAATAACAAAATTCAGAGTAAAGCAAAACAAAAACCATAACATGTC
ATATGATATATCATTTCTAAGCACAATGGCAATTATTAATGAATATAAAAATTTATCATTCATATTTGCTTCTAACACCAGTCACAAAAGTGGC
AACCATTATATTGCTGCTCAGTTTTAAAGGTAATTCATAACAGGGATAAACATGGTAATACAGAAGCCTTAATGGGAATATCCTAGTATTATCT
CTACAATATGGCAAAATAATGTTTTAGATTGATTATGATTAATGTATGCATTTTGATTATTATCCTTTTGTTATTGGCAATAGAATTATCATGA
CAGTGGGGCTGTTACAAATAAAGTTTTCATTCTT
>gi|76880499|ref|NM_001033962.1| Mus musculus ubiquitin protein ligase E3A (Ube3a), transcript
variant 3, mRNA (SEQ ID NO: 2)
GTCGGGATACTCGGTCCGCCCACCTAGTCCTCTCGTCCAGTGCTGCGTTCGCGAGATCCGTATTTCTCCCAAGATGGTGGCGCTCCTCTTTGGG
TGACTCCAGGAGACGACAGGGCCTTTCGTCTTTGCCAGCACCTCGTCGCCCCTCCTGCGCTCGCTCTCTCGCTCGCGCACCGGGCCACGCAGCT
GTTCACCGCCTCGTTACGCTTCTCTTCCGTCGACCTGTCGCTGACGGTGGCGCCTCCTTCTGCTTCTCTTCGGAGTTGCTCGCCGCCCTCGCCC
CCCACTGTGGACAGATCGCGACAGCAGCGCTTCAGCGCCGACTTCAAGGTTGCCCAGGCGCCTGGCCTCTCGGCCTCGGTTTCCTGAGGAGAAG
CGCGGGTCCCGCATGAGACCCGGCGGTGGCGCCAGCGAAAGGGAACGAGGCGGTGGCGGGCGGCGGCGGTGGACGAGGGCGACAAGGACCAGTG
AGGCGGCCGCAGCTGCGAGGGCCGCAGCCCACGCGCGGGGGCGAGGACAGATCACCAGGAGAATCCCAGTCTGAGGACATTGAAGCTAGCCGAA
TGAAGCGAGCAGCTGCAAAGCATCTAATAGAACGCTACTACCATCAGTTAACTGAGGGCTGTGGAAATGAGGCCTGCACGAATGAGTTTTGTGC
TTCCTGTCCAACTTTTCTTCGTATGGATAACAATGCAGCAGCTATTAAAGCCCTTGAGCTTTATAAAATTAATGCAAAACTCTGTGATCCTCAT
CCCTCCAAGAAAGGAGCAAGCTCAGCTTACCTTGAGAACTCAAAAGGTGCATCTAACAACTCAGAGATAAAAATGAACAAGAAGGAAGGAAAAG
ATTTTAAAGATGTGATTTACCTAACTGAAGAGAAAGTATATGAAATTTATGAATTTTGTAGAGAGAGTGAGGATTATTCCCCTTTAATTCGTGT
AATTGGAAGAATATTTTCTAGTGCTGAGGCACTGGTTCTGAGCTTTCGGAAAGTCAAACAGCACACAAAGGAGGAATTGAAATCTCTTCAAGAA
AAGGATGAAGACAAGGATGAAGATGAAAAGGAAAAAGCTGCATGTTCTGCTGCTGCTATGGAAGAAGACTCAGAAGCATCTTCTTCAAGGATGG
GTGATAGTTCACAGGGAGACAACAATGTACAAAAATTAGGTCCTGATGATGTGACTGTGGATATTGATGCTATTAGAAGGGTCTACAGCAGTTT
GCTCGCTAATGAAAAATTAGAAACTGCCTTCCTGAATGCACTTGTATATCTGTCACCTAACGTGGAATGTGATTTGACATATCATAATGTGTAT
ACTCGAGATCCTAATTATCTCAATTTGTTCATTATTGTAATGGAGAATAGTAATCTCCACAGTCCTGAATATCTGGAAATGGCGTTGCCATTAT
TTTGCAAAGCTATGTGTAAGCTACCCCTTGAAGCTCAAGGAAAACTGATTAGGCTGTGGTCTAAATACAGTGCTGACCAGATTCGGAGAATGAT
GGAAACATTTCAGCAACTTATTACCTACAAAGTCATAAGCAATGAATTTAATAGCCGAAATCTAGTGAATGATGATGATGCCATTGTTGCTGCT
TCAAAGTGTTTGAAAATGGTTTACTATGCAAATGTAGTGGGAGGGGATGTGGACACAAATCATAATGAGGAAGATGATGAAGAACCCATACCTG
AGTCCAGCGAATTAACACTTCAGGAGCTTCTGGGAGATGAAAGAAGAAATAAGAAAGGTCCTCGAGTGGATCCACTAGAAACCGAACTTGGCGT
TAAAACTCTAGACTGTCGAAAACCACTTATCTCCTTTGAAGAATTCATTAATGAACCACTGAATGATGTTCTAGAAATGGACAAAGATTATACC
TTTTTCAAAGTTGAAACAGAGAACAAATTCTCTTTTATGACATGTCCCTTTATATTGAATGCTGTCACAAAGAATCTGGGATTATATTATGACA
ATAGAATTCGCATGTACAGTGAAAGAAGAATCACTGTTCTTTACAGCCTAGTTCAAGGACAGCAGTTGAATCCGTATTTGAGACTCAAAGTCAG
ACGTGACCATATTATAGATGATGCACTGGTCCGGCTAGAGATGATTGCTATGGAAAATCCTGCAGACTTGAAGAAGCAGTTGTATGTGGAATTT
GAAGGAGAACAAGGAGTAGATGAGGGAGGCGTTTCCAAAGAGTTTTTTCAGTTGGTTGTGGAGGAAATTTTTAATCCAGATATTGGTATGTTCA
CATATGATGAAGCTACGAAATTATTTTGGTTTAATCCATCTTCTTTTGAAACTGAGGGTCAGTTTACTCTGATTGGCATAGTCCTGGGTCTGGC
TATTTACAATAATTGTATACTGGATGTCCATTTTCCCATGGTTGTATACAGGAAGCTAATGGGGAAAAAAGGAACCTTTCGTGACTTGGGAGAC
TCTCACCCAGTTTTATATCAGAGTTTAAAGGATTTATTGGAATATGAAGGGAGTGTGGAAGATGATATGATGATCACTTTCCAGATATCACAGA
CAGATCTTTTTGGTAACCCAATGATGTATGATCTAAAAGAAAATGGTGATAAAATTCCAATTACAAATGAAAACAGGAAGGAATTTGTCAATCT
CTATTCAGACTACATTCTCAATAAATCTGTAGAAAAACAATTCAAGGCATTTCGCAGAGGTTTTCATATGGTGACTAATGAATCGCCCTTAAAA
TACTTATTCAGACCAGAAGAAATTGAATTGCTTATATGTGGAAGCCGGAATCTAGATTTCCAGGCACTAGAAGAAACTACAGAGTATGACGGTG
GCTATACGAGGGAATCTGTTGTGATTAGGGAGTTCTGGGAAATTGTTCATTCGTTTACAGATGAACAGAAAAGACTCTTTCTGCAGTTTACAAC
AGGCACAGACAGAGCACCTGTTGGAGGACTAGGAAAATTGAAGATGATTATAGCCAAAAATGGCCCAGACACAGAAAGGTTACCTACATCTCAT
ACTTGCTTTAATGTCCTTTTACTTCCGGAATATTCAAGCAAAGAAAAACTTAAAGAGAGATTGTTGAAGGCCATCACATATGCCAAAGGATTTG
GCATGCTGTAAACAAAAAGAAAAAGAAAAAGAAAAAGAAAAAGTTAAAAAATAAATATAAGAGGGATAATTTGATGGTAATAGTATCCCAGTAC
AAAAAGGCTGTAAGATAGTGAACCACAGTAGTCATCTATGTCTGTGCCTCCCTTCTTCATTGGGGACATTGTGGGCTGGAACAGCAGATTTCAG
CTGCATATATGAACAAATCCTTTATTATTATTATAATTATTTTTTTGCGTGAAAGTGTTACATATTCTTTCACTTGTATGTACAGAGAGGTTTT
CTGAATATTTATTTTAAGGGTTAAATCACTTTTGCTTGTGTTTATTACTGCTTGAGGTTGAGCCTTTTTGAGTATTTAAGATATATATACCAAC
GAAACTATTCTCGCAAGGAAAACATTGCCACCATTTGTAGAACATGTAATCTTCAAGTATGTGCTATTTTTTGTCCCTGTATCTAAGTCAAATC
AGGAACTTTTTTCTAACAATTTGCTTTTGAAACTTGAAGTCAAGGAAACAGTGTGGTGCAAGTACTGCTGTTCTAGCCCCCAAAGAGTTTTCTG
TACAAAATTTTGAGAACCAATAAAGATGGAAGGGAGAACTTGGAATGTTTGAACCACAGCCCTCAGAACTTTAGTAACAGCACAACAAATTAAA
ACAACTCATGCCACAGTATGTTGTCTTCATGTGTCTTGCAATGAACTGTTTCAGTAGCCAATCCTCTTAGTATATGAAAGGACAGGGATTTTTT
TTTTGTTTTTGTTGTTGTTGTTGTTGTTGTTGTTTTTGTTGTTGTTGTTGTTTTTGTTGTTTAAGTTTACTGGGGAAAGTGCATCTGGCCAAAT
GATAGGATAGTCAAGCCTATTGCAACAAAATTAGGAAGTTTGTTGTATAAATAAGCATGTAAAAGTGCACTTAAAATGAATCTTTATTATTGCT
GAGATTTTAATAGACAATCCAAAGTCTCCCCTTCTGTTGCCGTCATCTTGTTTAATCAACCATTTTTCAAGGCACTCGATCAGTGTTGCAGCAT
AACAGAAAGTACAGCTACTGTGCCTTGTGTTACTTATTTACACAGTTAGCAGGCCTGGAAATGAATGGAACTAGTACTCCTGAGAAATAAATTG
TATATCCCCCAAATTAAAATTTACTTCAAAGGTGTTAAAGATTTCATGTCCTATATTAAAGTACAAATAGGCTTAAATTACTGGATATTTAATG
TAGTTTCCCATCCCTAGTCTTCTATGTCTGTGATGTTAATTTCTTTTGTTGCATAACAAAATAAAAGAATTATGTATTTTTAACTAAGGAGAGA
CATACTGGTATATCATTTTACTACAAGCTACAGATAACCTGTTGAGCTTGTGCCTTGATTGTTTTAACAACTAGTGCAAATCAACCTGATGATT
TTAATTGGCAGGGGATAATGGTAGCTTTCAAATCATTGGAAGGGGAAAAGGATGTCTTAGGATTATTTTCTTTCTTGTAGTAGTTGAGACAGAG
CTCTTATTTACTGTAATGCTAAATGAAACAGTGGCTTAAATATTTTAATGGGAAAAGAGAACACAGTGCGTTCCATATTGTGATAAGGTAACGT
GAGGTTTTTTTGTTTTGTTTTGTTTTCTTTTTTTTTTTTCTGAGCTAGCCTTTAGAACACTGTTGTGGTATGTATGCTACCTTGATTATAGGAC
CCCCTAAATGTGACTATAGTCATCTTAATGGGCATCTTGTCCACTGTGCTTCTTATGTATTATGAAAGTGATAAGAAGACAAATTAAGTGGGTA
TATTTTATAAAATAAATTCATG
>gi|76880493|ref|NM_011668.2| Mus musculus ubiquitin protein ligase E3A (Ube3a), transcript
variant 2, mRNA (SEQ ID NO: 3)
GTCGGGATACTCGGTCCGCCCACCTAGTCCTCTCGTCCAGTGCTGCGTTCGCGAGATCCGTATTTCTCCCAAGATGGTGGCGCTCCTCTTTGGG
TGACTCCAGGAGACGACAGGGCCTTTCGTCTTTGCCAGCACCTCGTCGCCCCTCCTGCGCTCGCTCTCTCGCTCGCGCACCGGGCCACGCAGCT
GTTCACCGCCTCGTTACGCTTCTCTTCCGTCGACCTGTCGCTGACGGTGGCGCCTCCTTCTGCTTCTCTTCGGAGTTGCTCGCCGCCCTCGCCC
CCCACTGTGGACAGATCGCGACAGCAGCGCTTCAGCGCCGACTTCAAGGTTGCCCAGGCGCCTGGCCTCTCGGCCTCGGTTTCCTGAGGAGAAG
CGCGGGTCCCGCATGAGACCCGGCGGTGGCGCCAGCGAAAGGGAACGAGGCGGTGGCGGGCGGCGGCGGTGGACGAGGGCGACAAGGACCAGTG
AGGCGGCCGCAGCTGCGAGGGCCGCAGCCCACGCGCGGGGGCGAGGACAGGTTAAAAAATCTCTCTAAGAGCCTGATTTTAGAGTTCACCAGCT
CCTCAGAAGTTTGGCGAAATATGAATTATTAAGCCTACGTTCAGATCAAGTTAGCAGCTAGACTGGTGTGACAACCTGTTTTTAATCAGTGACT
CAAAGCTGTTATCACCCTGATGTCACCGAATGGCCACAGCTTGTAAAAGATCACCAGGAGAATCCCAGTCTGAGGACATTGAAGCTAGCCGAAT
GAAGCGAGCAGCTGCAAAGCATCTAATAGAACGCTACTACCATCAGTTAACTGAGGGCTGTGGAAATGAGGCCTGCACGAATGAGTTTTGTGCT
TCCTGTCCAACTTTTCTTCGTATGGATAACAATGCAGCAGCTATTAAAGCCCTTGAGCTTTATAAAATTAATGCAAAACTCTGTGATCCTCATC
CCTCCAAGAAAGGAGCAAGCTCAGCTTACCTTGAGAACTCAAAAGGTGCATCTAACAACTCAGAGATAAAAATGAACAAGAAGGAAGGAAAAGA
TTTTAAAGATGTGATTTACCTAACTGAAGAGAAAGTATATGAAATTTATGAATTTTGTAGAGAGAGTGAGGATTATTCCCCTTTAATTCGTGTA
ATTGGAAGAATATTTTCTAGTGCTGAGGCACTGGTTCTGAGCTTTCGGAAAGTCAAACAGCACACAAAGGAGGAATTGAAATCTCTTCAAGAAA
AGGATGAAGACAAGGATGAAGATGAAAAGGAAAAAGCTGCATGTTCTGCTGCTGCTATGGAAGAAGACTCAGAAGCATCTTCTTCAAGGATGGG
TGATAGTTCACAGGGAGACAACAATGTACAAAAATTAGGTCCTGATGATGTGACTGTGGATATTGATGCTATTAGAAGGGTCTACAGCAGTTTG
CTCGCTAATGAAAAATTAGAAACTGCCTTCCTGAATGCACTTGTATATCTGTCACCTAACGTGGAATGTGATTTGACATATCATAATGTGTATA
CTCGAGATCCTAATTATCTCAATTTGTTCATTATTGTAATGGAGAATAGTAATCTCCACAGTCCTGAATATCTGGAAATGGCGTTGCCATTATT
TTGCAAAGCTATGTGTAAGCTACCCCTTGAAGCTCAAGGAAAACTGATTAGGCTGTGGTCTAAATACAGTGCTGACCAGATTCGGAGAATGATG
GAAACATTTCAGCAACTTATTACCTACAAAGTCATAAGCAATGAATTTAATAGCCGAAATCTAGTGAATGATGATGATGCCATTGTTGCTGCTT
CAAAGTGTTTGAAAATGGTTTACTATGCAAATGTAGTGGGAGGGGATGTGGACACAAATCATAATGAGGAAGATGATGAAGAACCCATACCTGA
GTCCAGCGAATTAACACTTCAGGAGCTTCTGGGAGATGAAAGAAGAAATAAGAAAGGTCCTCGAGTGGATCCACTAGAAACCGAACTTGGCGTT
AAAACTCTAGACTGTCGAAAACCACTTATCTCCTTTGAAGAATTCATTAATGAACCACTGAATGATGTTCTAGAAATGGACAAAGATTATACCT
TTTTCAAAGTTGAAACAGAGAACAAATTCTCTTTTATGACATGTCCCTTTATATTGAATGCTGTCACAAAGAATCTGGGATTATATTATGACAA
TAGAATTCGCATGTACAGTGAAAGAAGAATCACTGTTCTTTACAGCCTAGTTCAAGGACAGCAGTTGAATCCGTATTTGAGACTCAAAGTCAGA
CGTGACCATATTATAGATGATGCACTGGTCCGGCTAGAGATGATTGCTATGGAAAATCCTGCAGACTTGAAGAAGCAGTTGTATGTGGAATTTG
AAGGAGAACAAGGAGTAGATGAGGGAGGCGTTTCCAAAGAGTTTTTTCAGTTGGTTGTGGAGGAAATTTTTAATCCAGATATTGGTATGTTCAC
ATATGATGAAGCTACGAAATTATTTTGGTTTAATCCATCTTCTTTTGAAACTGAGGGTCAGTTTACTCTGATTGGCATAGTCCTGGGTCTGGCT
ATTTACAATAATTGTATACTGGATGTCCATTTTCCCATGGTTGTATACAGGAAGCTAATGGGGAAAAAAGGAACCTTTCGTGACTTGGGAGACT
CTCACCCAGTTTTATATCAGAGTTTAAAGGATTTATTGGAATATGAAGGGAGTGTGGAAGATGATATGATGATCACTTTCCAGATATCACAGAC
AGATCTTTTTGGTAACCCAATGATGTATGATCTAAAAGAAAATGGTGATAAAATTCCAATTACAAATGAAAACAGGAAGGAATTTGTCAATCTC
TATTCAGACTACATTCTCAATAAATCTGTAGAAAAACAATTCAAGGCATTTCGCAGAGGTTTTCATATGGTGACTAATGAATCGCCCTTAAAAT
ACTTATTCAGACCAGAAGAAATTGAATTGCTTATATGTGGAAGCCGGAATCTAGATTTCCAGGCACTAGAAGAAACTACAGAGTATGACGGTGG
CTATACGAGGGAATCTGTTGTGATTAGGGAGTTCTGGGAAATTGTTCATTCGTTTACAGATGAACAGAAAAGACTCTTTCTGCAGTTTACAACA
GGCACAGACAGAGCACCTGTTGGAGGACTAGGAAAATTGAAGATGATTATAGCCAAAAATGGCCCAGACACAGAAAGGTTACCTACATCTCATA
CTTGCTTTAATGTCCTTTTACTTCCGGAATATTCAAGCAAAGAAAAACTTAAAGAGAGATTGTTGAAGGCCATCACATATGCCAAAGGATTTGG
CATGCTGTAAACAAAAAGAAAAAGAAAAAGAAAAAGAAAAAGTTAAAAAATAAATATAAGAGGGATAATTTGATGGTAATAGTATCCCAGTACA
AAAAGGCTGTAAGATAGTGAACCACAGTAGTCATCTATGTCTGTGCCTCCCTTCTTCATTGGGGACATTGTGGGCTGGAACAGCAGATTTCAGC
TGCATATATGAACAAATCCTTTATTATTATTATAATTATTTTTTTGCGTGAAAGTGTTACATATTCTTTCACTTGTATGTACAGAGAGGTTTTC
TGAATATTTATTTTAAGGGTTAAATCACTTTTGCTTGTGTTTATTACTGCTTGAGGTTGAGCCTTTTTGAGTATTTAAGATATATATACCAACG
AAACTATTCTCGCAAGGAAAACATTGCCACCATTTGTAGAACATGTAATCTTCAAGTATGTGCTATTTTTTGTCCCTGTATCTAAGTCAAATCA
GGAACTTTTTTCTAACAATTTGCTTTTGAAACTTGAAGTCAAGGAAACAGTGTGGTGCAAGTACTGCTGTTCTAGCCCCCAAAGAGTTTTCTGT
ACAAAATTTTGAGAACCAATAAAGATGGAAGGGAGAACTTGGAATGTTTGAACCACAGCCCTCAGAACTTTAGTAACAGCACAACAAATTAAAA
CAACTCATGCCACAGTATGTTGTCTTCATGTGTCTTGCAATGAACTGTTTCAGTAGCCAATCCTCTTAGTATATGAAAGGACAGGGATTTTTTT
TTTGTTTTTGTTGTTGTTGTTGTTGTTGTTGTTTTTGTTGTTGTTGTTGTTTTTGTTGTTTAAGTTTACTGGGGAAAGTGCATCTGGCCAAATG
ATAGGATAGTCAAGCCTATTGCAACAAAATTAGGAAGTTTGTTGTATAAATAAGCATGTAAAAGTGCACTTAAAATGAATCTTTATTATTGCTG
AGATTTTAATAGACAATCCAAAGTCTCCCCTTCTGTTGCCGTCATCTTGTTTAATCAACCATTTTTCAAGGCACTCGATCAGTGTTGCAGCATA
ACAGAAAGTACAGCTACTGTGCCTTGTGTTACTTATTTACACAGTTAGCAGGCCTGGAAATGAATGGAACTAGTACTCCTGAGAAATAAATTGT
ATATCCCCCAAATTAAAATTTACTTCAAAGGTGTTAAAGATTTCATGTCCTATATTAAAGTACAAATAGGCTTAAATTACTGGATATTTAATGT
AGTTTCCCATCCCTAGTCTTCTATGTCTGTGATGTTAATTTCTTTTGTTGCATAACAAAATAAAAGAATTATGTATTTTTAACTAAGGAGAGAC
ATACTGGTATATCATTTTACTACAAGCTACAGATAACCTGTTGAGCTTGTGCCTTGATTGTTTTAACAACTAGTGCAAATCAACCTGATGATTT
TAATTGGCAGGGGATAATGGTAGCTTTCAAATCATTGGAAGGGGAAAAGGATGTCTTAGGATTATTTTCTTTCTTGTAGTAGTTGAGACAGAGC
TCTTATTTACTGTAATGCTAAATGAAACAGTGGCTTAAATATTTTAATGGGAAAAGAGAACACAGTGCGTTCCATATTGTGATAAGGTAACGTG
AGGTTTTTTTGTTTTGTTTTGTTTTCTTTTTTTTTTTTCTGAGCTAGCCTTTAGAACACTGTTGTGGTATGTATGCTACCTTGATTATAGGACC
CCCTAAATGTGACTATAGTCATCTTAATGGGCATCTTGTCCACTGTGCTTCTTATGTATTATGAAAGTGATAAGAAGACAAATTAAGTGGGTAT
ATTTTATAAAATAAATTCATG
>gi|219801927|ref|NG_009268.1| Homo sapiens ubiquitin protein ligase E3A (UBE3A), RefSeqGene
(LRG_15) on chromosome 15 (NCBI database entry (www.ncbi.nlm.nih.gov, incorporated herein in
its entirety by reference).
>gi|332000023|ref|NM_000462.3| Homo sapiens ubiquitin protein ligase E3A (UBE3A), transcript
variant 2, mRNA (SEQ ID NO: 4)
AGCCAGTCCTCCCGTCTTGCGCCGCGGCCGCGAGATCCGTGTGTCTCCCAAGATGGTGGCGCTGGGCTCGGGGTGACTACAGGAGACGACGGGG
CCTTTTCCCTTCGCCAGGACCCGACACACCAGGCTTCGCTCGCTCGCGCACCCCTCCGCCGCGTAGCCATCCGCCAGCGCGGGCGCCCGCCATC
CGCCGCCTACTTACGCTTCACCTCTGCCGACCCGGCGCGCTCGGCTGCGGGCGGCGGCGCCTCCTTCGGCTCCTCCTCGGAATAGCTCGCGGCC
TGTAGCCCCTGGCAGGAGGGCCCCTCAGCCCCCCGGTGTGGACAGGCAGCGGCGGCTGGCGACGAACGCCGGGATTTCGGCGGCCCCGGCGCTC
CCTTTCCCGGCCTCGTTTTCCGGATAAGGAAGCGCGGGTCCCGCATGAGCCCCGGCGGTGGCGGCAGCGAAAGAGAACGAGGCGGTGGCGGGCG
GAGGCGGCGGGCGAGGGCGACTACGACCAGTGAGGCGGCCGCCGCAGCCCAGGCGCGGGGGCGACGACAGGTTAAAAATCTGTAAGAGCCTGAT
TTTAGAATTCACCAGCTCCTCAGAAGTTTGGCGAAATATGAGTTATTAAGCCTACGCTCAGATCAAGGTAGCAGCTAGACTGGTGTGACAACCT
GTTTTTAATCAGTGACTCAAAGCTGTGATCACCCTGATGTCACCGAATGGCCACAGCTTGTAAAAGAGAGTTACAGTGGAGGTAAAAGGAGTGG
CTTGCAGGATGGAGAAGCTGCACCAGTGTTATTGGAAATCAGGAGAACCTCAGTCTGACGACATTGAAGCTAGCCGAATGAAGCGAGCAGCTGC
AAAGCATCTAATAGAACGCTACTACCACCAGTTAACTGAGGGCTGTGGAAATGAAGCCTGCACGAATGAGTTTTGTGCTTCCTGTCCAACTTTT
CTTCGTATGGATAATAATGCAGCAGCTATTAAAGCCCTCGAGCTTTATAAGATTAATGCAAAACTCTGTGATCCTCATCCCTCCAAGAAAGGAG
CAAGCTCAGCTTACCTTGAGAACTCGAAAGGTGCCCCCAACAACTCCTGCTCTGAGATAAAAATGAACAAGAAAGGCGCTAGAATTGATTTTAA
AGATGTGACTTACTTAACAGAAGAGAAGGTATATGAAATTCTTGAATTATGTAGAGAAAGAGAGGATTATTCCCCTTTAATCCGTGTTATTGGA
AGAGTTTTTTCTAGTGCTGAGGCATTGGTACAGAGCTTCCGGAAAGTTAAACAACACACCAAGGAAGAACTGAAATCTCTTCAAGCAAAAGATG
AAGACAAAGATGAAGATGAAAAGGAAAAAGCTGCATGTTCTGCTGCTGCTATGGAAGAAGACTCAGAAGCATCTTCCTCAAGGATAGGTGATAG
CTCACAGGGAGACAACAATTTGCAAAAATTAGGCCCTGATGATGTGTCTGTGGATATTGATGCCATTAGAAGGGTCTACACCAGATTGCTCTCT
AATGAAAAAATTGAAACTGCCTTTCTCAATGCACTTGTATATTTGTCACCTAACGTGGAATGTGACTTGACGTATCACAATGTATACTCTCGAG
ATCCTAATTATCTGAATTTGTTCATTATCGTAATGGAGAATAGAAATCTCCACAGTCCTGAATATCTGGAAATGGCTTTGCCATTATTTTGCAA
AGCGATGAGCAAGCTACCCCTTGCAGCCCAAGGAAAACTGATCAGACTGTGGTCTAAATACAATGCAGACCAGATTCGGAGAATGATGGAGACA
TTTCAGCAACTTATTACTTATAAAGTCATAAGCAATGAATTTAACAGTCGAAATCTAGTGAATGATGATGATGCCATTGTTGCTGCTTCGAAGT
GCTTGAAAATGGTTTACTATGCAAATGTAGTGGGAGGGGAAGTGGACACAAATCACAATGAAGAAGATGATGAAGAGCCCATCCCTGAGTCCAG
CGAGCTGACACTTCAGGAACTTTTGGGAGAAGAAAGAAGAAACAAGAAAGGTCCTCGAGTGGACCCCCTGGAAACTGAACTTGGTGTTAAAACC
CTGGATTGTCGAAAACCACTTATCCCTTTTGAAGAGTTTATTAATGAACCACTGAATGAGGTTCTAGAAATGGATAAAGATTATACTTTTTTCA
AAGTAGAAACAGAGAACAAATTCTCTTTTATGACATGTCCCTTTATATTGAATGCTGTCACAAAGAATTTGGGATTATATTATGACAATAGAAT
TCGCATGTACAGTGAACGAAGAATCACTGTTCTCTACAGCTTAGTTCAAGGACAGCAGTTGAATCCATATTTGAGACTCAAAGTTAGACGTGAC
CATATCATAGATGATGCACTTGTCCGGCTAGAGATGATCGCTATGGAAAATCCTGCAGACTTGAAGAAGCAGTTGTATGTGGAATTTGAAGGAG
AACAAGGAGTTGATGAGGGAGGTGTTTCCAAAGAATTTTTTCAGCTGGTTGTGGAGGAAATCTTCAATCCAGATATTGGTATGTTCACATACGA
TGAATCTACAAAATTGTTTTGGTTTAATCCATCTTCTTTTGAAACTGAGGGTCAGTTTACTCTGATTGGCATAGTACTGGGTCTGGCTATTTAC
AATAACTGTATACTGGATGTACATTTTCCCATGGTTGTCTACAGGAAGCTAATGGGGAAAAAAGGAACTTTTCGTGACTTGGGAGACTCTCACC
CAGTTCTATATCAGAGTTTAAAAGATTTATTGGAGTATGAAGGGAATGTGGAAGATGACATGATGATCACTTTCCAGATATCACAGACAGATCT
TTTTGGTAACCCAATGATGTATGATCTAAAGGAAAATGGTGATAAAATTCCAATTACAAATGAAAACAGGAAGGAATTTGTCAATCTTTATTCT
GACTACATTCTCAATAAATCAGTAGAAAAACAGTTCAAGGCTTTTCGGAGAGGTTTTCATATGGTGACCAATGAATCTCCCTTAAAGTACTTAT
TCAGACCAGAAGAAATTGAATTGCTTATATGTGGAAGCCGGAATCTAGATTTCCAAGCACTAGAAGAAACTACAGAATATGACGGTGGCTATAC
CAGGGACTCTGTTCTGATTAGGGAGTTCTGGGAAATCGTTCATTCATTTACAGATGAACAGAAAAGACTCTTCTTGCAGTTTACAACGGGCACA
GACAGAGCACCTGTGGGAGGACTAGGAAAATTAAAGATGATTATAGCCAAAAATGGCCCAGACACAGAAAGGTTACCTACATCTCATACTTGCT
TTAATGTGCTTTTACTTCCGGAATACTCAAGCAAAGAAAAACTTAAAGAGAGATTGTTGAAGGCCATCACGTATGCCAAAGGATTTGGCATGCT
GTAAAACAAAACAAAACAAAATAAAACAAAAAAAAGGAAGGAAAAAAAAAGAAAAAATTTAAAAAATTTTAAAAATATAACGAGGGATAAATTT
TTGGTGGTGATAGTGTCCCAGTACAAAAAGGCTGTAAGATAGTCAACCACAGTAGTCACCTATGTCTGTGCCTCCCTTCTTTATTGGGGACATG
TGGGCTGGAACAGCAGATTTCAGCTACATATATGAACAAATCCTTTATTATTATTATAATTATTTTTTTGCGTGAAAGTGTTACATATTCTTTC
ACTTGTATGTACAGAGAGGTTTTTCTGAATATTTATTTTAAGGGTTAAATCACTTTTGCTTGTGTTTATTACTGCTTGAGGTTGAGCCTTTTGA
GTATTTAAAAAATATATACCAACAGAACTACTCTCCCAAGGAAAATATTGCCACCATTTGTAGACCACGTAACCTTCAAGTATGTGCTACTTTT
TTGTCCCTGTATCTAACTCAAATCAGGAACTGTATTTTTTTTAATGATTTGCTTTTGAAACTTGAAGTCTTGAAAACAGTGTGATGCAATTACT
GCTGTTCTAGCCCCCAAAGAGTTTTCTGTGCAAAATCTTGAGAATCAATCAATAAAGAAAGATGGAAGGAAGGGAGAAATTGGAATGTTTTAAC
TGCAGCCCTCAGAACTTTAGTAACAGCACAACAAATTAAAAACAAAAACAACTCATGCCACAGTATGTCGTCTTCATGTGTCTTGCAATGAACT
GTTTCAGTAGCCAATCCTCTTTCTTAGTATATGAAAGGACAGGGATTTTTGTTCTTGTTGTTCTCGTTGTTGTTTTAAGTTTACTGGGGAAAGT
GCATTTGGCCAAATGAAATGGTAGTCAAGCCTATTGCAACAAAGTTAGGAAGTTTGTTGTTTGTTTATTATAAACAAAAAGCATGTGAAAGTGC
ACTTAAGATAGAGTTTTTATTAATTACTTACTTATTACCTAGATTTTAAATAGACAATCCAAAGTCTCCCCTTCGTGTTGCCATCATCTTGTTG
AATCAGCCATTTTATCGAGGCACGTGATCAGTGTTGCAACATAATGAAAAAGATGGCTACTGTGCCTTGTGTTACTTAATCATACAGTAAGCTG
ACCTGGAAATGAATGAAACTATTACTCCTAAGAATTACATTGTATAGCCCCACAGATTAAATTTAATTAATTAATTCAAAACATGTTAAACGTT
ACTTTCATGTACTATGGAAAAGTACAAGTAGGTTTACATTACTGATTTCCAGAAGTAAGTAGTTTCCCCTTTCCTAGTCTTCTGTGTATGTGAT
GTTGTTAATTTCTTTTATTGCATTATAAAATAAAAGGATTATGTATTTTTAACTAAGGTGAGACATTGATATATCCTTTTGCTACAAGCTATAG
CTAATGTGCTGAGCTTGTGCCTTGGTGATTGATTGATTGATTGACTGATTGTTTTAACTGATTACTGTAGATCAACCTGATGATTTGTTTGTTT
GAAATTGGCAGGAAAAATGCAGCTTTCAAATCATTGGGGGGAGAAAAAGGATGTCTTTCAGGATTATTTTAATTAATTTTTTTCATAATTGAGA
CAGAACTGTTTGTTATGTACCATAATGCTAAATAAAACTGTGGCACTTTTCACCATAATTTAATTTAGTGGAAAAAGAAGACAATGCTTTCCAT
ATTGTGATAAGGTAACATGGGGTTTTTCTGGGCCAGCCTTTAGAACACTGTTAGGGTACATACGCTACCTTGATGAAAGGGACCTTCGTGCAAC
TGTAGTCATCTTAAAGGCTTCTCATCCACTGTGCTTCTTAATGTGTAATTAAAGTGAGGAGAAATTAAATACTCTGAGGGCGTTTTATATAATA
AATTCGTGAAGA
>gi|332000022|ref|NM_130839.2| Homo sapiens ubiquitin protein ligase E3A (UBE3A), transcript
variant 3, mRNA (SEQ ID NO: 5)
AGCCAGTCCTCCCGTCTTGCGCCGCGGCCGCGAGATCCGTGTGTCTCCCAAGATGGTGGCGCTGGGCTCGGGGTGACTACAGGAGACGACGGGG
CCTTTTCCCTTCGCCAGGACCCGACACACCAGGCTTCGCTCGCTCGCGCACCCCTCCGCCGCGTAGCCATCCGCCAGCGCGGGCGCCCGCCATC
CGCCGCCTACTTACGCTTCACCTCTGCCGACCCGGCGCGCTCGGCTGCGGGCGGCGGCGCCTCCTTCGGCTCCTCCTCGGAATAGCTCGCGGCC
TGTAGCCCCTGGCAGGAGGGCCCCTCAGCCCCCCGGTGTGGACAGGCAGCGGCGGCTGGCGACGAACGCCGGGATTTCGGCGGCCCCGGCGCTC
CCTTTCCCGGCCTCGTTTTCCGGATAAGGAAGCGCGGGTCCCGCATGAGCCCCGGCGGTGGCGGCAGCGAAAGAGAACGAGGCGGTGGCGGGCG
GAGGCGGCGGGCGAGGGCGACTACGACCAGTGAGGCGGCCGCCGCAGCCCAGGCGCGGGGGCGACGACAGGTTAAAAATCTGTAAGAGCCTGAT
TTTAGAATTCACCAGCTCCTCAGAAGTTTGGCGAAATATGAGTTATTAAGCCTACGCTCAGATCAAGGTAGCAGCTAGACTGGTGTGACAACCT
GTTTTTAATCAGTGACTCAAAGCTGTGATCACCCTGATGTCACCGAATGGCCACAGCTTGTAAAAGATCAGGAGAACCTCAGTCTGACGACATT
GAAGCTAGCCGAATGAAGCGAGCAGCTGCAAAGCATCTAATAGAACGCTACTACCACCAGTTAACTGAGGGCTGTGGAAATGAAGCCTGCACGA
ATGAGTTTTGTGCTTCCTGTCCAACTTTTCTTCGTATGGATAATAATGCAGCAGCTATTAAAGCCCTCGAGCTTTATAAGATTAATGCAAAACT
CTGTGATCCTCATCCCTCCAAGAAAGGAGCAAGCTCAGCTTACCTTGAGAACTCGAAAGGTGCCCCCAACAACTCCTGCTCTGAGATAAAAATG
AACAAGAAAGGCGCTAGAATTGATTTTAAAGATGTGACTTACTTAACAGAAGAGAAGGTATATGAAATTCTTGAATTATGTAGAGAAAGAGAGG
ATTATTCCCCTTTAATCCGTGTTATTGGAAGAGTTTTTTCTAGTGCTGAGGCATTGGTACAGAGCTTCCGGAAAGTTAAACAACACACCAAGGA
AGAACTGAAATCTCTTCAAGCAAAAGATGAAGACAAAGATGAAGATGAAAAGGAAAAAGCTGCATGTTCTGCTGCTGCTATGGAAGAAGACTCA
GAAGCATCTTCCTCAAGGATAGGTGATAGCTCACAGGGAGACAACAATTTGCAAAAATTAGGCCCTGATGATGTGTCTGTGGATATTGATGCCA
TTAGAAGGGTCTACACCAGATTGCTCTCTAATGAAAAAATTGAAACTGCCTTTCTCAATGCACTTGTATATTTGTCACCTAACGTGGAATGTGA
CTTGACGTATCACAATGTATACTCTCGAGATCCTAATTATCTGAATTTGTTCATTATCGTAATGGAGAATAGAAATCTCCACAGTCCTGAATAT
CTGGAAATGGCTTTGCCATTATTTTGCAAAGCGATGAGCAAGCTACCCCTTGCAGCCCAAGGAAAACTGATCAGACTGTGGTCTAAATACAATG
CAGACCAGATTCGGAGAATGATGGAGACATTTCAGCAACTTATTACTTATAAAGTCATAAGCAATGAATTTAACAGTCGAAATCTAGTGAATGA
TGATGATGCCATTGTTGCTGCTTCGAAGTGCTTGAAAATGGTTTACTATGCAAATGTAGTGGGAGGGGAAGTGGACACAAATCACAATGAAGAA
GATGATGAAGAGCCCATCCCTGAGTCCAGCGAGCTGACACTTCAGGAACTTTTGGGAGAAGAAAGAAGAAACAAGAAAGGTCCTCGAGTGGACC
CCCTGGAAACTGAACTTGGTGTTAAAACCCTGGATTGTCGAAAACCACTTATCCCTTTTGAAGAGTTTATTAATGAACCACTGAATGAGGTTCT
AGAAATGGATAAAGATTATACTTTTTTCAAAGTAGAAACAGAGAACAAATTCTCTTTTATGACATGTCCCTTTATATTGAATGCTGTCACAAAG
AATTTGGGATTATATTATGACAATAGAATTCGCATGTACAGTGAACGAAGAATCACTGTTCTCTACAGCTTAGTTCAAGGACAGCAGTTGAATC
CATATTTGAGACTCAAAGTTAGACGTGACCATATCATAGATGATGCACTTGTCCGGCTAGAGATGATCGCTATGGAAAATCCTGCAGACTTGAA
GAAGCAGTTGTATGTGGAATTTGAAGGAGAACAAGGAGTTGATGAGGGAGGTGTTTCCAAAGAATTTTTTCAGCTGGTTGTGGAGGAAATCTTC
AATCCAGATATTGGTATGTTCACATACGATGAATCTACAAAATTGTTTTGGTTTAATCCATCTTCTTTTGAAACTGAGGGTCAGTTTACTCTGA
TTGGCATAGTACTGGGTCTGGCTATTTACAATAACTGTATACTGGATGTACATTTTCCCATGGTTGTCTACAGGAAGCTAATGGGGAAAAAAGG
AACTTTTCGTGACTTGGGAGACTCTCACCCAGTTCTATATCAGAGTTTAAAAGATTTATTGGAGTATGAAGGGAATGTGGAAGATGACATGATG
ATCACTTTCCAGATATCACAGACAGATCTTTTTGGTAACCCAATGATGTATGATCTAAAGGAAAATGGTGATAAAATTCCAATTACAAATGAAA
ACAGGAAGGAATTTGTCAATCTTTATTCTGACTACATTCTCAATAAATCAGTAGAAAAACAGTTCAAGGCTTTTCGGAGAGGTTTTCATATGGT
GACCAATGAATCTCCCTTAAAGTACTTATTCAGACCAGAAGAAATTGAATTGCTTATATGTGGAAGCCGGAATCTAGATTTCCAAGCACTAGAA
GAAACTACAGAATATGACGGTGGCTATACCAGGGACTCTGTTCTGATTAGGGAGTTCTGGGAAATCGTTCATTCATTTACAGATGAACAGAAAA
GACTCTTCTTGCAGTTTACAACGGGCACAGACAGAGCACCTGTGGGAGGACTAGGAAAATTAAAGATGATTATAGCCAAAAATGGCCCAGACAC
AGAAAGGTTACCTACATCTCATACTTGCTTTAATGTGCTTTTACTTCCGGAATACTCAAGCAAAGAAAAACTTAAAGAGAGATTGTTGAAGGCC
ATCACGTATGCCAAAGGATTTGGCATGCTGTAAAACAAAACAAAACAAAATAAAACAAAAAAAAGGAAGGAAAAAAAAAGAAAAAATTTAAAAA
ATTTTAAAAATATAACGAGGGATAAATTTTTGGTGGTGATAGTGTCCCAGTACAAAAAGGCTGTAAGATAGTCAACCACAGTAGTCACCTATGT
CTGTGCCTCCCTTCTTTATTGGGGACATGTGGGCTGGAACAGCAGATTTCAGCTACATATATGAACAAATCCTTTATTATTATTATAATTATTT
TTTTGCGTGAAAGTGTTACATATTCTTTCACTTGTATGTACAGAGAGGTTTTTCTGAATATTTATTTTAAGGGTTAAATCACTTTTGCTTGTGT
TTATTACTGCTTGAGGTTGAGCCTTTTGAGTATTTAAAAAATATATACCAACAGAACTACTCTCCCAAGGAAAATATTGCCACCATTTGTAGAC
CACGTAACCTTCAAGTATGTGCTACTTTTTTGTCCCTGTATCTAACTCAAATCAGGAACTGTATTTTTTTTAATGATTTGCTTTTGAAACTTGA
AGTCTTGAAAACAGTGTGATGCAATTACTGCTGTTCTAGCCCCCAAAGAGTTTTCTGTGCAAAATCTTGAGAATCAATCAATAAAGAAAGATGG
AAGGAAGGGAGAAATTGGAATGTTTTAACTGCAGCCCTCAGAACTTTAGTAACAGCACAACAAATTAAAAACAAAAACAACTCATGCCACAGTA
TGTCGTCTTCATGTGTCTTGCAATGAACTGTTTCAGTAGCCAATCCTCTTTCTTAGTATATGAAAGGACAGGGATTTTTGTTCTTGTTGTTCTC
GTTGTTGTTTTAAGTTTACTGGGGAAAGTGCATTTGGCCAAATGAAATGGTAGTCAAGCCTATTGCAACAAAGTTAGGAAGTTTGTTGTTTGTT
TATTATAAACAAAAAGCATGTGAAAGTGCACTTAAGATAGAGTTTTTATTAATTACTTACTTATTACCTAGATTTTAAATAGACAATCCAAAGT
CTCCCCTTCGTGTTGCCATCATCTTGTTGAATCAGCCATTTTATCGAGGCACGTGATCAGTGTTGCAACATAATGAAAAAGATGGCTACTGTGC
CTTGTGTTACTTAATCATACAGTAAGCTGACCTGGAAATGAATGAAACTATTACTCCTAAGAATTACATTGTATAGCCCCACAGATTAAATTTA
ATTAATTAATTCAAAACATGTTAAACGTTACTTTCATGTACTATGGAAAAGTACAAGTAGGTTTACATTACTGATTTCCAGAAGTAAGTAGTTT
CCCCTTTCCTAGTCTTCTGTGTATGTGATGTTGTTAATTTCTTTTATTGCATTATAAAATAAAAGGATTATGTATTTTTAACTAAGGTGAGACA
TTGATATATCCTTTTGCTACAAGCTATAGCTAATGTGCTGAGCTTGTGCCTTGGTGATTGATTGATTGATTGACTGATTGTTTTAACTGATTAC
TGTAGATCAACCTGATGATTTGTTTGTTTGAAATTGGCAGGAAAAATGCAGCTTTCAAATCATTGGGGGGAGAAAAAGGATGTCTTTCAGGATT
ATTTTAATTAATTTTTTTCATAATTGAGACAGAACTGTTTGTTATGTACCATAATGCTAAATAAAACTGTGGCACTTTTCACCATAATTTAATT
TAGTGGAAAAAGAAGACAATGCTTTCCATATTGTGATAAGGTAACATGGGGTTTTTCTGGGCCAGCCTTTAGAACACTGTTAGGGTACATACGC
TACCTTGATGAAAGGGACCTTCGTGCAACTGTAGTCATCTTAAAGGCTTCTCATCCACTGTGCTTCTTAATGTGTAATTAAAGTGAGGAGAAAT
TAAATACTCTGAGGGCGTTTTATATAATAAATTCGTGAAGA
>gi|19718761|ref|NM_130838.1| Homo sapiens ubiquitin protein ligase E3A (UBE3A), transcript
variant 1, mRNA (SEQ ID NO: 6)
ACAGATCAGGAGAACCTCAGTCTGACGACATTGAAGCTAGCCGAATGAAGCGAGCAGCTGCAAAGCATCTAATAGAACGCTACTACCACCAGTT
AACTGAGGGCTGTGGAAATGAAGCCTGCACGAATGAGTTTTGTGCTTCCTGTCCAACTTTTCTTCGTATGGATAATAATGCAGCAGCTATTAAA
GCCCTCGAGCTTTATAAGATTAATGCAAAACTCTGTGATCCTCATCCCTCCAAGAAAGGAGCAAGCTCAGCTTACCTTGAGAACTCGAAAGGTG
CCCCCAACAACTCCTGCTCTGAGATAAAAATGAACAAGAAAGGCGCTAGAATTGATTTTAAAGATGTGACTTACTTAACAGAAGAGAAGGTATA
TGAAATTCTTGAATTATGTAGAGAAAGAGAGGATTATTCCCCTTTAATCCGTGTTATTGGAAGAGTTTTTTCTAGTGCTGAGGCATTGGTACAG
AGCTTCCGGAAAGTTAAACAACACACCAAGGAAGAACTGAAATCTCTTCAAGCAAAAGATGAAGACAAAGATGAAGATGAAAAGGAAAAAGCTG
CATGTTCTGCTGCTGCTATGGAAGAAGACTCAGAAGCATCTTCCTCAAGGATAGGTGATAGCTCACAGGGAGACAACAATTTGCAAAAATTAGG
CCCTGATGATGTGTCTGTGGATATTGATGCCATTAGAAGGGTCTACACCAGATTGCTCTCTAATGAAAAAATTGAAACTGCCTTTCTCAATGCA
CTTGTATATTTGTCACCTAACGTGGAATGTGACTTGACGTATCACAATGTATACTCTCGAGATCCTAATTATCTGAATTTGTTCATTATCGTAA
TGGAGAATAGAAATCTCCACAGTCCTGAATATCTGGAAATGGCTTTGCCATTATTTTGCAAAGCGATGAGCAAGCTACCCCTTGCAGCCCAAGG
AAAACTGATCAGACTGTGGTCTAAATACAATGCAGACCAGATTCGGAGAATGATGGAGACATTTCAGCAACTTATTACTTATAAAGTCATAAGC
AATGAATTTAACAGTCGAAATCTAGTGAATGATGATGATGCCATTGTTGCTGCTTCGAAGTGCTTGAAAATGGTTTACTATGCAAATGTAGTGG
GAGGGGAAGTGGACACAAATCACAATGAAGAAGATGATGAAGAGCCCATCCCTGAGTCCAGCGAGCTGACACTTCAGGAACTTTTGGGAGAAGA
AAGAAGAAACAAGAAAGGTCCTCGAGTGGACCCCCTGGAAACTGAACTTGGTGTTAAAACCCTGGATTGTCGAAAACCACTTATCCCTTTTGAA
GAGTTTATTAATGAACCACTGAATGAGGTTCTAGAAATGGATAAAGATTATACTTTTTTCAAAGTAGAAACAGAGAACAAATTCTCTTTTATGA
CATGTCCCTTTATATTGAATGCTGTCACAAAGAATTTGGGATTATATTATGACAATAGAATTCGCATGTACAGTGAACGAAGAATCACTGTTCT
CTACAGCTTAGTTCAAGGACAGCAGTTGAATCCATATTTGAGACTCAAAGTTAGACGTGACCATATCATAGATGATGCACTTGTCCGGCTAGAG
ATGATCGCTATGGAAAATCCTGCAGACTTGAAGAAGCAGTTGTATGTGGAATTTGAAGGAGAACAAGGAGTTGATGAGGGAGGTGTTTCCAAAG
AATTTTTTCAGCTGGTTGTGGAGGAAATCTTCAATCCAGATATTGGTATGTTCACATACGATGAATCTACAAAATTGTTTTGGTTTAATCCATC
TTCTTTTGAAACTGAGGGTCAGTTTACTCTGATTGGCATAGTACTGGGTCTGGCTATTTACAATAACTGTATACTGGATGTACATTTTCCCATG
GTTGTCTACAGGAAGCTAATGGGGAAAAAAGGAACTTTTCGTGACTTGGGAGACTCTCACCCAGTTCTATATCAGAGTTTAAAAGATTTATTGG
AGTATGAAGGGAATGTGGAAGATGACATGATGATCACTTTCCAGATATCACAGACAGATCTTTTTGGTAACCCAATGATGTATGATCTAAAGGA
AAATGGTGATAAAATTCCAATTACAAATGAAAACAGGAAGGAATTTGTCAATCTTTATTCTGACTACATTCTCAATAAATCAGTAGAAAAACAG
TTCAAGGCTTTTCGGAGAGGTTTTCATATGGTGACCAATGAATCTCCCTTAAAGTACTTATTCAGACCAGAAGAAATTGAATTGCTTATATGTG
GAAGCCGGAATCTAGATTTCCAAGCACTAGAAGAAACTACAGAATATGACGGTGGCTATACCAGGGACTCTGTTCTGATTAGGGAGTTCTGGGA
AATCGTTCATTCATTTACAGATGAACAGAAAAGACTCTTCTTGCAGTTTACAACGGGCACAGACAGAGCACCTGTGGGAGGACTAGGAAAATTA
AAGATGATTATAGCCAAAAATGGCCCAGACACAGAAAGGTTACCTACATCTCATACTTGCTTTAATGTGCTTTTACTTCCGGAATACTCAAGCA
AAGAAAAACTTAAAGAGAGATTGTTGAAGGCCATCACGTATGCCAAAGGATTTGGCATGCTGTAAAACAAAACAAAACAAAATAAAACAAAAAA
AAGGAAGGAAAAAAAAAGAAAAAATTTAAAAAATTTTAAAAATATAACGAGGGATAAATTTTTGGTGGTGATAGTGTCCCAGTACAAAAAGGCT
GTAAGATAGTCAACCACAGTAGTCACCTATGTCTGTGCCTCCCTTCTTTATTGGGGACATGTGGGCTGGAACAGCAGATTTCAGCTACATATAT
GAACAAATCCTTTATTATTATTATAATTATTTTTTTGCGTGAAAGTGTTACATATTCTTTCACTTGTATGTACAGAGAGGTTTTTCTGAATATT
TATTTTAAGGGTTAAATCACTTTTGCTTGTGTTTATTACTGCTTGAGGTTGAGCCTTTTGAGTATTTAAAAAATATATACCAACAGAACTACTC
TCCCAAGGAAAATATTGCCACCATTTGTAGACCACGTAACCTTCAAGTATGTGCTACTTTTTTGTCCCTGTATCTAACTCAAATCAGGAACTGT
ATTTTTTTTAATGATTTGCTTTTGAAACTTGAAGTCTTGAAAACAGTGTGATGCAATTACTGCTGTTCTAGCCCCCAAAGAGTTTTCTGTGCAA
AATCTTGAGAATCAATCAATAAAGAAAGATGGAAGGAAGGGAGAAATTGGAATGTTTTAACTGCAGCCCTCAGAACTTTAGTAACAGCACAACA
AATTAAAAACAAAAACAACTCATGCCACAGTATGTCGTCTTCATGTGTCTTGCAATGAACTGTTTCAGTAGCCAATCCTCTTTCTTAGTATATG
AAAGGACAGGGATTTTTGTTCTTGTTGTTCTCGTTGTTGTTTTAAGTTTACTGGGGAAAGTGCATTTGGCCAAATGAAATGGTAGTCAAGCCTA
TTGCAACAAAGTTAGGAAGTTTGTTGTTTGTTTATTATAAACAAAAAGCATGTGAAAGTGCACTTAAGATAGAGTTTTTATTAATTACTTACTT
ATTACCTAGATTTTAAATAGACAATCCAAAGTCTCCCCTTCGTGTTGCCATCATCTTGTTGAATCAGCCATTTTATCGAGGCACGTGATCAGTG
TTGCAACATAATGAAAAAGATGGCTACTGTGCCTTGTGTTACTTAATCATACAGTAAGCTGACCTGGAAATGAATGAAACTATTACTCCTAAGA
ATTACATTGTATAGCCCCACAGATTAAATTTAATTAATTAATTCAAAACATGTTAAACGTTACTTTCATGTACTATGGAAAAGTACAAGTAGGT
TTACATTACTGATTTCCAGAAGTAAGTAGTTTCCCCTTTCCTAGTCTTCTGTGTATGTGATGTTGTTAATTTCTTTTATTGCATTATAAAATAA
AAGGATTATGTATTTTTAACTAAGGTGAGACATTGATATATCCTTTTGCTACAAGCTATAGCTAATGTGCTGAGCTTGTGCCTTGGTGATTGAT
TGATTGATTGACTGATTGTTTTAACTGATTACTGTAGATCAACCTGATGATTTGTTTGTTTGAAATTGGCAGGAAAAATGCAGCTTTCAAATCA
TTGGGGGGAGAAAAAGGATGTCTTTCAGGATTATTTTAATTAATTTTTTTCATAATTGAGACAGAACTGTTTGTTATGTACCATAATGCTAAAT
AAAACTGTGGCACTTTTCACCATAATTTAATTTAGTGGAAAAAGAAGACAATGCTTTCCATATTGTGATAAGGTAACATGGGGTTTTTCTGGGC
CAGCCTTTAGAACACTGTTAGGGTACATACGCTACCTTGATGAAAGGGACCTTCGTGCAACTGTAGTCATCTTAAAGGCTTCTCATCCACTGTG
CTTCTTAATGTGTAATTAAAGTGAGGAGAAATTAAATACTCTGAGGGCGTTTTATATAATAAATTCGTGAAGA

Protein sequences of ube3a are also well known to those of skill in the art. Some exemplary ube3a sequences are given below, however, it should be appreciated that the invention is not limited to these specific sequences and that additional ube3a protein sequences are known to those of skill in the art.

>gi|76880500|ref|NP_001029134.1| ubiquitin-protein ligase E3A isoform 3 [Mus musculus] (SEQ ID
NO: 7)
MKRAAAKHLIERYYHQLTEGCGNEACTNEFCASCPTFLRMDNNAAAIKALELYKINAKLCDPHPSKKGASSAYLENSKGASNNSEIKMNKKEGK
DFKDVIYLTEEKVYEIYEFCRESEDYSPLIRVIGRIFSSAEALVLSFRKVKQHTKEELKSLQEKDEDKDEDEKEKAACSAAAMEEDSEASSSRM
GDSSQGDNNVQKLGPDDVTVDIDAIRRVYSSLLANEKLETAFLNALVYLSPNVECDLTYHNVYTRDPNYLNLFIIVMENSNLHSPEYLEMALPL
FCKAMCKLPLEAQGKLIRLWSKYSADQIRAMMETFQQLITYKVISNEFNSRNLVNDDDAIVAASKCLKMVYYANVVGGDVDTNHNEEDDEEPIP
ESSELTLQELLGDERRNKKGPRVDPLETELGVKTLDCRKPLISFEEFINEPLNDVLEMDKDYTFFKVETENKFSFMTCPFILNAVTKNLGLYYD
NRIRMYSERRITVLYSLVQGQQLNPYLRLKVRRDHIIDDALVRLEMIAMENPADLKKQLYVEFEGEQGVDEGGVSKEFFQLVVEEIFNPDIGMF
TYDEATKLFWFNPSSFETEGQFTLIGIVLGLAIYNNCILDVHFPMVVYRKLMGKKGTFRDLGDSHPVLYQSLKDLLEYEGSVEDDMMITFQISQ
TDLFGNPMMYDLKENGDKIPITNENRKEFVNLYSDYILNKSVEKQFKAFRRGFHMVTNESPLKYLFRPEEIELLICGSRNLDFQALEETTEYDG
GYTRESVVIREFWEIVHSFTDEQKRLFLQFTTGTDRAPVGGLGKLKMIIAKNGPDTERLPTSHTCFNVLLLPEYSSKEKLKERLLKAITYAKGF
GML
>gi|76880494|ref|NP_035798.2| ubiquitin-protein ligase E3A isoform 2 [Mus musculus] (SEQ ID
NO: 8)
MATACKRSPGESQSEDIEASRMKRAAAKHLIERYYHQLTEGCGNEACTNEFCASCPTFLRMDNNAAAIKALELYKINAKLCDPHPSKKGASSAY
LENSKGASNNSEIKMNKKEGKDFKDVIYLTEEKVYEIYEFCRESEDYSPLIRVIGRIFSSAEALVLSFRKVKQHTKEELKSLQEKDEDKDEDEK
EKAACSAAAMEEDSEASSSRMGDSSQGDNNVQKLGPDDVTVDIDAIRRVYSSLLANEKLETAFLNALVYLSPNVECDLTYHNVYTRDPNYLNLF
IIVMENSNLHSPEYLEMALPLFCKAMCKLPLEAQGKLIRLWSKYSADQIRRMMETFQQLITYKVISNEFNSRNLVNDDDAIVAASKCLKMVYYA
NVVGGDVDTNHNEEDDEEPIPESSELTLQELLGDERRNKKGPRVDPLETELGVKTLDCRKPLISFEEFINEPLNDVLEMDKDYTFFKVETENKF
SFMTCPFILNAVTKNLGLYYDNRIRMYSERRITVLYSLVQGQQLNPYLRLKVARDHIIDDALVALEMIAMENPADLKKQLYVEFEGEQGVDEGG
VSKEFFQLVVEEIFNPDIGMFTYDEATKLFWFNPSSFETEGQFTLIGIVLGLAIYNNCILDVHFPMVVYRKLMGKKGTFRDLGDSHPVLYQSLK
DLLEYEGSVEDDMMITFQISQTDLFGNPMMYDLKENGDKIPITNENRKEFVNLYSDYILNKSVEKQFKAFRRGFHMVTNESPLKYLFRPEEIEL
LICGSRNLDFQALEETTEYDGGYTRESVVIREFWEIVHSFTDEQKRLFLQFTTGTDRAPVGGLGKLKMIIAKNGPDTERLPTSHTCFNVLLLPE
YSSKEKLKERLLKAITYAKGFGML
>gi|27804321|ref|NP_766598.1| ubiquitin-protein ligase E3A isoform 1 [Mus musculus] (SEQ ID
NO: 9)
MKRAAAKHLIERYYHQLTEGCGNEACTNEFCASCPTFLRMDNNAAAIKALELYKINAKLCDPHPSKKGASSAYLENSKGASNNSEIKMNKKEGK
DFKDVIYLTEEKVYEIYEFCRESEDYSPLIRVIGRIFSSAEALVLSFRKVKQHTKEELKSLQEKDEDKDEDEKEKAACSAAAMEEDSEASSSRM
GDSSQGDNNVQKLGPDDVTVDIDAIRRVYSSLLANEKLETAFLNALVYLSPNVECDLTYHNVYTRDPNYLNLFIIVMENSNLHSPEYLEMALPL
FCKAMCKLPLEAQGKLIRLWSKYSADQIRRMMETFQQLITYKVISNEFNSRNLVNDDDAIVAASKCLKMVYYANVVGGDVDTNHNEEDDEEPIP
ESSELTLQELLGDERRNKKGPRVDPLETELGVKTLDCRKPLISFEEFINEPLNDVLEMDKDYTFFKVETENKFSFMTCPFILNAVTKNLGLYYD
NRIRMYSERRITVLYSLVQGQQLNPYLRLKVARDHIIDDALVALEMIAMENPADLKKQLYVEFEGEQGVDEGGVSKEFFQLVVEEIFNPDIGMF
TYDEATKLFWFNPSSFETEGQFTLIGIVLGLAIYNNCILDVHFPMVVYRKLMGKKGTFRDLGDSHPVLYQSLKDLLEYEGSVEDDMMITFQISQ
TDLFGNPMMYDLKENGDKIPITNENRKEFVNLYSDYILNKSVEKQFKAFRRGFHMVTNESPLKYLFRPEEIELLICGSRNLDFQALEETTEYDG
GYTRESVVIR
>gi|19718766|ref|NP_000453.2| ubiquitin-protein ligase E3A isoform 2 [Homo sapiens] (SEQ ID
NO: 10)
MEKLHQCYWKSGEPQSDDIEASRMKRAAAKHLIERYYHQLTEGCGNEACTNEFCASCPTFLRMDNNAAAIKALELYKINAKLCDPHPSKKGASS
AYLENSKGAPNNSCSEIKMNKKGARIDFKDVTYLTEEKVYEILELCREREDYSPLIRVIGRVFSSAEALVQSFRKVKQHTKEELKSLQAKDEDK
DEDEKEKAACSAAAMEEDSEASSSRIGDSSQGDNNLQKLGPDDVSVDIDAIRRVYTRLLSNEKIETAFLNALVYLSPNVECDLTYHNVYSRDPN
YLNLFIIVMENRNLHSPEYLEMALPLFCKAMSKLPLAAQGKLIRLWSKYNADQIRRMMETFQQLITYKVISNEFNSRNLVNDDDAIVAASKCLK
MVYYANVVGGEVDTNHNEEDDEEPIPESSELTLQELLGEERRNKKGPRVDPLETELGVKTLDCRKPLIPFEEFINEPLNEVLEMDKDYTFEKVE
TENKFSFMTCPFILNAVTKNLGLYYDNRIRMYSERRITVLYSLVQGQQLNPYLRLKVRRDHIIDDALVALEMIAMENPADLKKQLYVEFEGEQG
VDEGGVSKEFFQLVVEEIFNPDIGMFTYDESTKLFWFNPSSFETEGQFTLIGIVLGLAIYNNCILDVHFPMVVYRKLMGKKGTFRDLGDSHPVL
YQSLKDLLEYEGNVEDDMMITFQISQTDLFGNPMMYDLKENGDKIPITNENRKEFVNLYSDYILNKSVEKQFKAFRRGFHMVTNESPLKYLFRP
EEIELLICGSRNLDFQALEETTEYDGGYTRDSVLIREFWEIVHSFTDEQKRLFLQFTTGTDRAPVGGLGKLKMIIAKNGPDTERLPTSHTCFNV
LLLPEYSSKEKLKERLLKAITYAKGEGML
>gi|19718764|ref|NP_570854.1| ubiquitin-protein ligase E3A isoform 3 [Homo sapiens] (SEQ ID
NO: 11)
MATACKRSGEPQSDDIEASRMKRAAAKHLIERYYHQLTEGCGNEACTNEFCASCPTFLRMDNNAAAIKALELYKINAKLCDPHPSKKGASSAYL
ENSKGAPNNSCSEIKMNKKGARIDFKDVTYLTEEKVYEILELCREREDYSPLIRVIGRVFSSAEALVQSFRKVKQHTKEELKSLQAKDEDKDED
EKEKAACSAAAMEEDSEASSSRIGDSSQGDNNLQKLGPDDVSVDIDAIRRVYTALLSNEKIETAFLNALVYLSPNVECDLTYHNVYSRDPNYLN
LFIIVMENRNLHSPEYLEMALPLFCKAMSKLPLAAQGKLIRLWSKYNADQIRRMMETFQQLITYKVISNEFNSRNLVNDDDAIVAASKCLKMVY
YANVVGGEVDTNHNEEDDEEPIPESSELTLQELLGEERRNKKGPRVDPLETELGVKTLDCRKPLIPFEEFINEPLNEVLEMDKDYTFFKVETEN
KFSFMTCPFILNAVTKNLGLYYDNRIRMYSERRITVLYSLVQGQQLNPYLRLKVERDHIIDDALVRLEMIAMENPADLKKQLYVEFEGEQGVDE
GGVSKEFFQLVVEEIFNPDIGMFTYDESTKLFWFNPSSFETEGQFTLIGIVLGLAIYNNCILDVHFPMVVYRKLMGKKGTFRDLGDSHPVLYQS
LKDLLEYEGNVEDDMMITFQISQTDLFGNPMMYDLKENGDKIPITNENRKEFVNLYSDYILNKSVEKQFKAFRRGFHMVTNESPLKYLFRPEEI
ELLICGSRNLDFQALEETTEYDGGYTRDSVLIREFWEIVHSFTDEQKRLFLQFTTGTDRAPVGGLGKLKMIIAKNGPDTERLPTSHTCFNVLLL
PEYSSKEKLKERLLKAITYAKGFGML
>gi|19718762|ref|NP_570853.1| ubiquitin-protein ligase E3A isoform 1 [Homo sapiens] (SEQ ID
NO: 12)
MKRAAAKHLIERYYHQLTEGCGNEACTNEFCASCPTFLAMDNNAAAIKALELYKINAKLCDPHPSKKGASSAYLENSKGAPNNSCSEIKMNKKG
ARIDFKDVTYLTEEKVYEILELCREREDYSPLIRVIGRVFSSAEALVQSFRKVKQHTKEELKSLQAKDEDKDEDEKEKAACSAAAMEEDSEASS
SRIGDSSQGDNNLQKLGPDDVSVDIDAIRRVYTRLLSNEKIETAFLNALVYLSPNVECDLTYHNVYSRDPNYLNLFIIVMENRNLHSPEYLEMA
LPLFCKAMSKLPLAAQGKLIRLWSKYNADQIRRMMETFQQLITYKVISNEFNSRNLVNDDDAIVAASKCLKMVYYANVVGGEVDTNHNEEDDEE
PIPESSELTLQELLGEERRNKKGPRVDPLETELGVKTLDCRKPLIPFEEFINEPLNEVLEMDKDYTFFKVETENKFSFMTCPFILNAVTKNLGL
YYDNRIRMYSERRITVLYSLVQGQQLNPYLRLKVRRDHIIDDALVRLEMIAMENPADLKKQLYVEFEGEQGVDEGGVSKEFFQLVVEEIFNPDI
GMFTYDESTKLFWFNPSSFETEGQFTLIGIVLGLAIYNNCILDVHFPMVVYRKLMGKKGTFRDLGDSHPVLYQSLKDLLEYEGNVEDDMMITFQ
ISQTDLFGNPMMYDLKENGDKIPITNENRKEFVNLYSDYILNKSVEKQFKAFRRGFHMVTNESPLKYLFRPEEIELLICGSRNLDFQALEETTE
YDGGYTRDSVLIREFWEIVHSFTDEQKRLFLQFTTGTDRAPVGGLGKLKMIIAKNGPDTERLPTSHTCFNVLLLPEYSSKEKLKERLLKAITYA
KGFGML

The entire contents of all database entries listed above are incorporated herein by reference.

In some embodiments, a transgenic mammal or transgenic mammalian cell is provided that can be used as a model for autism, wherein the mammal or the cell comprises, in its genome, an additional copy, or additional copies of a wild-type mammalian ube3a gene, e.g., a human or mouse ube3a gene. A wild-type ube3a gene, as used herein, is a genomic region found in a wild type mammal, wherein the genomic region comprises a ube3a coding sequence, typically a sequence comprising introns and exons, and a ube3a promoter. In some embodiments, a transgenic mammal or transgenic mammalian cell includes one or more isolated nucleic acid sequence(s) encoding a ube3a protein comprise a fragment of mouse chromosome 7. Wild type ube3a genes and sequences are well known to those of skill in the art. In some embodiments, the wild-type genomic region encoding ube3a comprises a fragment of the mouse or the human ube3a gene, for example, as described in the following NCBI database entries, the entire contents of which are incorporated herein by reference.

In humans, the ube3a gene is located on chromosome 15:
Official Symbol: UBE3A and Name: ubiquitin protein ligase E3A [Homo sapiens]

### Other Aliases: ANCR, AS, E6-AP, EPVE6AP, FLJ26981, HPVE6A

Other Designations: CTCL tumor antigen se37-2; E6AP ubiquitin-protein ligase; human papilloma virus E6-associated protein; human papillomavirus E6-associated protein; oncogenic protein-associated protein E6-AP; renal carcinoma antigen NY-REN-54; ubiquitin-protein ligase E3A

### Chromosome: 15; Location: 15q11.2

Annotation: Chromosome 15, NC_{—}000015.9 (25582396 . . . 25684128, complement)

### MIM: 601623

### ID: 7337

In mouse, the ube3a gene is located on Chromosome 7:
Official Symbol: Ube3a and Name: ubiquitin protein ligase E3A [Mus musculus]
Other Aliases: 4732496802, 5830462NO2Rik, A130086L21Rik, Hpve6a, KIAA4216, mKIAA4216
Other Designations: E6-AP ubiquitin protein ligase; oncogenic protein-associated protein E6-AP; ubiquitin conjugating enzyme E3A; ubiquitin-protein ligase E3A

### Chromosome: 7; Location: 7 28.65 cM

### Annotation: Chromosome 7, NC_{—}000073.5 (66484120 . . . 66562097)

### ID: 22215

In some embodiments, a transgenic mammal or transgenic mammalian cell is provided herein that comprises an additional copy of a ube3a gene, wherein the additional copy of the ube3a gene comprises a genomic fragment of the ube3a genomic region (also referred to as the ube3a gene locus), for example, of mouse chromosome 7 or of human chromosome 15, of about 50 kb, about 60 kb, about 80 kb, about 90 kb, about 100 kb, about 110 kb, about 120 kb, about 130 kb, about 140 kb, about 150 kb, about 160 kb, about 170 kb, about 180 kb, about 190 kb, about 200 kb, or more than about 200 kb. In some embodiments, the fragment comprises about 162 kb of the ube3a genomic region. In some embodiments, the fragment comprises the entire exon-intron coding sequence of ube3a. The location and sequence of ube3a introns and exons of a given ube3a gene locus, for example, the human ube3a gene locus on chromosome 15 or the mouse locus on chromosome 7, are well known to those of skill in the art. In some embodiments, the fragment comprising the exon-intron coding sequence of ube3a is about 78 kb long.

In some embodiments, the fragment comprises a nucleic acid sequence located 5′ (upstream) of the ube3a genomic region encoding the ube3a transcript. In some embodiments, the upstream region comprises the ube3a promoter region. In some embodiments, the ube3a genomic region fragment comprises at least about 1 kb, at least about 2 kb, at least about 3 kb, at least about 4 kb, at least about 5 kb, at least about 10 kb, at least about 20 kb, at least about 25 kb, at least about 30 kb, at least about 40 kb, at least about 50 kb, at least about 60 kb, at least about 70 kb, at least about 80 kb, at least about 90 kb, or at least about 100 kb of the mouse chromosome 7 region or of the human chromosome 15 region immediately upstream (5′) of the exon-intron coding sequence of ube3a. In some embodiment, the ube3a genomic fragment comprises about 63 kb of the mouse chromosome 7 region or the human chromosome 15 region immediately upstream (5′) of the exon-intron coding sequence of ube3a.

In some embodiment, the additional genomic ube3a fragment further comprises a 3′ (downstream) sequence, for example, a sequence that lies immediately downstream of the region encoding the ube3a transcript. In some embodiments, the 3′ region comprises regulatory elements. In some embodiments, the additional genomic ube3a fragment comprising at least about 1 kb, at least about 2 kb, at least about 3 kb, at least about 4 kb, at least about 5 kb, at least about 10 kb, at least about 20 kb, at least about 25 kb, at least about 30 kb, at least about 40 kb, at least about 50 kb, at least about 60 kb, at least about 70 kb, at least about 80 kb, at least about 90 kb, or at least about 100 kb of the mouse chromosome 7 region or the human chromosome 15 region immediately downstream (3′) of the exon-intron coding sequence of ube3a. In some embodiments, the genomic ube3a fragment comprises about 21 kb of the chromosome 7 region or of the chromosome 15 region immediately downstream (3′) of the exon-intron coding sequence of ube3a.

In some embodiments, the additional copy of a ube3a encoding nucleic acid sequence further comprises a nucleic acid sequence encoding a tag. In some embodiments, the nucleic acid sequence encoding the tag is in frame with the ube3a encoding sequence so that a fusion protein is encoded, for example, a C-terminally or an N-terminally tagged ube3a protein. In some embodiments, the tag is a FLAG tag, a poly-histidine tag (e.g., a 6His tag), or a GST tag. Additional tags are known to those of skill in the art and the invention is not limited in this respect. The use of tags allows for the identification of cells expressing the additional ube3a copy, and for the recovery of exogenous ube3a from the expressing cells via a binding agent specifically binding the tag.

In some embodiments, transcription of the additional copy of a nucleic acid encoding ube3a comprised in the transgenic cell or mammal is driven by a ube3a promoter, for example, a wild-type ube3a promoter. For example, this is the case in some embodiments, where the additional copy of ube3a is introduced into the transgenic animal or cell as a genomic fragment of a ube3a gene comprising both the ube3a-encoding region and at least a fragment of the 5′ region comprising the ube3a promoter. In some embodiments, transcription of the additional copy of ube3a is driven by a heterologous promoter. A heterologous promoter is a promoter that is not naturally operably linked to the specific gene it is driving in an artificial gene expression construct. For example, a constitutive promoter such as a CAGS promoter, a ubiquitinC promoter, or a CMV promoter could be used as heterologous promoters to drive transcription of the additional copy of ube3a in a cell or animal provided herein. Or a human ube3a promoter could be used to drive expression of a mouse ube3a-encoding genomic fragment. Other suitable heterologous constitutive promoters will be apparent to those of skill in the art as the invention is not limited in this respect. In some embodiments, the heterologous promoter is a cell-type specific or a tissue-specific promoter. For example, in some embodiments, the promoter is a neuronal cell specific, brain specific, neuron-specific or glial cell-specific promoter, for example, a tau promoter, CaM-kinase promoter, nestin-promoter, GFAP promoter, tubulin HI promoter, or other promoter known by those of skill in the art to be active specifically in one of these cell types or tissues.

In some embodiments, transcription of the additional copy of ube3a is driven by a heterologous, inducible promoter. Inducible promoters are well-known to those of skill in the art and include, for example, drug inducible promoters, such a tetracycline and tamoxifen-inducible promoters, and recombination-inducible-promoters, such as promoters that become active upon excision of a spacer fragment by cre recombinase. Such inducible promoters allow for the expression of the additional copy of ube3a in only a restricted number of cells, cell types, or tissues, for example, only in neurons, wile the transgene is silent or essentially silent in most or all other cell types or tissues of the transgenic animal. Suitable inducible promoters for a specific cell type will be apparent to those of skill in the art.

In some embodiment, a transgenic non-human mammal is provided that comprises a cell comprising one or more extra copies, in addition to any endogenous copies, of a ube3a encoding nucleic acid sequence. In some embodiments, the mammal comprises at least one cell having a genetic modification as described herein, or is derived from such a cell (e.g. an embryonic stem cell) comprising such a modification. In some embodiments, at least one germ cell of the mammal, and in some embodiments, all cells of the mammal comprise the genetic modification. In some embodiments, the transgenic non-human mammal is a mouse. In some embodiments, the transgenic non-human mammal exhibits at least one phenotypic trait found in autism, for example, (i) impaired social interaction; (ii) defective communication (e.g., vocalization); and/or (iii) repetitive behavior (e.g., self-grooming). In some embodiments, the transgenic mammal exhibits all of these three traits.

Some aspects of this invention provide methods of using the transgenic cells and mammals described herein, for example, in the analysis of pathophysiological mechanisms underlying autism and in the identification of agents that ameliorate the pathological status observed in the transgenic cells or animals. For example, in some embodiments, a transgenic animal, cell, or animal model, is used to identify the molecular basis for the pathological alterations or abberations observed in such cells. Some such pathophysiological characteristics that can be observed in some of the transgenic cells and animals are described herein, and other such characteristics can be observed or measured by those of skill in the art without more than routine experimentation. Such pathological characteristics include, but are not limited to (A) phenotypic/behavioural characteristics, such as (i) impaired social interaction, (ii) reduced communication (vocalization), and/or (iii) increased repetitive, stereotyped behaviors (e.g., grooming), (B) cellular/molecular characteristics, such as reduced or impaired glutamatergic synaptic transmission, reduced/impaired presynaptic glutamate release, and/or reduced/impaired postsynaptic excitability to phasic synapse-like stimuli. Assays for measuring these characteristics are described herein and additional assays and methods suitable for measuring such characteristics will be apparent to those of skill in the art. For example, in some embodiments, a candidate agent is administered to a transgenic animal provided herein that shows a pathological characteristic associated with autism, such as impaired social behavior, or reduced glutamatergic synaptic transmission. The animal is then assessed after a period of time has passed, for example, a time period that is or is believed to be sufficient for the candidate drug to effect an amelioration of the pathological characteristic observed in the animal. If an amelioration of a pathological characteristic associated with autism is observed in the treated animal, for example an improvement of social behavior, or an increase in glutamatergic synapse transmission, then the drug is identified as a candidate drug for the treatment of autism or an autism spectrum disorder. Similarly, a transgenic cell provided herein, for example, a neuronal cell comprising an elevated ube3a gene copy number, can be contacted with a candidate drug and subsequently be assessed for an improvement of a pathological characteristic in response to the drug.

In some embodiments, autism diagnostic methods that are based on the measurement of ube3a protein or activity levels in a subject are provided. For example, in some embodiments, a method of diagnosing an increased risk of developing autism or an autism spectrum disorder in a subject is provided. In some embodiments, the method comprises determining a level of a ube3a protein or of ube3a activity in a sample obtained from the subject. Assays suitable for detecting the level of expression of ube3a or of ube3a activity in a sample are descried herein and additional suitable assays will be apparent to those of skill in the art. It should be understood that the invention is not limited in this respect. In some embodiments, the method further comprises comparing the level or the activity of ube3a determined in the subject to a control or reference level. A control or reference level can be, in some embodiments, a level observed or expected in healthy subjects, for example, in healthy subjects that are age- and sex-matched to the subject in question. In other embodiments, the reference or control level is based on historical data, for example, an average of ube3a protein levels or activity levels observed in a population of subjects. In some embodiments, the control or reference level is based on a sample obtained from a healthy individual that is run side-by-side through the assay used to determine the ube3a level or activity in the sample obtained from the subject. In some embodiments, if the level of ube3a protein detected in the subject is higher than the control or reference level, the subject is identified as a subject at an increased risk of developing autism or an autism spectrum disorder. In some embodiments, the method further comprises initiating health care appropriate to address one or more of the clinical manifestations of autism in response to an increased risk of developing autism in the subject.

The function and advantage of these and other embodiments of the present invention will be more fully understood from the examples below. The following examples are intended to illustrate the benefits of the present invention, but do not exemplify the full scope of the invention.

### Examples

### Selectively Increasing the 15q11-13 Gene Ube3a Copy Number

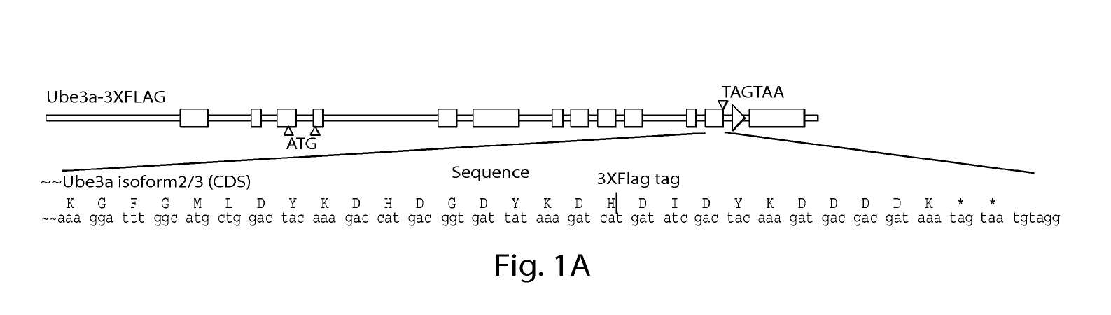

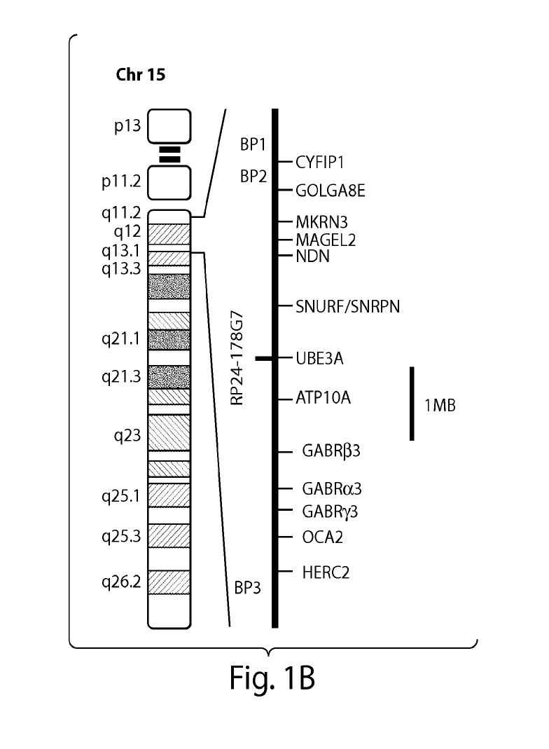

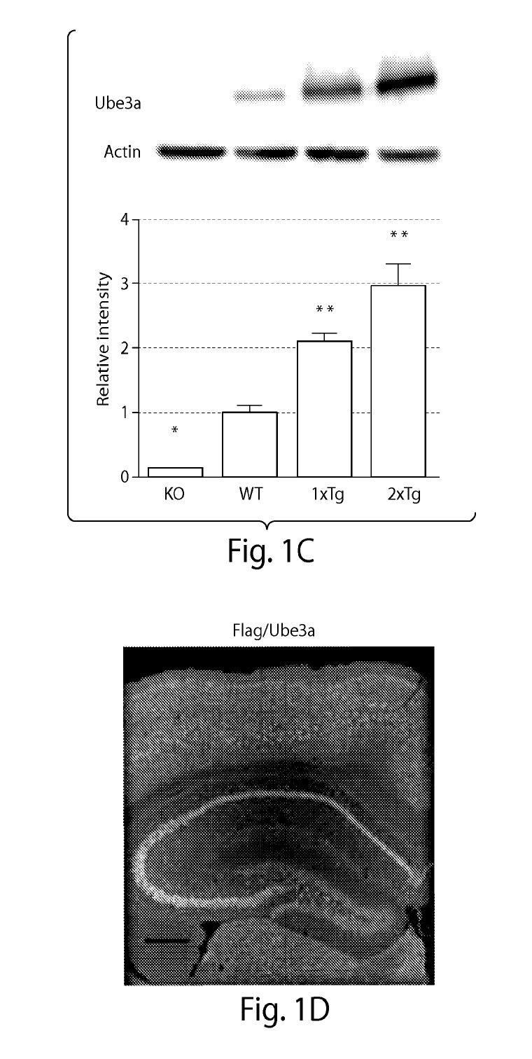

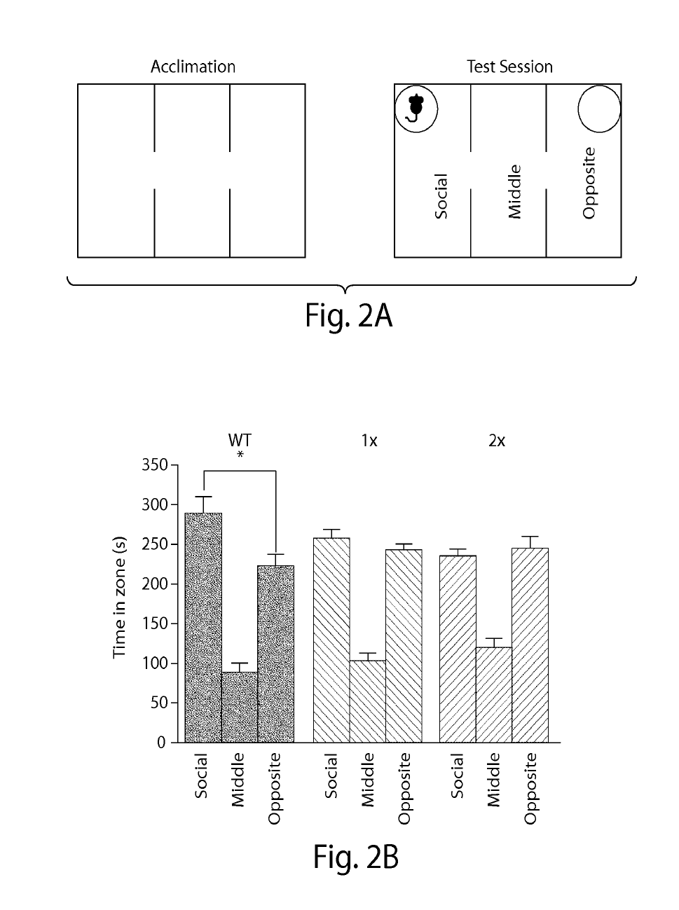

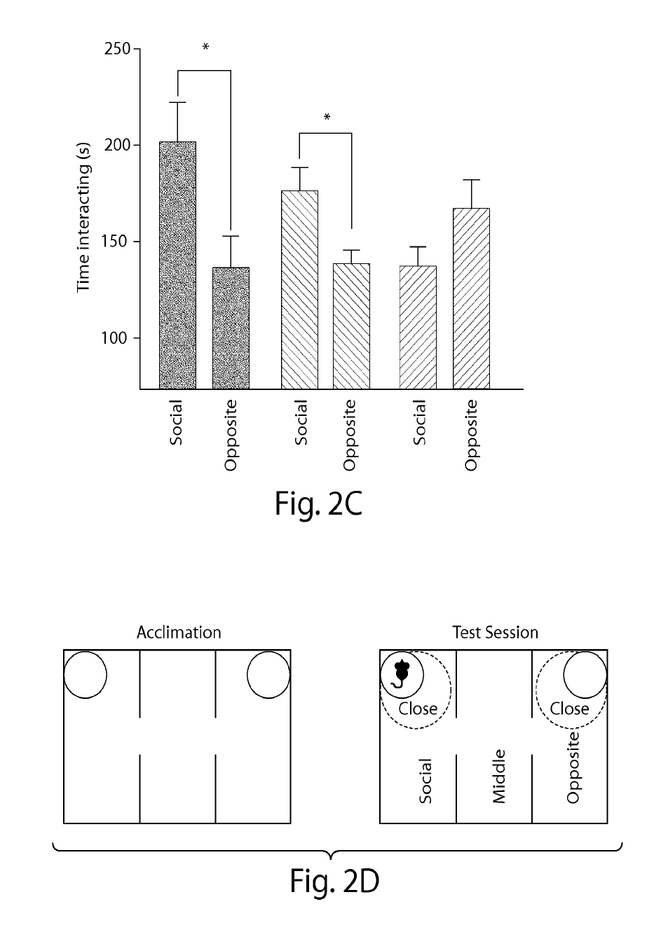

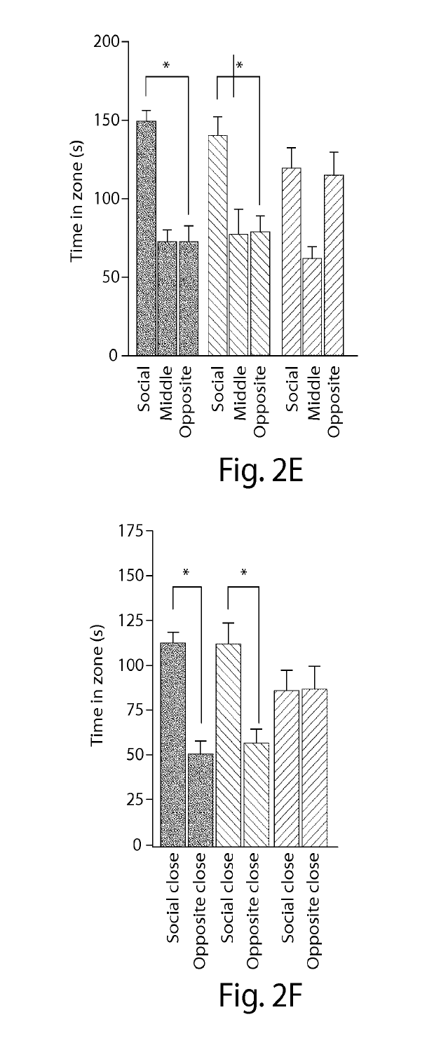

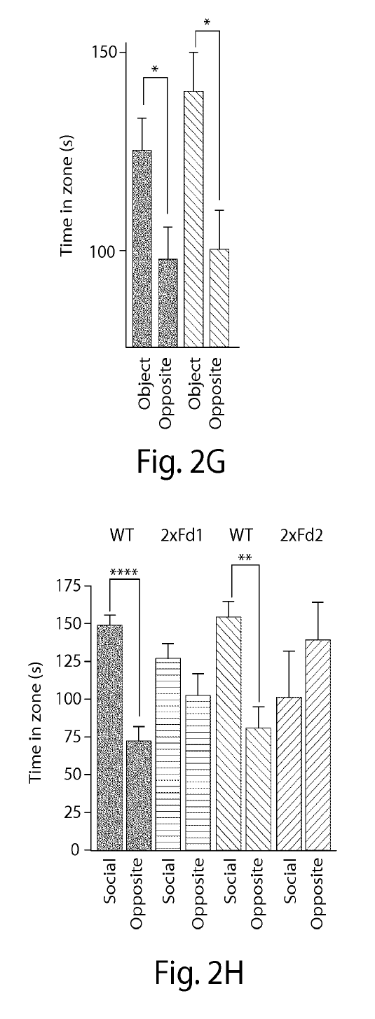

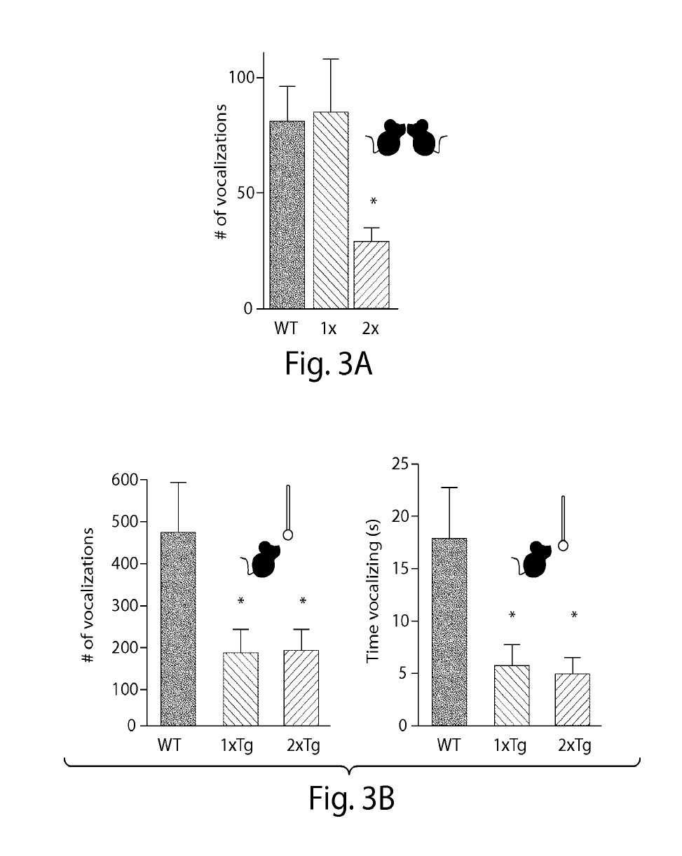

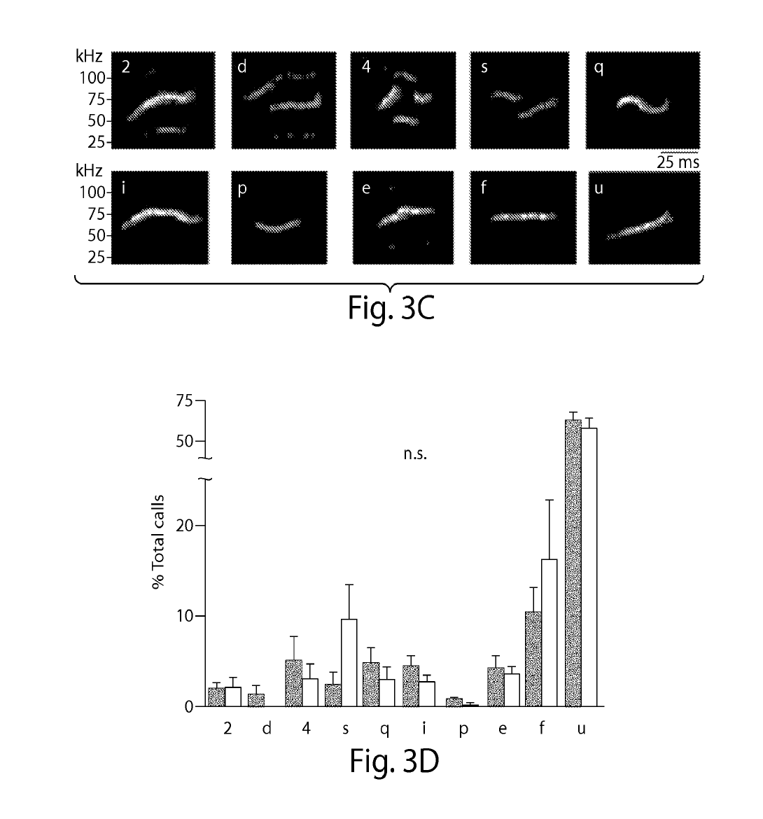

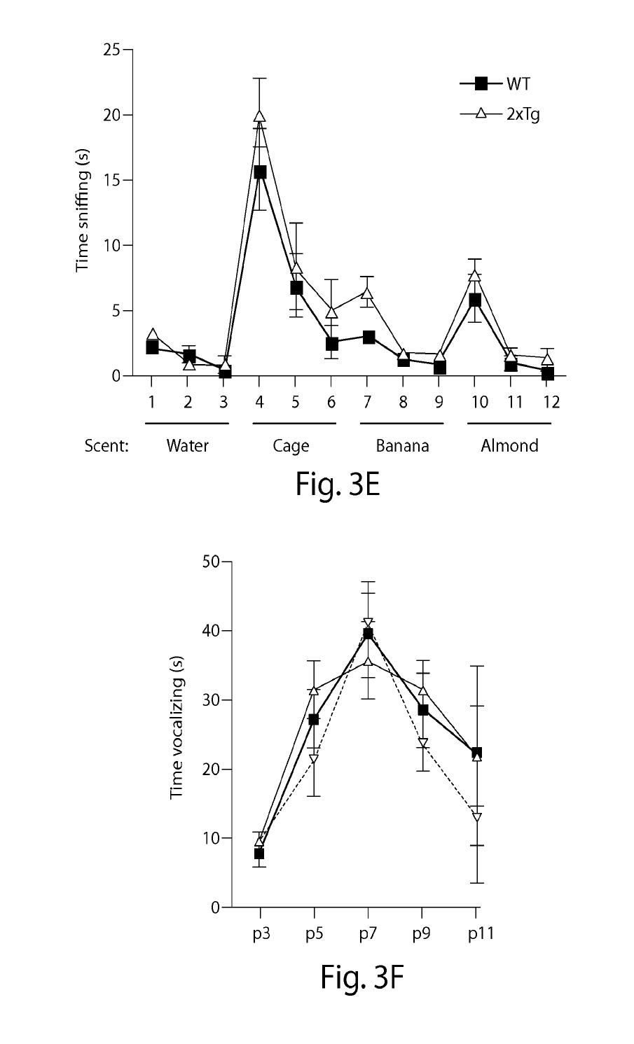

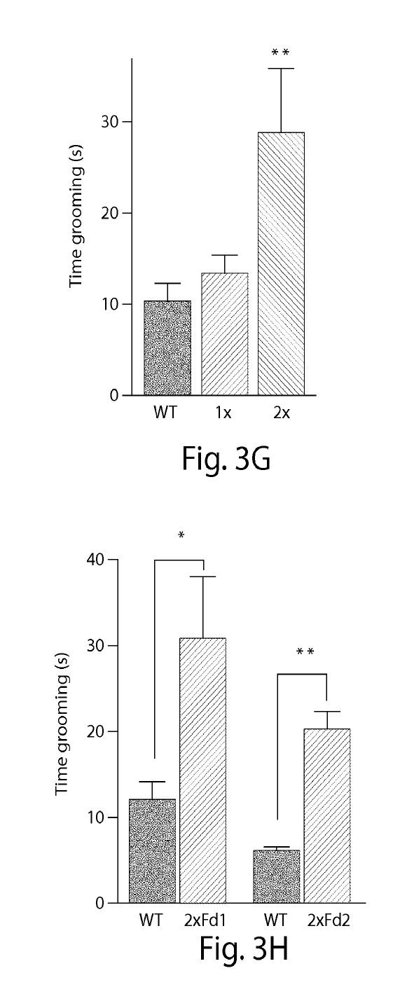

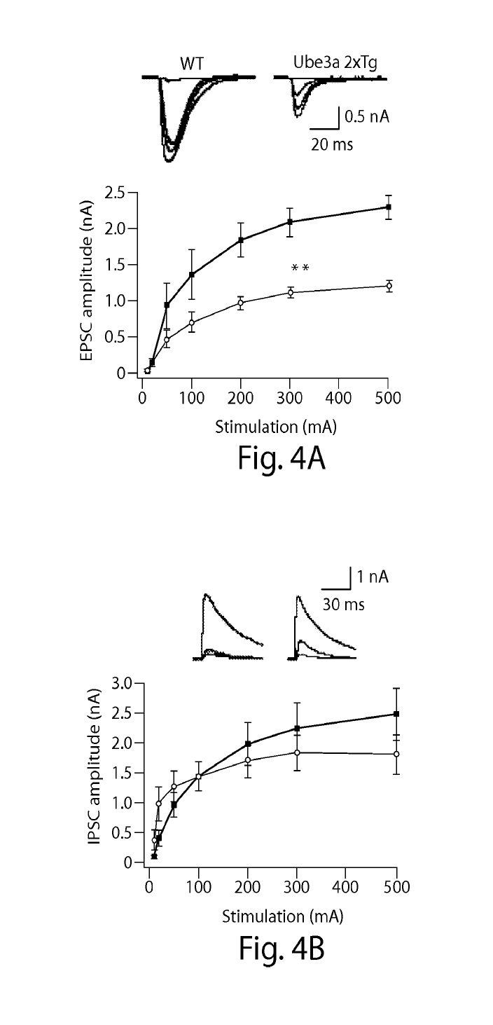

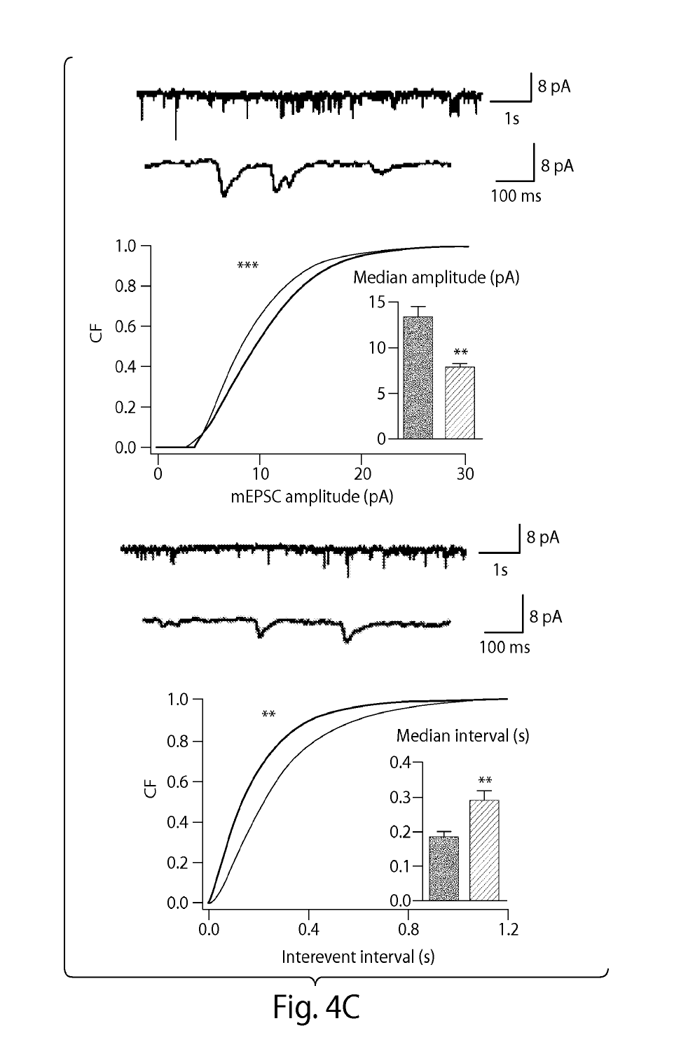

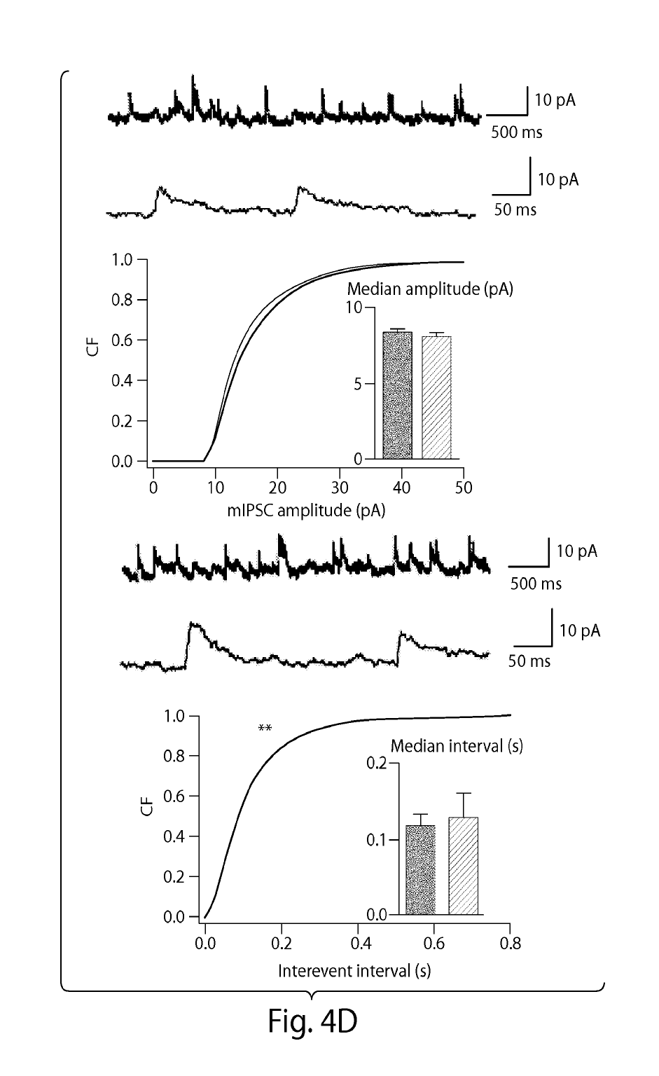

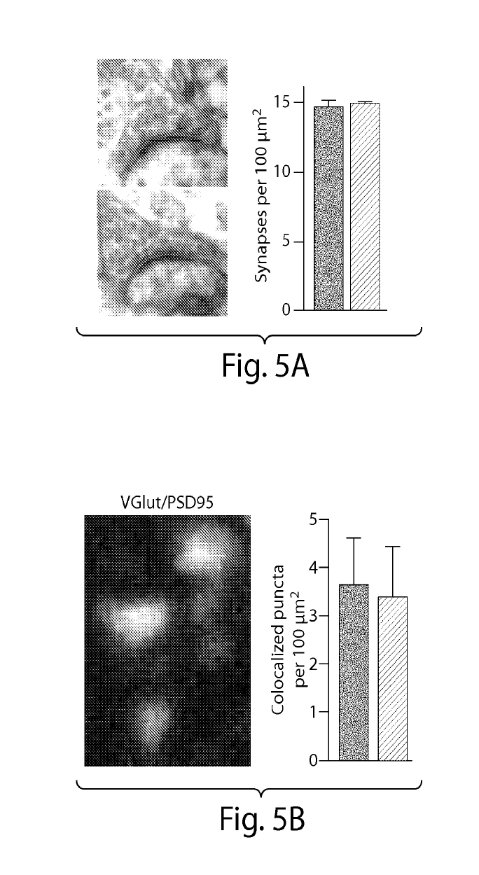

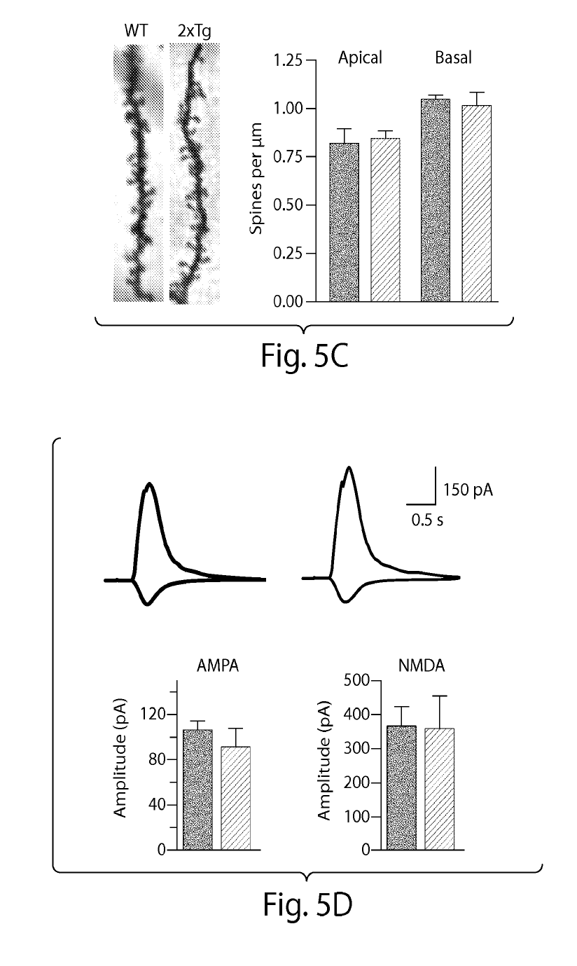

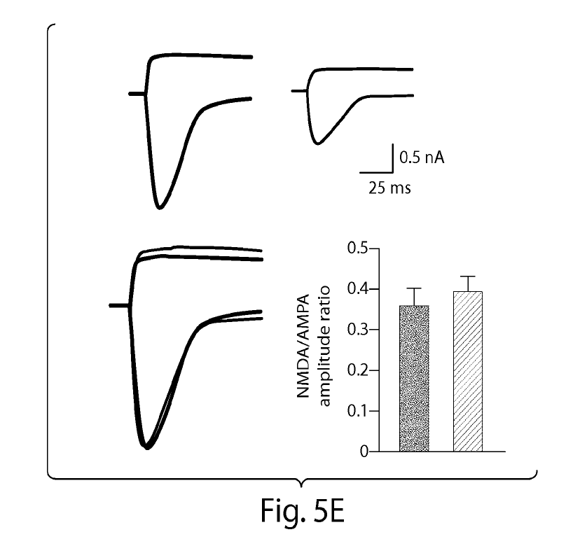

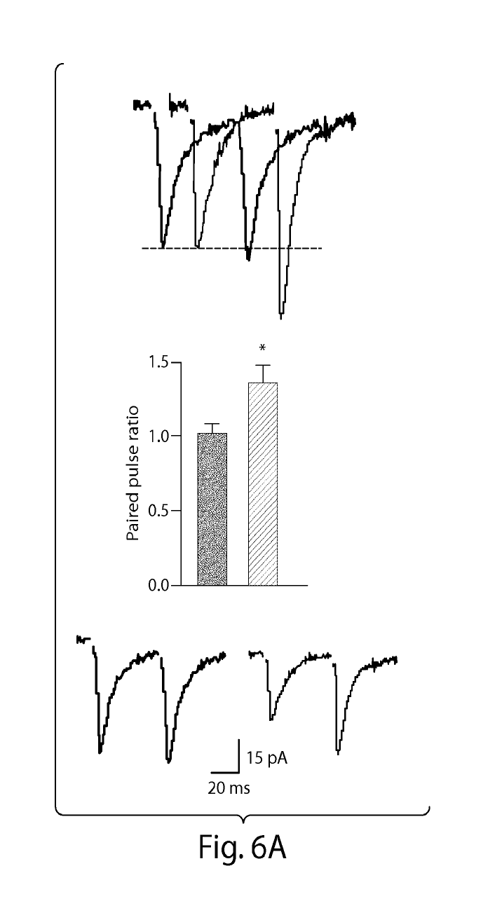

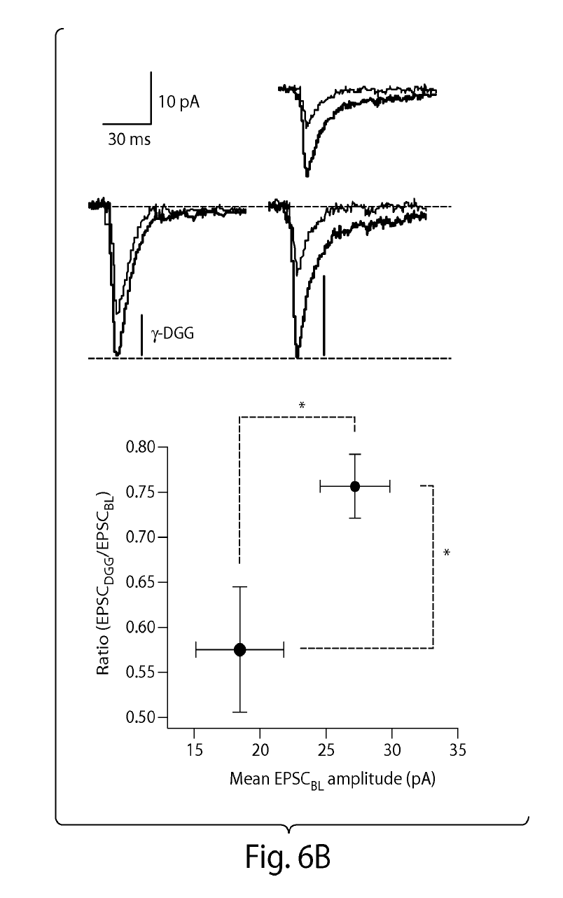

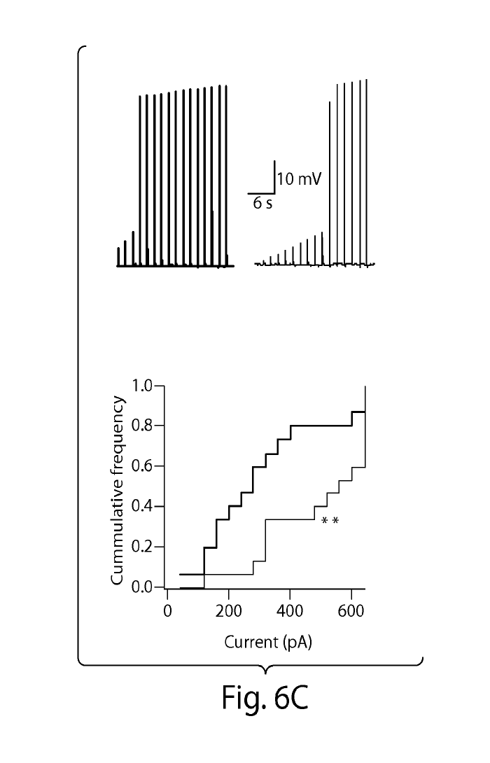

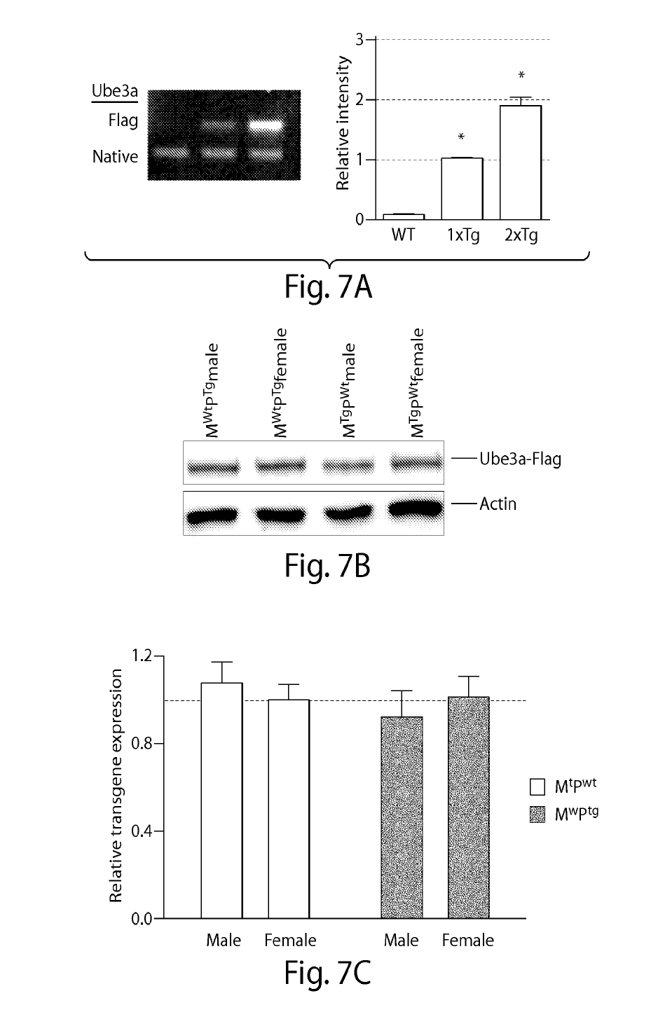

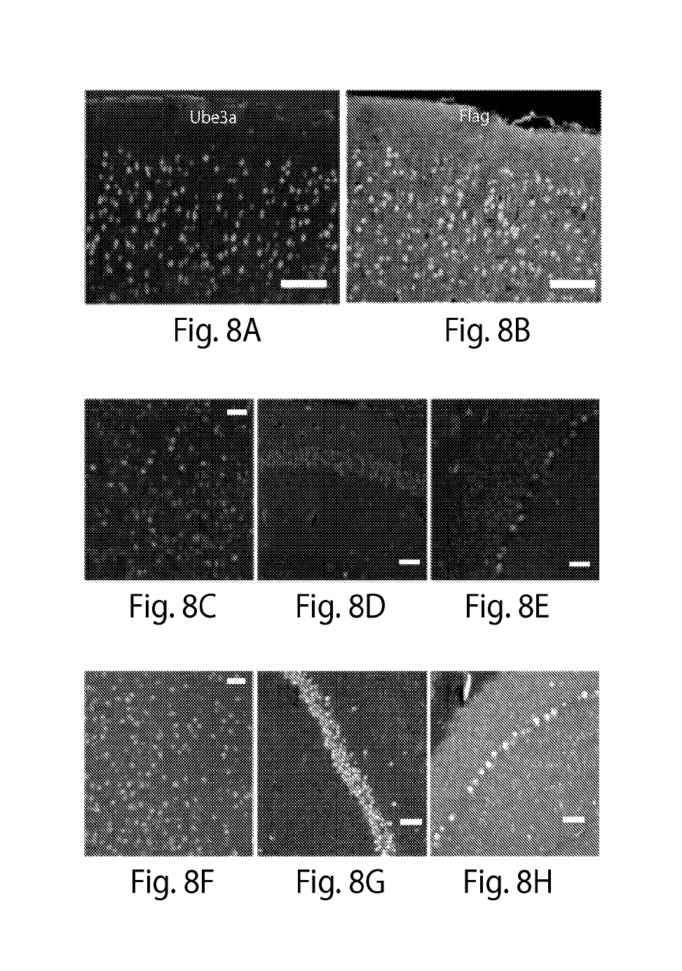

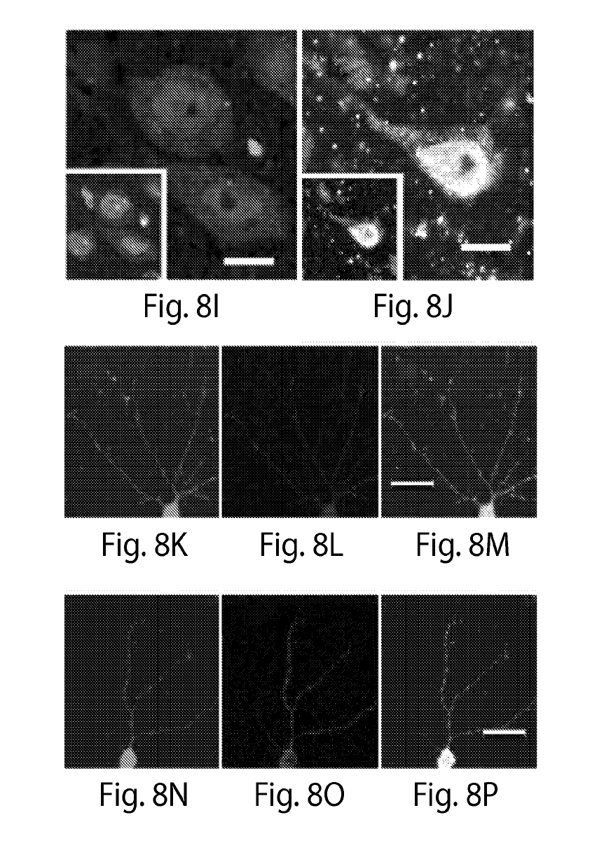

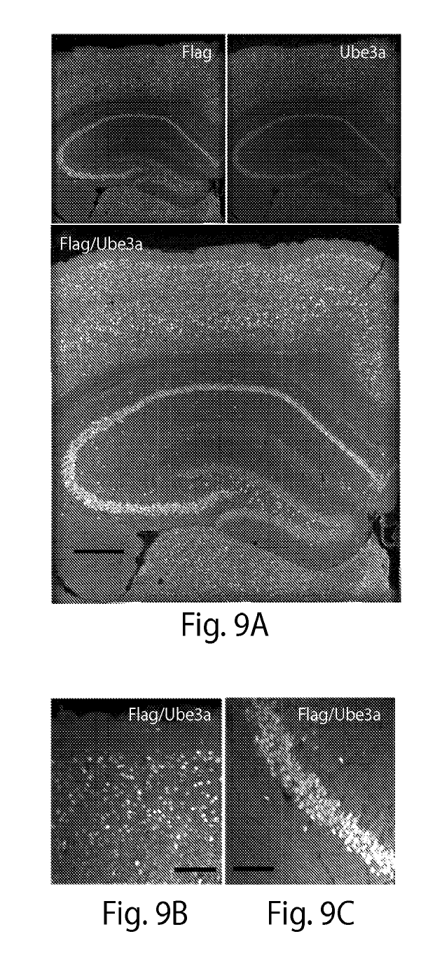

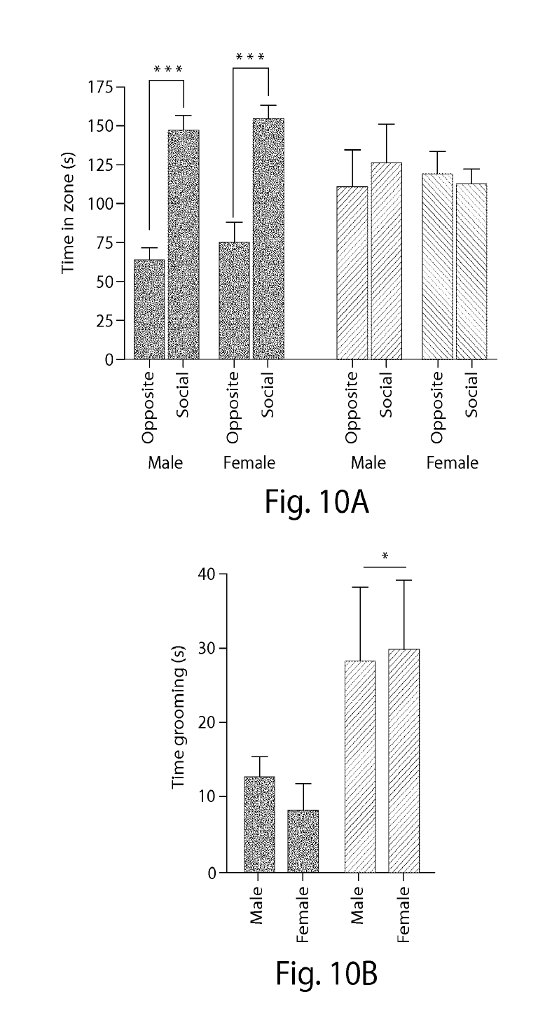

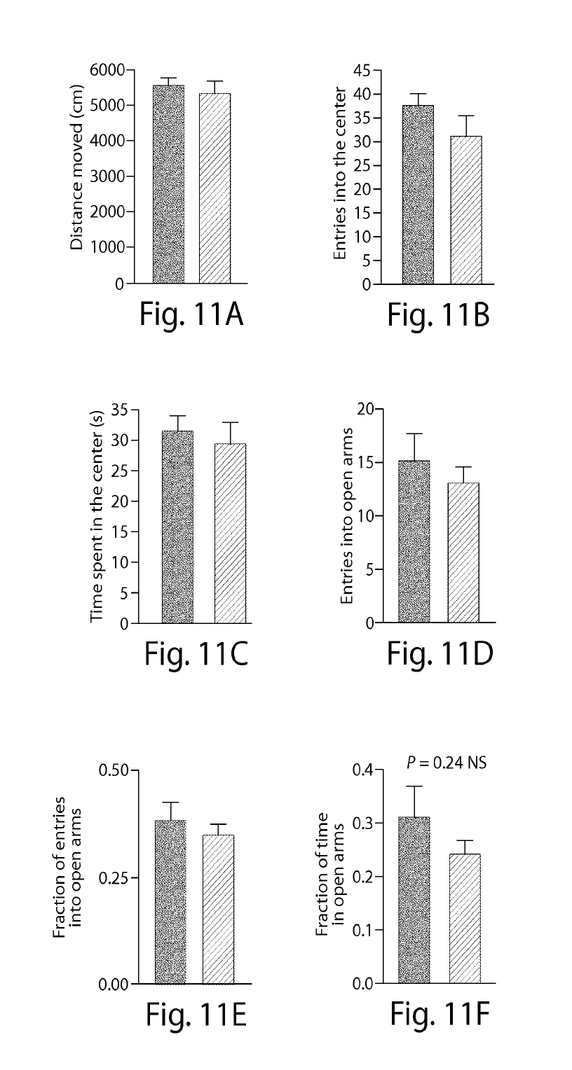

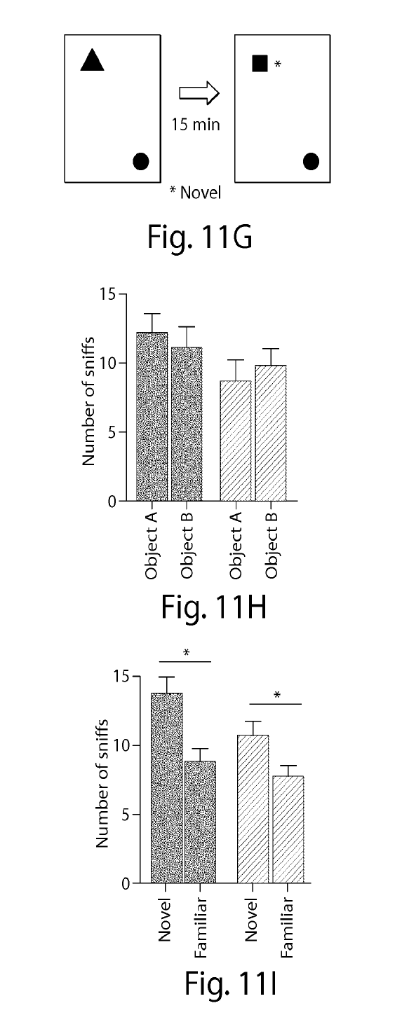

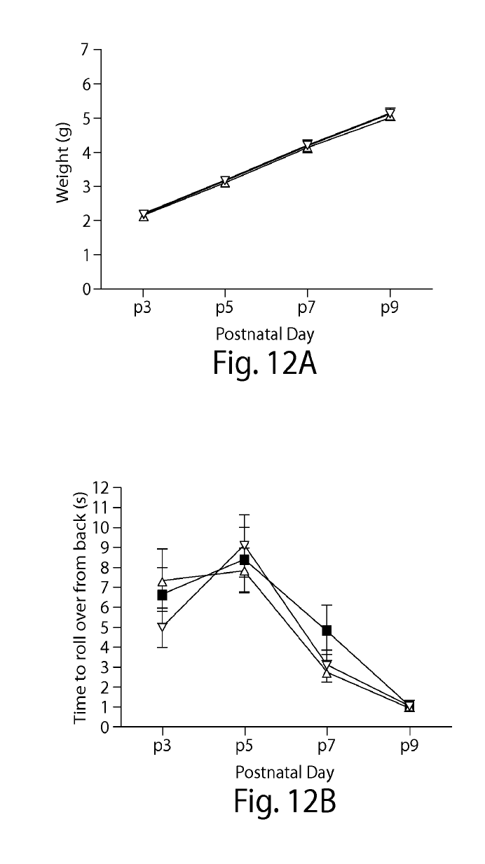

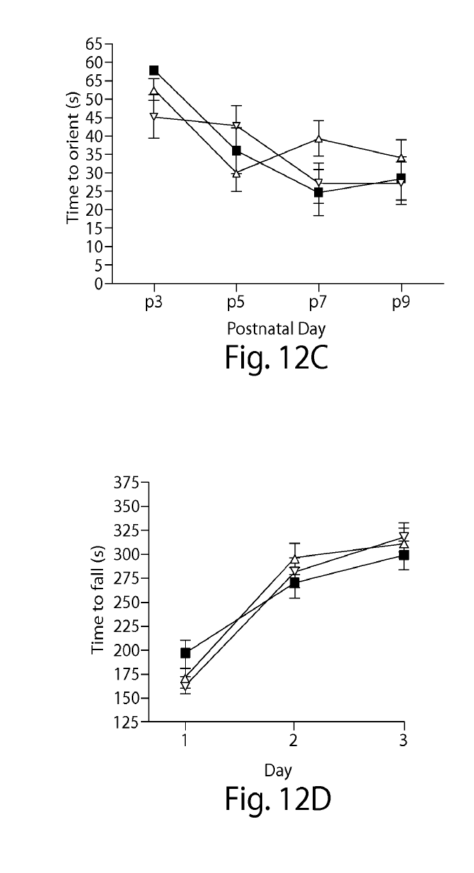

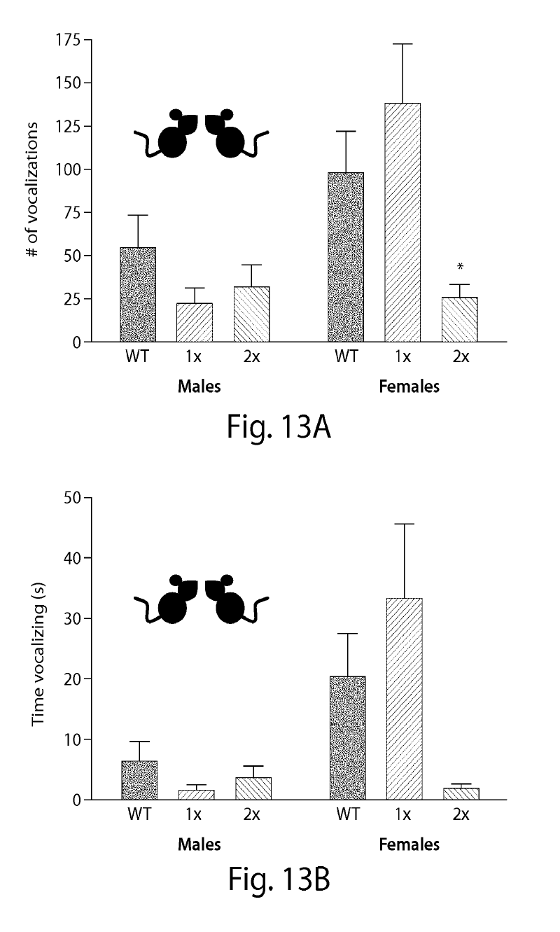

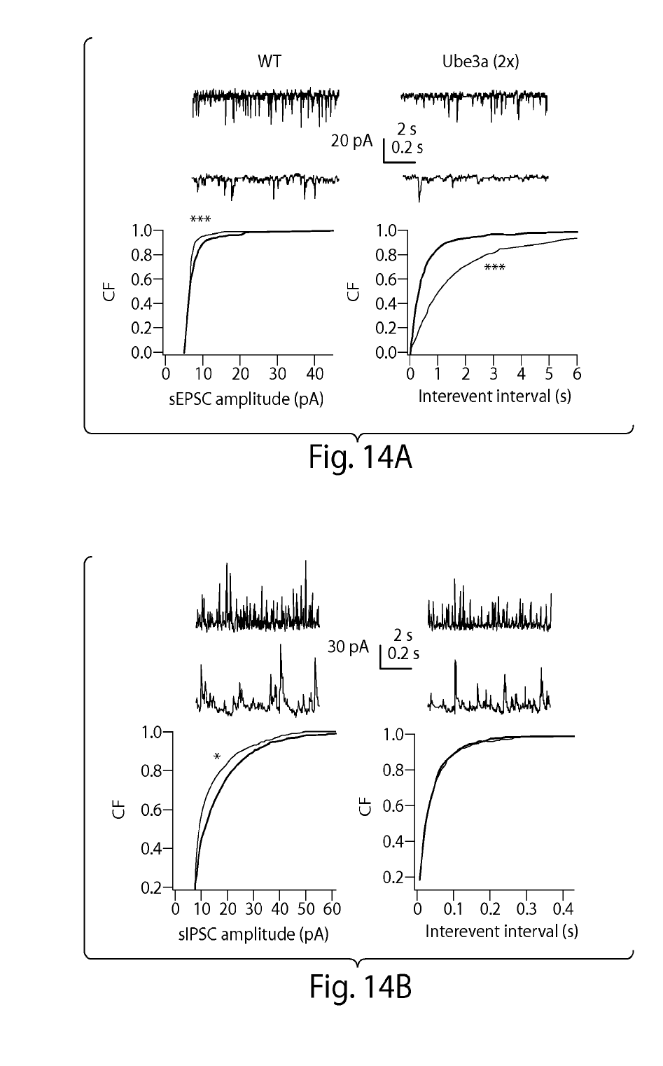

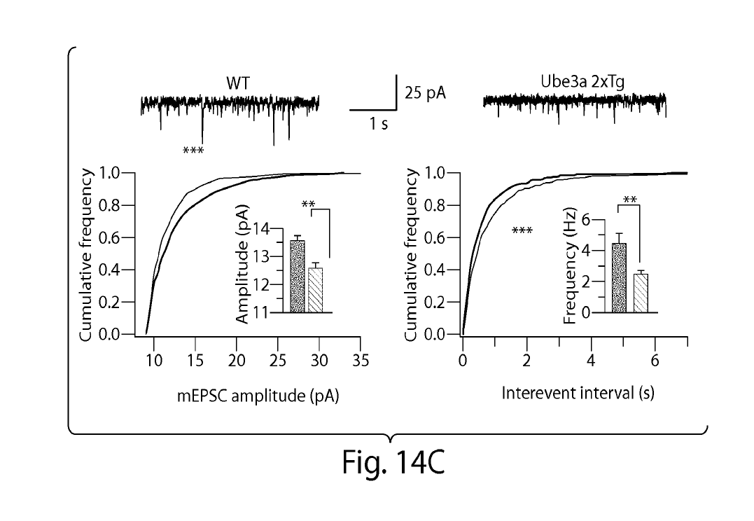

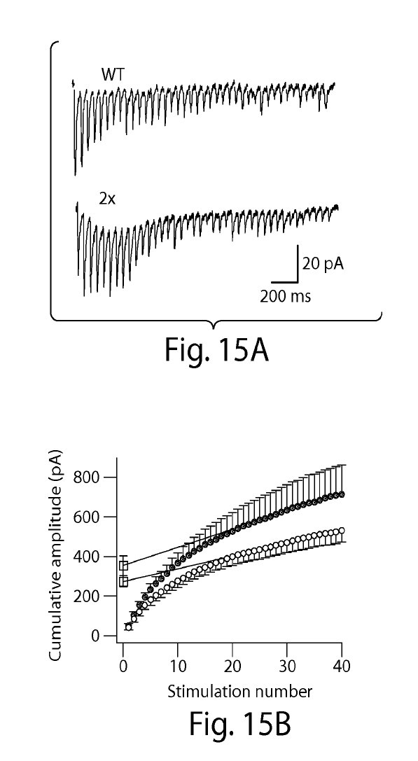

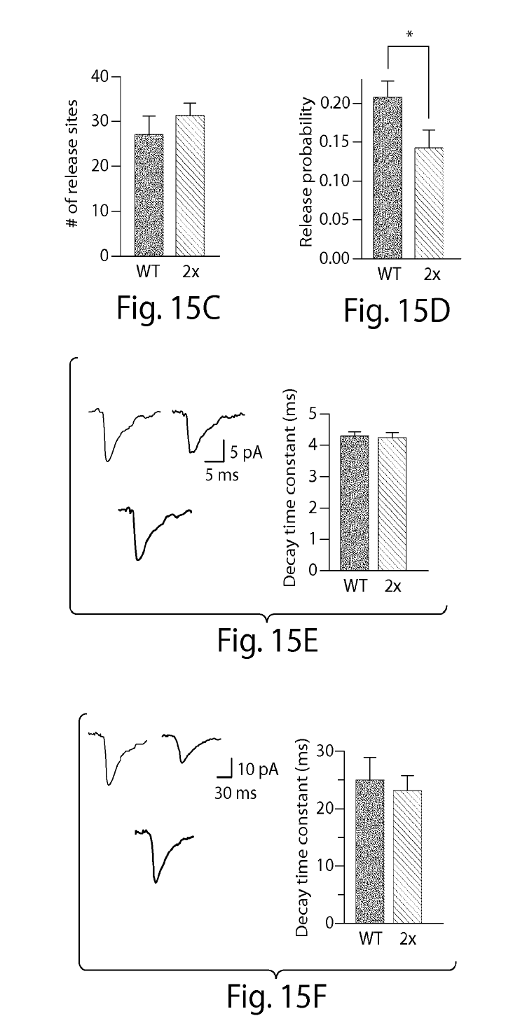

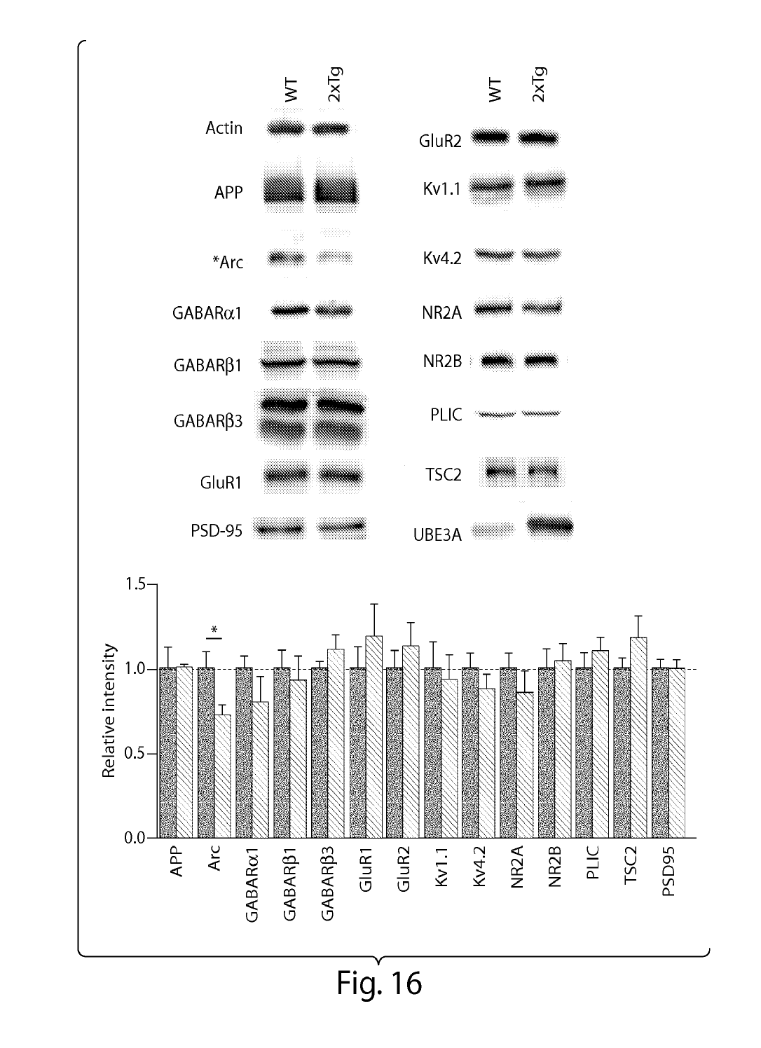

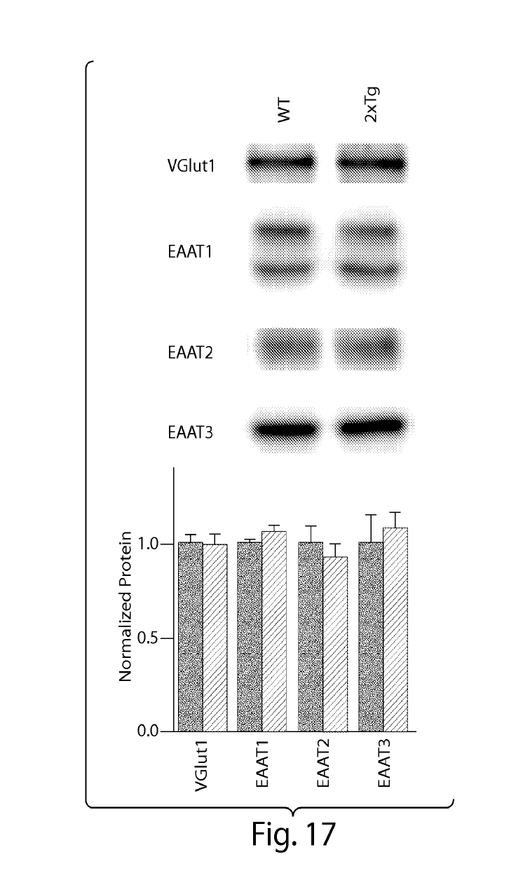

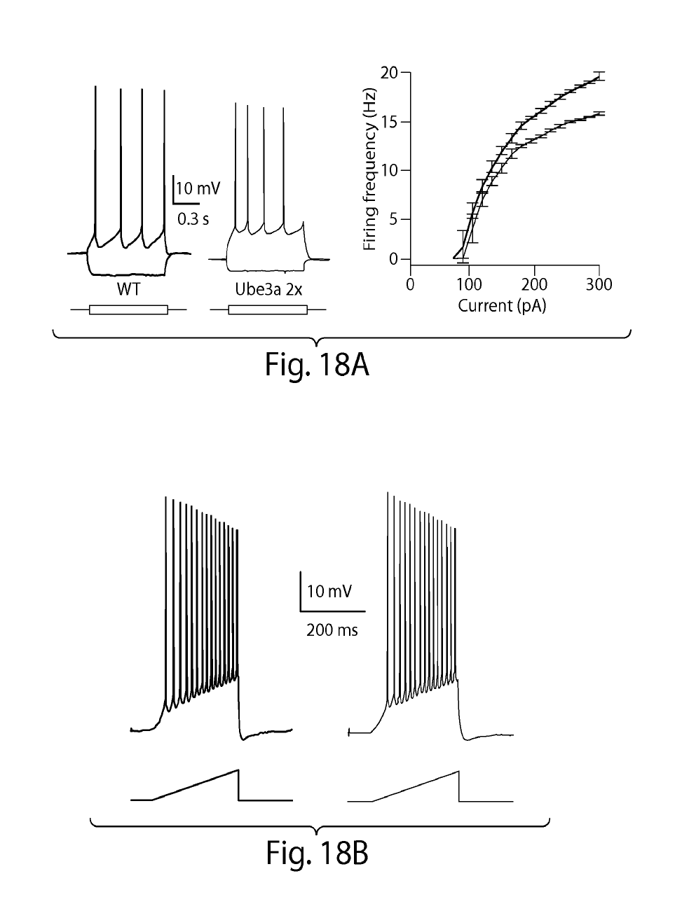

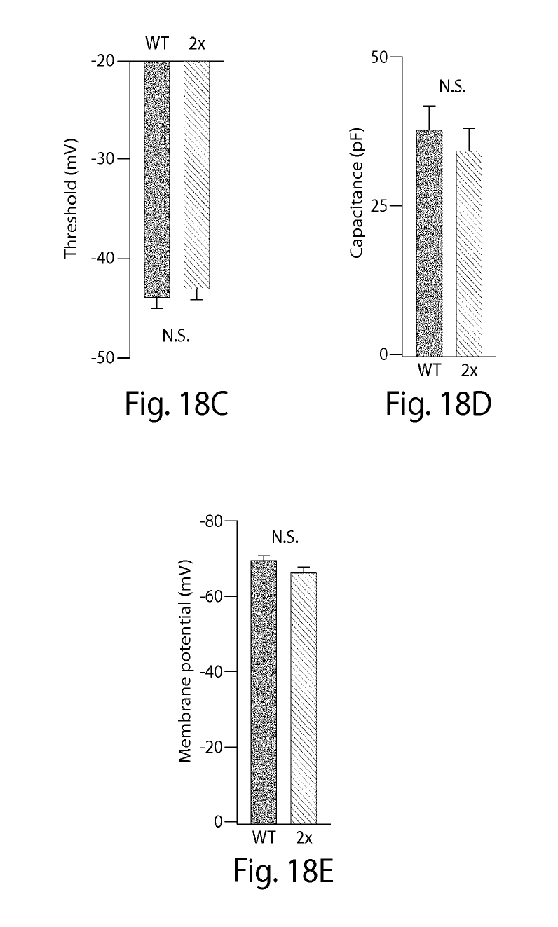

The hypothesis was developed that Ube3a mediates the autism-related behavioral phenotypes associated with dup15 and idic15 because of its known roles in neurologic function and because Ube3a is the only gene in the region known to be expressed exclusively from the maternal chromosome (FIGS. 1 A and B). Using BAC recombineering techniques (21) a 162 kb segment of mouse chromosome 7, containing the entire 78 kb exon-intron coding sequence of Ube3a as well as its 63 kb 5′ and 21 kb 3′ sequences, was inserted into FVB embryos to generate transgenic mice, which were subsequently bred to produce single (1×) and double (2×) copy transgenic animals (FIGS. 1 A-F). A 3×FLAG tag followed by two stop codons was inserted in frame after exon 12 to produce the full-length FLAG-tagged transgenic protein (FIG. 1 A). Two independent transgenic founder lines with independent insertion sites were compared to control for any potential insertion-site effects (Ube3a, founder lines 1 and 2); line 1 is used throughout except where otherwise noted.

Ube3a 1× and 2× transgenic mice were identified by semi-quantitative PCR (FIG. 7A) and confirmed by western blot to show the expected 2- and 3-fold increase of brain Ube3a protein (FIG. 1C). The endogenous Ube3a gene is expressed only from the maternal chromosome in neurons, but the Ube3a transgene expresses independent of parent-of-origin due to the lack of the antisense transcript initiation site underlying imprinting (22) (FIGS. 7B and C). The transgene also expresses independent of sex (FIG. 7C). Immunofluorescence staining for FLAG in transgenic animals recapitulated the native Ube3a staining patterns seen in wild-type, and matched previously reported Ube3a expression patterns (17, 23) (FIG. 8). Further, dual immunofluorescence staining to FLAG and Ube3a showed complete overlap in cortex, hippocampus, and thalamus, indicating the transgenic protein expresses in all cells expressing native Ube3a (FIGS. 1F and 9). The Ube3a transgene generates a functional ubiquitin ligase. Recombinant Arc, one of many known Ube3a targets (19), is degraded in-vitro by FLAG antibody immunoprecipitated transgenic Ube3a (mean±SEM Wt: 1.26±0.04 vs. Tg: 0.58±0.16; P=0.01 by t test; n=3 independent in vitro assays). Further, Ube3a (2×) transgene decreased endogenous Arc in barrel cortex (mean±SEM Wt: 1.00±0.11 vs. 2×: 0.72±0.07; P=0.03 by t test; n=10-12).

### Increasing Ube3a Gene Dosage Impairs Social Behavior

Impaired social interaction is a hallmark autism trait and was assessed in a three-chamber social interaction test (24) where mice choose between a social chamber containing a caged, sex- and age-matched wild-type stranger mouse or the opposite chamber with an empty cage (see diagrams, FIG. 2). Wild-type mice displayed a normal social preference (FIG. 2 A-C), spending more time on the side of the apparatus containing the probe mouse (FIG. 2B) and more time interacting with the probe mouse (FIG. 2C). Ube3a (2×) transgenic mice failed to show a social preference in either measure, while Ube3a (1×) transgenic mice showed an intermediate phenotype, failing to show a preference for the social zone but showing a significant social interaction preference (FIGS. 2 B and C). Comparisons across genotype are reportedly not valid and therefore were not performed (24). Plotting of the middle zone time shows the preference for the outer two zones containing the novel objects (cages) in all strains (FIG. 2B). We then repeated a different version of the three chambered social interaction test on a new cohort with acclimation of the adult mice to both the box and the cages (FIGS. 2 D-F) (25). In this paradigm, wild-type and Ube3a (1×) transgenic mice displayed a more robust preference for the side containing the novel mouse (FIG. 2E). Ube3a (2×) transgenic mice again showed no preference for the side containing the mouse, whether assessed as time in the social zone (FIG. 2E) or in close proximity to the probe mouse (FIG. 2F). This experimental design permitted us to add a novel object rather than the probe mouse with the cage and revealed that Ube3a (2×) transgenic mice, like controls, display a normal preference for this immobile novel object (FIG. 2G). Preserved novel object preference in the Ube3a (2×) transgenic mouse was also demonstrated in the setting of an object memory test (FIG. 11). Two independent Ube3a (2×) transgenic founders (lines 1 and 2) displayed the same deficit in preference for the novel mouse, eliminating insertion-site effects (FIG. 2H). The Ube3a (2×) transgene also abolished the social preference equally in both males and females (FIG. 10A). The results establish that increases of Ube3a gene dosage impair a mouse's normal preference to seek interactions with another novel mouse; a potential mouse correlate of social behavior.

Ube3a (2×) transgenic mice, like controls, displayed normal object exploration and object memory, open field exploration, elevated plus maze behavior, motor function, and developmental milestones eliminating other behavioral deficit confounds (FIGS. 11 and 12, and Table 1). The results indicate a 3-fold increase of Ube3a protein, typical of idic15, impairs mouse-mouse interactions, a potential correlate of the social behavior deficits found in human autism; while a 2-fold increase of Ube3a, typical of dup15, causes more limited deficits in this behavioral paradigm.

### Increasing Ube3a Gene Dosage Impairs Communication and Increases a Repetitive Behavior

Defective communication is the second diagnostic criteria of autism, manifesting as reduced speech in patients. Thus, we assessed whether excess Ube3a impairs communicative ultrasonic vocalizations in mice (FIGS. 3 A-F). We measured two types of social-behavior relevant vocalizations in adult rodents: vocalizations generated by same-sex pairs encountering each other for the first time; and vocalizations generated by sexually experienced males exposed to female urine.

Social stimulus-induced vocalizations were assessed when two age-, sex-, and genotype-matched, non-littermate stranger mice were paired. Wild-type mice emitted vocalizations that were markedly reduced by the 2×, but not the 1×, Ube3a transgene dosage (FIG. 3A). The effects of Ube3a were most pronounced in females, as female pairs vocalized with a much greater frequency than males (FIG. 13). As previously reported (26), female urine elicited a diverse repertoire of vocalizations in sexually-experienced, wild-type males (FIGS. 3 B-D). In this test, however, the number of vocalizations and the time spent vocalizing were strongly reduced in both 1× and 2×Ube3a transgenic mice (FIG. 3B). Despite the large decrease of vocalization quantity, other vocalization features were unaffected: average peak frequency (67±1, 67±2, 69±2 KHz, respectively) and call type distribution (FIG. 3D). To control for a potential confounding deficit in olfaction, mice were tested in an olfactory habituation/dishabituation test, which confirmed the ability of transgenic animals to respond normally to novel scents presented on a cotton swab (FIG. 3E). Pup vocalizations during a five minute separation from their mother, an aversive stimulus (27), were unaffected by the increased Ube3a gene dosage, indicating a preserved function of the vocalization system (FIG. 3F). The results establish that the ultrasonic vocalizations generated in a social context are impaired by increases of Ube3a gene dosage. While not currently relevant to the diagnosis of autism, the results uncover a robust defect in the male vocalization response to sexually motivating chemicals (possibly pheromones) in female urine in both the dup15 and idic15 models.

The third core autism trait is repetitive, stereotyped behaviors such as body rocking, hand flapping, or self-injurious behavior. Repetitive self-grooming has been assessed in mice as a correlate of repetitive behavior (24, 28, 29). Self-grooming was increased 3-fold in Ube3a (2×) transgenic mice, but unaffected in Ube3a (1×) transgenic mice, compared to wild-types (FIG. 3G). Both Ube3a transgene founder lines 1 and 2 produced the same 3-fold increase of self-grooming relative to control littermates (FIG. 3H). Increased self-grooming was also observed in both male and female Ube3a (2×) transgenic mice (FIG. 10B). In summary, a 3-fold excess of Ube3a, as found in idic15, generates correlates of the three core autism-related behavioral traits in mice, while a 2-fold excess of Ube3a generates deficits in only a limited subset of the behaviors (male sexual vocalizations).

### Increasing Ube3a Gene Dosage Impairs Glutamatergic Synaptic Transmission

Ube3a is present in the cytoplasm and is concentrated in the nucleus and at distinct postsynaptic density protein (PSD-95) positive puncta distributed along the dendrite (FIGS. 8 I-P). We hypothesized that increasing Ube3a gene dosage might alter evoked excitatory and/or inhibitory postsynaptic currents (EPSCs and IPSCs) in cortical pyramidal neurons. We measured somatosensory whisker barrel cortex layer 2/3 pyramidal neurons, because the neurons are highly active (Ube3a is an activity-induced gene (19)) strongly express Ube3a (FIGS. 8 A and B) (19, 23), and, importantly, they have been previously studied in other autism spectrum disorder muse models. Barrel cortex layer 2/3 pyramidal neurons displayed increased or decreased GABAergic transmission in the autism-associated neuroligin 3 (NLGN3) point mutation mouse (30) or the Rett syndrome (MeCP2) mouse (31), respectively, without affecting glutamatergic synaptic transmission.

Synaptic currents were measured by patch-clamp recording barrel cortex pyramidal neurons in acute brain slices. Ube3a (2×) transgenic mice displayed strong suppression of evoked excitatory post synaptic currents (EPSCs) (FIG. 4A). By contrast, evoked inhibitory post synaptic currents (IPSCs) were not significantly reduced (FIG. 4B). We measured spontaneous IPSCs and EPSCs, and found reduced spontaneous EPSC amplitude and frequency (FIG. 14A). Spontaneous IPSCs had reduced amplitudes, but unaltered frequencies (FIG. 14B). Thus, increasing Ube3a gene dosage as found in idic15 strongly reduced glutamatergic synaptic transmission with more limited effects on GABAergic transmission.

To more directly examine how increased Ube3a gene dosage down-regulates glutamate synapse function, miniature EPSCs and IPSCs (mEPSC and mIPSC) were measured with action potentials inhibited. Ube3a (2×) strongly suppressed mEPSC amplitude and frequency (FIG. 4C). By contrast, and unlike the autism-associated NLGN3 point mutant and Rett syndrome mouse models (30, 31), excess Ube3a failed to significantly alter miniature IPSC frequency or amplitude (FIG. 4D). To be sure that the reduced mEPSC frequency was not due to reduced mEPSC amplitude (falling below detection threshold), we clamped at −80 mV to increase amplitude and again found a reduced mEPSCs amplitude and frequency (FIG. 14C). Decreases of mEPSC amplitude and frequency suggested increased Ube3a gene dosage regulates glutamate synaptic transmission at both pre- and post-synaptic sites.

### Increasing Ube3a Reduces Presynaptic Glutamate Release Via Two Mechanisms

Synaptic glutamate release (R) is governed by the equation R=Npq, where N is number of release sites, p is probability of release, and q is quantal size. We suspected a change in synapse number might contribute to the decreased glutamate release by analogy to the maternal Ube3a knockout mice that display fewer dendritic spines (12, 17). Therefore we evaluated for changes in glutamate synapse number in layer 2/3 barrel cortex using three independent measures: counting asymmetric synapses using electron microscopy (FIG. 5A); counting the number of co-localized vglut1-PSD95 puncta in thin (5 μm) sections by dual immunofluorescence staining (FIG. 5B); and counting dendritic spines in Golgi-stained sections (FIG. 5C). By all measures, synapse number was unaltered by the increased Ube3a (2×) gene dosage. Furthermore, the number of release sites, assessed electrophysiologically (see below), was unaltered (FIG. 15). Thus, changes in N fail to explain the reduced mEPSC frequency.

Presynaptic release probability (p) was measured directly using a repeated minimum stimulation protocol (FIGS. 15A-D, see Supplementary Experimental Procedures). Release probability was significantly reduced in Ube3a (2×) transgenic mice (FIG. 15D). Paired-pulse ratio, increased at low-release-probability synapses, was increased in Ube3a (2×) transgenic mice (FIG. 6A). Both measures suggest that increasing Ube3a gene dosage decreases the release probability (p) of cortical glutamate synapses to reduce mEPSC frequency and thereby lower the efficacy of glutamatergic synaptic transmission.

Quantal size (q) can be altered by pre- or post-synaptic changes, and reduced mEPSC amplitude often results from decreased post-synaptic AMPA receptor density. To directly measure the post-synaptic response, we applied small puffs of glutamate to slices and recorded post-synaptic AMPA and NMDA currents. Glutamate iontophoresis induced AMPA and NMDA receptor currents were similar in amplitude across genotypes (FIG. 5D). Further, evoked AMPA and NMDA currents displayed a similar AMPA/NMDA ratio (FIG. 5E) and AMPA and NMDA current kinetic across genotypes (FIGS. 15E and F). We were unable to detect a change in the total quantity of glutamate receptor subunits and other synaptic proteins when measured by western blot of micro-dissected barrel cortex (FIG. 16). The results suggest increases of Ube3a gene dosage do not reduce glutamatergic synaptic transmission and quantal size (q) by decreasing postsynaptic glutamate receptor function.

The reduced mEPSC amplitude with preserved post-synaptic glutamate receptor function suggested a possible decrease of synaptic glutamate concentration. To test this hypothesis, we applied a weak glutamate receptor antagonist γ-DGG (γ-D-glutamylglycine) to assess for glutamate concentration-dependent decreases of synaptic transmission. A greater percent inhibition by γ-DGG indicates a lower synaptic glutamate concentration (see (32)). γDGG did indeed reduce EPSC amplitude to a greater extent in Ube3a (2×) transgenic than in wild-type neurons (FIG. 6B). The decreased synaptic glutamate could not be explained by decreases of synaptic vesicle diameter (measured by electron microscopy: Wt 37.40±0.22 nm, Ube3a (2×) 37.37±0.19 nm) or glutamate transporter proteins (VGlut1 or EAAT1-3, FIG. 17).

### Ube3a Reduces Postsynaptic Excitability to Phasic Synapse-Like Stimuli

Effective glutamatergic synaptic transmission also depends on the coupling of EPSCs to postsynaptic action potential firing. EPSC-spike (ES) coupling was assessed with short (5 ms) EPSC-like current injections directly into the patch-clamped neuron, bypassing the defects already shown to be present at synaptic inputs. This measure assesses the intrinsic excitability of the neuron, compared to EPSC measures which assess synaptic inputs from surrounding neurons. Ube3a (2×) transgenic mice displayed impaired ES coupling (FIG. 6C). By contrast, action potential threshold, capacitance, and resting membrane potential were unaltered, while there was some evidence of a defect at peak firing rates (FIG. 18). The results indicate that in addition to the strong impairment of glutamatergic synaptic transmission, the ability of these phasic excitatory synaptic inputs to activate action potentials will also be severely impaired.

In summary, excess Ube3a acts at multiple, but specific sites within the pre- and post-synaptic compartments to impair glutamatergic synaptic transmission; decreasing presynaptic release probability, synaptic glutamate concentration, and postsynaptic ES coupling. By contrast, glutamate synapse densities were unaltered and GABAergic synaptic transmission showed only minor changes.

### Discussion

To understand the cellular, molecular, and circuit abnormalities behind the behavioral defects of autism, the ideal mouse model would meet three criteria: 1) based on a well-characterized and relatively common human risk factor; 2) phenocopy the human behavioral disorder; and 3) lack confounding co-morbidities. To date, several mouse models-based on rare single gene point mutations (e.g., NLGN3 (30, 33)), syndromic disorders with partial autism penetrance (e.g., Tuberous Sclerosis, Fragile X, Rett Syndrome, see (34, 35)), or mouse social behavior screens (e.g., BTBR mouse (36))—phenocopy a subset of behavioral components of the disorder. These models have led to great progress in understanding the genetic control of social behavior and in some cases in identifying the underlying mechanisms of these specific neurodevelopmental disorders. The Idic15 mouse model with extra copies of the ube3a gene is based on one of the most common known risk factors for autism (1-3% of cases), shows strong penetrance of the three core autism-related behavior traits, and has not been found to display other major co-morbidities. Therefore, further studies of the mechanism whereby Ube3a causes behavioral and circuit abnormalities will provide new insights into human idic15 autism. More importantly, comparison of the circuit defects in this idic15 mouse model with other autism models may yield insights into the elusive pathophysiological mechanisms of the disorder.

The 15q11-13 duplicated region contains at least 30 characterized genes, several previously proposed to potentially underlie the autism phenotype. ATP10A was of interest because early studies suggested that it, like Ube3a, might express exclusively from the maternal chromosome (37, 38). However, this has since been refuted by several other groups (39, 40). Other genes within the duplicated genomic region, such as GABAA receptor subunits β3, α5 and γ3 and cytoplasmic FMRP-interacting protein 1 (CYFIP1), have been proposed to mediate the autism risk (8, 41), but none are imprinted in a way that readily explains the selective association of autism with maternally-inherited duplications. Although we cannot rule out a contribution from these other genes, our results indicate Ube3a alone is sufficient to replicate the core autism-related traits in mice.

Furthermore we performed a direct comparison of mice with Ube3a gene duplication and triplication and found gene-dosage effects on autism-related trait penetrance as previously observed in humans. A 3-fold increase of brain Ube3a protein, as predicted for isodicentric extranumerary chromosome (idic15), associated with full autism penetrance, reconstituted surrogates of all three core autism-related behavioral traits. By contrast, a 2-fold increase of brain Ube3a protein, as predicted for inverted duplication of 15q11-13 (dup15), associated with weaker autism penetrance, generated only a subset of the behavioral defects (most strongly, reduced male vocalizations to female urine).

These dose-dependent effects of Ube3a may explain the lack of autism traits in a recently developed mouse model aimed at reconstituting the maternally-inherited autism disorder associated with dup15 (40). The authors used an elegant chromosome-engineering technique to replicate dup15 in mice, and demonstrated the expected maternal and paternal-specific gene expression patterns of the imprinted chromosomal region. Yet, despite successful reconstitution of the typical maternal-selective expression of Ube3a in brain and a doubling of brain Ube3a mRNA levels, autism-related behavioral traits were not observed with maternally-inherited 15q11-13 duplications. Our finding that a tripling of Ube3a protein dosage, typical of idic15, is necessary to reconstitute the full set of autism-like traits in mice may explain the lack of phenotype in the this mouse model. Two extra copies of maternally inherited Ube3a, as in idic15, cannot be generated using this chromosome engineering technique preventing a direct comparison of the gene dosages that we achieved using bacterial artificial chromosome transgenic techniques.

With no changes in resting membrane potential, action potential threshold, and input resistance, what mechanisms might explain the strong suppression of ES coupling? There are two possibilities. First, increases of a rapidly inactivating, low threshold K⁺ channel (e.g., Kv4) would not contribute to resting membrane potential or input resistance and would be inactivated during the slow ramped current used to assess action potential threshold. Second, increases of a calcium-activated K+ channel (e.g., SK) would also not contribute to resting membrane potential or input resistance and could be activated by the calcium influx through a low threshold T-type Ca²⁺ channel. This calcium channel would also be inactivated by the slow ramp current. Increases of SK channels might also help explain the suppression of peak firing rate as intracellular calcium will accumulate with repeated action potential firing (42).

The glutamatergic synaptic defects we report in these mice with increased Ube3a gene dosage are not those predicted from simply inverting the effects previously observed in the Angelman syndrome mouse model with maternal Ube3a knockout. For example, Yashiro et al. (17) reported reduced mEPSC frequency in maternal Ube3a knockouts, an effect we also report with increased Ube3a gene dosage. Similarly, Greer et al. (19) reported reduced glutamatergic synaptic transmission and reduced AMPA mEPSC amplitude in maternal Ube3a knockout mice that they attributed to a lack of Ube3a-promoted Arc degradation leading to fewer AMPA receptors at the synapse (43). Yet, while we confirmed that increasing Ube3a gene dosage partially reduces total Arc as predicted, AMPA currents were not increased as predicted if changes this molecule mediated the effect. These results indicate too little or too much Ube3a can depress glutamatergic synaptic transmission. Maternal Ube3a knockout has also been reported to reduce dendritic spine density (12, 17), yet increasing Ube3a gene dosage did not increase (or decrease) dendritic spine density or glutamate synapse density. While both high and low levels of Ube3a cause human neurologic diseases, our findings suggest the molecular and circuit mechanisms leading to Angelman syndrome and idic15 autism may be quite different.

Ube3a is an E3 ubiquitin ligase, a class of proteins that provide substrate specificity to the ubiquitin protein degradation system. Many tens of targets of Ube3a have been identified in cell culture systems (44, 45), Drosophila (46, 47), and recently in mouse brain (19, 20). Our initial screen of some of these potential Ube3a targets so far has only revealed a 30-40% decrease in Arc. The functional glutamate synapse defects (presynaptic release probability, glutamate loading of vesicles, and ES coupling) produced by excess Ube3a are distinct from those predicted to result from reduced Arc and instead suggest several distinct ubiquitination targets may exist within pre- and post-synaptic compartments that remain to be identified. Ube3a also acts as a steroid hormone transcriptional co-activator independent of its E3 ligase activity (48, 49) and its strong nuclear localization, potentially important effects in the regulation of gene expression should also be considered.

Reconstituting all three core autism traits in the mouse using a single gene within this large (5 Mb) and common (1-3%) human autism-associated genomic copy number variation establishes the feasibility of investigating the cellular and circuit basis of human idiopathic autism disorders due to copy number variations. In the future, comparing the circuit defects across multiple genetically-determined autism mouse models may reveal fundamental core mechanisms responsible for this disorder. For example, both Neuroligin 3 mutant and Neurexin 1α deficient mice show increased GABAergic synaptic transmission predicted to decrease cortical circuit excitability (29, 30). By contrast, increasing Ube3a gene dosage reduces circuit excitability by reducing glutamatergic synaptic transmission. By reconciling the differences and commonalties between mouse models of the human genetic autisms, various autism subtypes may soon be defined that respond to distinct treatments and translate into a variety of therapies useful for the larger autism patient population.

### Materials and Methods

### Generation of Mice

Using BAC recombineering techniques we inserted a 162 kb segment of mouse chromosome 7, containing the entire 78 kb exon-intron coding sequence of Ube3a as well as its 63 kb 5′ and 21 kb 3′ sequences, into FVB embryos to generate transgenic mice (FIG. 7). A 3×FLAG tag followed by two stop codons was inserted in-frame after exon 12 to generate two independent founder mice. Transgenic founder offspring were then crossed to produce Ube3a 1× or 2× transgenic mice. Genotyping was accomplished by semi-quantitative PCR of sequences flanking the FLAG-tagged Ube3a gene with the native gene as an internal control. Primers and detailed protocols are available in the Extended Experimental Procedures.

### Ube3a Protein Over-Expression and Function

Ube3a expression was confirmed by western blot of cortical lysates using both anti-FLAG M2 antibody (Sigma) and anti-Ube3a (BD Biosciences). The ubiquitin ligase activity of Ube3a was assayed by an in vitro target protein degradation assay. Ube3a was immunoprecipitated using anti-FLAG M2 antibodies and protein G magnetic beads (NEB) and eluted in non-denaturing conditions using a 3× FLAG peptide (Sigma). 1 μg of recombinant ARC (BD Biosciences) was added to 10 μl of immunoprecipitated Ube3a in the presence of 50 ng E1 and 100 ng UbcH7 enzymes and 4 μg HA-ubiquitin (Boston Bioproducts) as in (19). Western blots were probed with anti-ARC (Santa Cruz) and quantified using ImageJ (NIH). Detailed protocols are available in the Extended Experimental Procedures.

### Three Chamber Social Testing

Separate cohorts of mice were tested in the three chamber social test as either juveniles (3-4 week) or adults (8-12 weeks) following previously published protocols (25)). For the juvenile test, following a ten minute acclimation period in an empty chamber, a stranger wild-type mouse was placed in a small enclosure in one of the outer chambers, and an empty enclosure was placed in the opposite side. The round wire enclosure (a pencil holder, Office Depot) allowed visual, olfactory and tactile interaction. The test session lasted 10 minutes. For the adult test, the enclosures were present during the acclimation, and sessions lasted 5 minutes. Therefore, in the juvenile test, the comparison was between a novel mouse and a novel object (the enclosure) while the adult test compared a novel mouse with a familiar container. To control for novel object preference in the adult, we repeated the test but placed a novel object (a striped plastic cup) into one of the two enclosures a week after the initial test.

### Grooming

Mice were placed in a clean cage in a fume hood in their home room, and were allowed to acclimate for ten minutes. Mice were then video recorded for ten minutes, and the time spent grooming was measured by an experienced observer (as in (29)).

### Vocalizations

For urine-induced vocalizations, male mice were single-housed for several days, and then exposed to brief (5 min) social interactions with both male and female mice for four days before the test. On the 5^{th} day, mice were placed in a small plastic box inside a larger sound-proof container. A cotton swab dipped in freshly-collected urine pooled from at least 10 females from at least 5 different cages was suspended from the top of the smaller box, so that the tip was approximately 5 cm above the floor. An ultrasonic microphone recorded vocalizations and fed data into a computer running Avisoft-Recorder (Avisoft Bioacoustics) which automatically counted the vocalizations over the five minute test period. For social vocalizations, sex-, age- and genotype-matched, non-littermate mice who had never encountered each other before were placed in a small plastic enclosure simultaneously (to avoid resident-intruder aggression) and the number of vocalizations and time spent vocalizing were recorded automatically (Ultravox, Noldus) for five minutes.

Electrophysiology

Evoked postsynaptic currents were recorded in voltage-clamp mode using cesium-based artificial intracellular fluid and regular ACSF. A bipolar platinum/iridium stimulating electrode (CE2C55, FHC Inc., Bowdoin, Me.) was placed at layer 2/3 of the barrel cortex 200 μm away from the recording site. A glass pipette filled with 0.5 mM bicuculline methiodide (BMI) in ACSF that locally inhibited GABAergic transmission was placed above the soma of the cell being recorded. Inhibitory postsynaptic currents (IPSCs) were recorded at a holding potential of +10 mV in the presence of bath 10 μM DNQX and 50 μM APV. Detailed protocols are available in the Extended Experimental Procedures.

Miniature EPSCs (mEPSCs) and miniature IPSCs (mIPSCs) were respectively recorded at −60 mV or −80 mV and +10 mV. Detailed protocols are available in the Extended Experimental Procedures.

Glutamate Iontophoresis:

Pyramidal neurons were voltage-clamped at −70 mV in the presence of 1 μm TTX and 100 μm picrotoxin. Iontophoretically applied glutamate (10 mM sodium glutamate in 10 mM HEPES, pH 7.4) was delivered through glass pipettes (4-6 MΩ when filled with normal internal solution) placed 1-2 μm away from the main apical shaft (˜15-20 μm from cell body). Detailed protocols are available in the Extended Experimental Procedures.

Minimal Stimulation and Estimation of Vesicle Glutamate Content:

The vesicle glutamate content was estimated by the relative inhibition of mean single fiber EPSC amplitude by the fast off-rate, non-NMDA receptor blocker γ-DGG (300 μM). The higher the percentage inhibition by γ-DGG, the lower the concentration of synaptic glutamate. Detailed protocols are available in the Extended Experimental Procedures.

### Statistical Analysis

For behavior analysis, comparisons between two groups used two-tailed unpaired Student's T-Test. Comparisons among multiple groups used one-way ANOVA with Dunnett's post-hoc test comparing each genotype to wild-type; non-significant comparisons are not stated in the manuscript. Comparisons involving multiple independent variables used two-way ANOVAs. Non-normal data (social vocalizations) were tested using the Kruskal-Wallis test followed by Dunn's multiple comparison post-hoc test comparing each genotype to wild-type. For electrophysiological data, two-tailed unpaired Student's t-test was used to compare group means. Kolmogorov-Smirnov test was used to compare cumulative distributions. Mantel-Haenszel Chi-Square test was used to compare the ES coupling data. Unbalanced two-way ANOVA was used to compare group variance. Tukey's honestly significant difference (HSD) test was used to perform post-ANOVA pair-wise comparisons. N=number of cells analyzed. All data is presented as mean±SEM unless otherwise noted. P<0.05 was considered statistically significant.

### Extended Experimental Procedures

To determine whether excess Ube3a gene copies are sufficient to produce the autism behavioral traits, we used BAC recombineering techniques (Zhou et al. 2009), to insert a 162 kb segment of mouse chromosome 7, containing the entire 78 kb exon-intron coding sequence of Ube3a as well as its 63 kb 5′ and 21 kb 3′ sequences, into FVB embryos to generate transgenic mice. Native and flag epitope-tagged transgenic Ube3a displayed matching patterns of expression across multiple brain areas (FIGS. 8 and 9). A FLAG epitope tag was added to the 3′ end of exon 12. To control for site-of-insertion effects, two independent founder lines (1 and 2) were analyzed. The transgene construct lacks the transcription initiation site of the antisense transcript, which in the endogenous gene is responsible for paternal silencing in brain and is located over 500 kb downstream of the BAC beyond the SNP/SNRPN. We confirmed that expression of FLAG-Ube3a is independent of parent-of-origin or sex of the animal (FIG. 7c).

Generation of FLAG-Ube3a Mice:

We generated FLAG-tagged full length Ube3a using a BAC (RP24-178G7) construct following PCR-based methods in combination with the lambda red recombinase system, as previously described (Anderson et al. 2005, Zhou et al., 2009). The BAC DNA was prepared using double acetate precipitation and CsCl₂ gradient purification methods, and then linearized using the restriction enzyme PI-Sce (NEB) and microinjected into FVB embryos.

All procedures were performed in accordance with animal experimental protocols approved by the Beth Israel Deaconess Medical Center Animal Care & Use Committee, an agency accredited by the Association for the Assessment and Accreditation of Laboratory Animal Care, International (AAALAC). Genotyping was performed as previously described (Anderson et al. 2005; Zhou et al. 2009).

### Characterization of Mice

Antibodies:

Ube3a (BD Transduction labs and Santa Cruz), Actin (Santa Cruz), Flag M2 (Sigma), Arc (Santa Cruz H-300), PSD-95, (Neuromab), EAAT1, EAAT2, TSC2 (Cell Signaling), APP (Epitomics), GabrRα1, β1 and β3, GluR2, Kv1.1, Kv4.2, NR2B (Neuromab), GluR1, EACC1 (Millipore) NR2A (Santa Cruz), and PLIC/Ubiquilin (BD Transduction labs) were used.

Western Blots:

Western blots were run using standard protocols. Protein concentrations of cortical lysates were measured by BCA assay (Pierce) and equal amounts of protein was loaded onto 8% gels, run at 120V, transferred to nitrocellulose, blocked with 4% milk in PBST, and incubated with the primary antibody at 1:1000 to 1:5000 overnight in 4% milk/PBST. Blots were then washed, incubated with the appropriate HRP-conjugated secondary antibody for 1 hour at RT (Santa Cruz), washed, and developed with Femto luminol reagent (Pierce) and images were acquired with a digital camera in a gel dock system (BioRad). Arc protein in barrel cortex was assayed by Western blot of single housed male mice exposed to a novel object for three hours before sacrifice (as in Greer et al. 2010).

In Vitro Ubiquitination Assay:

Cortical lysates were prepared in PBS with 1% TritonX-100 and protease inhibitors, incubated with 4 μg of anti-FLAG antibodies overnight, and with 50 μl of protein G magnetic beads (NEB) for immunoprecipitation (IP). Beads were washed 5× with PBS and Ube3a-FLAG was eluted with 3× FLAG peptide (Sigma) in 100 μl PBS, and IP success was confirmed by western blot. To ubiquitination buffer (in mMol: TRIS 20, NaCl 50, MgCl 10, DTT 0.1, MG132 10, ATP 4 pH 7.4) was added 1 μg recombinant Arc (Novus biologicals), 50 ng E1, 100 ng UbcH7 E2, 4 μg HA-Ubiquitin (all from Boston Biochem) and 10 μl of immunoprecipitate for a total reaction volume of 100 μl (adapted from Greer et al. 2009). Reactions were incubated for 2 hours at 30° before the addition of SDS sample buffer and Western blotting.

Staining:

For tissue sections, mice were perfused with 4% PFA and brains removed and cut into 2 mm pieces which were paraffin embedded. 15 μm sections were cut and mounted and deparaffinized in xylene, re-hydrated through an ethanol gradient, and boiled for 20 minutes in citrate buffer to unmask antigens. Alternately, sections were frozen in OCT and cut on a cryostat at 5, 20 or 100 μm for PSD/VGlut, Ube3a/FLAG, and external GluR1, respectively. Sections were blocked with MOM reagent in the case of anti-mouse secondary (Vector) and then with 10% normal goat serum/1% BSA/0.25-1% Triton X100 in PBS and incubated at room temperature overnight with antibody diluted 1:200 in blocking solution. Sections were then washed, incubated with Alexa-conjugated secondary antibodies (Invitrogen), and mounted in Vectashield with DAPI (Vector). Images were acquired on a LSM510 confocal microscope (Zeiss). For PSD95/Vglut1, confocal image stacks were taken at 63× magnification through the 5 um slice from random positions in layer II/III. Colocalization of Vglut and PSD95 puncta were counted from 3 images for each section, at least 3 sections per animal, n=4 animals.

Tissue Culture:

P0 mice were euthanized and cortical neurons were prepared with a postnatal neuron isolation kit (Miltenyi Biotech) according to the manufactures instructions, and maintained in MACS neuronal culture media (Miltenyi Biotech) supplemented with B27 (Invitrogen). After 7 days, neurons were fixed in cold 4% PFA in PBS, blocked with blocking solution and stained as above.

Golgi Staining was performed using the FD rapid golgi stain kit (FD Neurotech). The number of spines were counted from the last branch point to the end on terminal dendrites of layer 2 pyramidal neurons which fulfilled the following requirements: 1) they were over 30 μm long; 2) terminated within the slice; and 3) were traceable back to a cell body. The length of the terminal dendrites was measured and data were expressed as spines per μm. At least 10 dendrites were counted per mouse and averaged to give the measure for that mouse. Statistics were based on number of mice.

Electron Microscopy:

Brains were removed for staining and ˜1 mm cubes of barrel cortex containing the pial surface were cut and post-fixed in 3% formaldehyde, 3% gluteraldehyde and 0.1M Na-Cacodylate. Ultrathin sections (70-80 nm) were cut and observed on a transmission EM (JEOL, Co. JEM 1011). Glutamatergic (asymmetrical) synapses were counted at 10,000× magnification based on the appearance of a prominent post-synaptic density. 30 fields were counted from each animal and averaged to obtain the value for the animal. The area of synaptic vesicles was traced using ImageJ and the diameter was derived. 8 synapses imaged at 100,000× magnification, each with between 7 and 17 vesicles, were counted per animal and averaged to obtain the value for each animal.

### Behavior Testing

Single-transgenic mice on a pure FVB/NJ background were bred together to produce litters containing wild-type, single and double transgenic littermates that were used for all experiments, except those shown in FIG. 8, in which either male or female transgenic mice that were bred with a wild-type FVB. Mice were housed in same-sex groups of 3-5 under standard laboratory conditions, lights on from 7 am to 7 pm, ad libitum food and water. Testing was performed between 10 am and 5 pm. Each test was separated by at least three days to prevent one test from interfering with the others. All equipment was cleaned with mild detergent in between each mouse to eliminate residual orders. Wild-type, and single- or double-transgenic littermates were always examined.

Pup Tests:

On P3, pups were removed from the nest one at a time and placed in a clean plastic container at room temperature (23±1) with the bat detector from the Ultravox systems (Noldus) mounted in a hole in the lid. Vocalizations were monitored for five minutes using the Ultravox system, which recorded the number of vocalizations and the time spent vocalizing. The pup was then placed on its back and the time to roll over onto all four paws was measured. The pup was then placed head-down on a, wire screen inclined at 30 degrees, and the time the pup took to turn itself so that its head was above horizontal was recorded. The skin temperature of the pup was then monitored with a digital thermometer to ensure a lack of hypothermia. The pup was then weighted, tattooed on the foot for identification, and placed in a holding cage on a 37° heat pad until all pups were tested, at which time the litter was placed back in the nest. The tests were repeated every other day until P11 (inclusive).

All of the Following Tests were Performed on Adults (8-16 Weeks Old) Except the Juvenile Social Interaction which Used 3-4 Week Old Mice.

Open Field:

Mice were placed in a clear acrylic box measuring 50×100 cm on a black surface. An overhead camera recorded activity and Ethovision (Noldus) was used to measure total distance traveled, time spent in the center (defined as the area formed by lines extending from ⅓ and ⅔ of the length of each side) and total entries into the center.

Adult Social Interaction:

As in Smith et al. 2007. Dividers with small (10×10 cm) doors were placed into the open field box to create a three-chambered enclosure. Small cages (metal enclosures, inverted pencil holders, Office Depot) were placed in the upper corners of the outside chambers. Mice were allowed to explore the chambers and small cages for five minutes (during which time they showed no preference for one side over the other). They were then placed in a holding cage, and a same-sex, age-matched, non-littermate. stranger wild-type mouse was placed in one of the two small cages, which were alternated to control for any innate side preference. Mice were recorded with an overhead camera and the time spent in each third of the enclosure, and in the zone immediately next to the enclosure was automatically scored with Ethovision. The test was later repeated with an object (a 10 cm high, 6 cm diameter plastic container, painted with alternating black and white lines) replacing the stranger mouse.

Juvenile Social Interaction:

(As in Crawley 2007) The same three-chambered arena was used. Mice were allowed to explore the empty arena for ten minutes. They were then placed in a holding cage. The small metal enclosures were then placed in the arena, and a same-sex, age-matched, non-littermate wild-type stranger mouse was placed in one of the two small cages, which were alternated to control for any innate side preference. These probe mice had been habituated to the small enclosures in 1 hour sessions for three days prior to testing. Mice were recorded with an overhead camera and the time spent in each third of the enclosure was automatically scored with Ethovision. An observer blinded to genotype of the mouse also scored the time spent interacting with the probe mouse or the empty cage.

Elevated Plus:

Mice were placed, with their heads facing into a closed arm, onto an elevated plus maze 50 cm off of the ground, with 50×5 cm arms and were allowed to explore for five minutes. Mouse behavior was recorded with an overhead camera and the time spent in each arm and the number of entries into each arm was automatically scored with Ethovision.

Object Exploration/Memory:

Mice were placed into the open field box with two of three objects placed in diagonally opposite corners. The mice were allowed to explore the objects for five minutes, after which time they were placed in a holding cage while the arena was cleaned and one of the two objects was replaced with the third “novel” object. After 10 minutes, the mouse was returned to the arena and allowed to explore both objects for a further five minutes. All sessions were recorded by an overhead camera, the video files were coded, and the number of exploratory sniffs to each target (defined as moving the nose to within 3 cm of the object with the head facing the object) was counted by an experienced observer blinded to the genotype of each mouse. The order of object presentation and the location of the object in different diagonal corners were randomized to control for any innate object or location preference, but post-hoc analysis revealed no such preference.

Grooming:

Mice were allowed to acclimate in a clean cage for ten minutes. The total amount of time spent grooming was then recorded with a stopwatch by an experienced observer blinded to the genotype of each mouse. As videotaped recordings were difficult to accurately score, scoring was done live.

Rotorod:

The rotorod (Ugo Basile A-Rod for mice) was set to accelerate from 4 to 40 RPM over five minutes. Time to fall was recorded for each mouse, and if a mouse was still on the rod after 400 seconds, the session was ended and a score of 400 given. Each mouse was given four sessions a day, separated by approximately one hour, for three consecutive days.

Sexual Vocalizations:

Male mice were single-housed for several days, and then exposed to brief (5 min) social interactions with both male and female mice for four days before the test. On the 5^{th} day, mice were placed in a small plastic box inside a larger sound-proof container. A cotton swab dipped in freshly-collected urine pooled from at least 10 females from at least 5 different cages was suspended from the top of the smaller box, so that the tip was approx 5 cm above the floor. An ultrasonic microphone recorded vocalizations and fed data into a computer running Avisoft-Recorder (Avisoft Bioacoustics) for five minutes. The program recorded the total number of vocalizations and time spent vocalizing. The WAV file was then analyzed using SASLab Pro (Avisoft Bioacoustics). A spectrogram was generated and an experienced observer classified each vocalization into one of ten categories. The categories were defined as: “2” a harmonic call where the higher frequency band was dominant; “d” a harmonic call where the lower frequency was dominant; “4” a characteristically shaped 4-part harmonic call; “s” a non-harmonic call with a sharp frequency step; “q” a call that first showed upward frequency modulation, then downward, then upward again in a sinusoidal waveform; “i” a call that showed upward then downward frequency modulation, like an inverted parabola; “p” a call that showed downward then upward frequency modulation, like a parabola; “e” a call that shows upward frequency modulation, then flattens; “f” a flat call; and “u” a call with consistent upwards frequency modulation. N=number of mice tested.

Social Vocalizations:

The same setup which was used for the pup vocalization testing was used for the social vocalization testing because it allowed direct visual monitoring of pairs of mice to ensure fighting did not occur. Procedure was loosely adapted from Scattoni et al (2008). Age- and genotype-matched, non-littermate, female mice who had never encountered each other before were placed in the box simultaneously (to avoid resident-intruder aggression) and the number of vocalizations and time spent vocalizing were recorded automatically (Ultravox, Noldus) for five minutes. Data were not normally distributed, so non-parametric tests were used. N=pairs of mice tested.

Olfactory Habituation/Dishabituation:

One hour before the test, the mouse was acclimated to the swab, suspended from the center of the top of a clean cage to 5 cm above floor level. A fresh swab was then dipped in odorant solution and suspended as above for two minutes. Sessions were video-recorded and an observer blinded to the genotype of the mouse scored the amount of time the mouse spent sniffing the swab. After two minutes, the swab was replaced. Each odorant was presented three times to measure habituation, and four different odorants were presented to measure dis-habituation and the ability of the mice to smell different substances. Odorants were: 1) distilled water; 2) swab was wiped across the bottom of a dirty female cage; 3) 1:10 dilution of imitation banana extract (McCormick); and 4) 1:10 dilution of almond extract (McCormick).

### Electrophysiology

Slice Preparation:

Mice between 8 and 16 weeks old were used for mEPSC, mIPSC, and biophysical properties, and between 4-8 weeks old for all other tests. Cells from at least 3 mice were analyzed, and n was based on number of cells. Testing order was random with respect to genotype. Mice were anaesthetized with 2,2,2,tribromomethanol (0.25 mg/g body weight) and transcardially-perfused with ice-cold sucrose-containing cutting solution (in mM: Sucrose 234, KCl 5, NaH2PO4 1.25, MgSO4 5, NaHCO3 26, Dextrose 25, CaCl2 1, balances with 95% O2/5% CO2). The brain was removed and coronal slices (approx 280 μm) were cut on a tissue slicer (Leica VT1200S) in cutting solution. Barrel cortex was identified as in (Paxinos Atlas). Slices were incubated at 35° C. for 30 min in ACSF (in mM: NaCl 125, KCl-3, NaH2PO4 1.25, MgCl2 1, NaHCO3 26, Dextrose 25, CaCl2 2) before being incubated at room temperature for at least 30 min before recording.

Electrophysiological Recording:

Whole-cell recordings of layer 2/3 pyramidal neurons (PNs) in the barrel cortex were performed on the coronal brain slices. PNs were identified under infrared differential interference contrast (IR-DIC) optics on an upright Olympus BX-51WI microscope (Olympus, Tokyo, Japan) based on their location and morphology. Recording pipettes were pulled from 1.5 mm OD capillary tubing (A-M Systems, Carlsborg, Wash., USA) using a Flaming/Brown P-97 pipette puller (Sutter Instruments, Novato, Calif., USA) and had tip resistances of 3-5 MS2 when filled with internal solution (see below). Pipettes were connected to the headstage of a Heka EPC 10 patch-clamp amplifier (Heka Elektronik) and Patchmaster 2 software (HEKA Instruments, Southboro, Mass., USA) was used. Fast and slow capacitance and series resistance compensations were carefully adjusted. Liquid junction potentials were not corrected. Cells with a resting Vm between −60 and −82 mV and a series resistance <20 MΩ were included for analysis. Recordings with series resistance change exceeding 20% were terminated and discarded. Recordings were filtered at 2.9 kHz and digitized at 50 kHz.

Basic biophysical and firing properties were recorded in current-clamp mode using the following intracellular solution (in mM: 135 KCH3SO3, 4 KCl, 2 NaCl, 10 HEPES, 4 MgATP, 0.3 Tris-GTP, 7 Tris-Phosphocreatine) and regular extracellular ACSF. Input resistance was estimated with a negative square pulse (−25 pA, 200 ms). Membrane time constant was obtained by fitting a single exponential equation to the voltage response to this small negative current pulse. Positive current steps (duration: 1 s or 5 ms) were used to acquire the firing properties.

Evoked postsynaptic currents were recorded in voltage-clamp mode using cesium-based artificial intracellular fluid (in mM: 100 CsCH₃SO₃, 20 KCl, 10 HEPES, 4 Mg-ATP, 0.3 Tris-GTP, 7 Tris₂-Phosphocreatine, 3 QX-314) and regular ACSF. A bipolar platinum/iridium electrode (CE2C55, FHC Inc., Bowdoin, Me.) was placed at layer 2/3 of the barrel cortex 200 μm away from the recording site. Presynaptic axons were stimulated using current pulse stimuli (duration=180 μs, amplitude=10-500 μA, and frequency=0.1 Hz for baseline condition) delivered via a constant-current stimulator. Excitatory postsynaptic currents (EPSCs) were recorded at a holding potential of −50 mV. A glass pipette filled with 0.5 mM bicuculline methiodide (BMI) in ACSF was placed above the soma of the cell being recorded. A small positive pressure was applied to the pipette to establish a stable flow of BMI that locally inhibited GABAergic transmission. Inhibitory postsynaptic currents (IPSCs) were recorded at a holding potential of +10 mV in the presence of bath 10 μM DNQX and 50 μM APV.

Paired-pulse facilitation experiments were carried out to estimate the release probability. The peak amplitude of postsynaptic currents evoked by two identical stimuli separated by 50 ms was measured. The facilitation ratio (the second peak amplitude/the first peak amplitude) was calculated.

Miniature EPSCs (mEPSCs) and miniature IPSCs (mIPSCs) were respectively recorded at −60 mV or −80 mV and +10 mV using cesium-based internal fluid (above) and a low divalent ion ACSF (in mM): 125 NaCl, 3.5 KCl, 1.25 NaH2PO4, 0.5 MgCl2, 26 NaHCO3, 25 Dextrose, 4 MgATP, and 1 CaCl2. AMPA receptor-mediated mEPSCs (AMPA-mEPSCs) were recorded in the presence of 20 APV, 100 μM picrotoxin, and 1 μM TTX. NMDA receptor-mediated mEPSCs (NMDA-mEPSCs) were recorded at −70 mV in the presence of 10 μM DNQX, 100 μM picrotoxin, 20 μM glycine, 0 Mg₂₊, and 1 μM TTX. Continuous data were recorded in 10 sec sweeps, filtered at 1 kHz and sampled at 20 kHz, 300 s of synaptic events were randomly chosen and the total number of events was analyzed. Individual events were counted and analyzed with MiniAnalysis software (Synaptosoft) and custom scripts written in MatLab using amplitude as the main identification parameter and a 5 pA cut-off to account for noise. 50 events were randomly chosen from each cell and combined into the total pool of events for each genotype, and the amplitude and the interevent interval histograms were binned at 1 pA and 0.01 s, respectively. Differences between cumulative histograms were evaluated by the Kolmogorov-Smirnov test. The decay times of AMPA-mEPSCs and NMDA-mEPSCs were fitted using one exponential equations.

Spontaneous EPSCs (sEPSCs) were recorded at a holding potential of −50 mV using the same cesium-based internal fluid and regular extracellular ACSF containing 100 μM picrotoxin. Spontaneous IPSCs (sEPSCs) were recorded at a holding potential of +10 mV using cesium-based internal fluid and regular ACSF containing 10 μM DNQX and 50 μM APV. Analysis was similar to mEPSCs and mEPSCs.

Glutamate Iontophoresis:

The proximal portion of the apical dendrites of layer 2/3 pyramidal neurons in the barrel cortex was exposed by blowing ACSF onto the surface of the slice via ACSF-filled glass pipettes. The pyramidal neurons were then voltage-clamped at −70 mV in the presence of 1 μm TTX and 100 μm picrotoxin. Iontophoretically applied glutamate (10 mM sodium glutamate in 10 mM HEPES, pH 7.4) was delivered through glass pipettes (4-6 MΩ when filled with normal internal solution) placed 1-2 μm away from the main apical shaft (˜15-20 μm from cell body). The iontophoresis pipette was connected to the second channel of a Heka EPC 10 amplifier and glutamate was expelled using 100 ms-, 100 nA current pulses at 0.1 Hz. 1 nA retention currents were applied between stimuli to prevent glutamate leakage in the baseline conditions.

Minimal Stimulation and Estimation of Vesicle Glutamate Content:

The vesicle glutamate content was estimated by the relative inhibition of mean single fiber EPSC amplitude by the fast off-rate, non-NMDA receptor blocker γ-DGG (300 μM). The higher the percentage inhibition by γ-DGG, the lower the concentration of synaptic glutamate (see ref. 21). To selectively stimulate a single fiber in layer 2/3 of the barrel cortex, minimal stimuli were delivered through ACSF-filled bipolar glass electrodes pulled from 2.0 mm OD dual barrel theta capillary glass (Warner Instruments). The tip of the stimulating electrodes is about 2 μm. To establish a minimal stimulation, we first sought for the highest stimulus that gave all failures. Then we slightly increased the stimulation intensity to lower the failure rate. To acquire a reasonable number of EPSCs from 40-100 trials in both baseline and γ-DGG conditions at 0.3 Hz, we adjusted the stimulation intensity to give about 10% failures (WT: 9.7±2.4%, n=8; 1×: 9.7±3.4%, n=8; 2×: 11.6±4.6%, n=6; F(2, 21)=0.106, P=0.90). Under this failure rate, the calculated quantal content was about 2. In addition, EPSC latency should remain stable throughout the experiments (<20% fluctuations). The other recording conditions were similar to evoked EPSC recordings.

Estimation of Readily Releasable Pool Size and Release Probability.

We used 20 Hz train stimulations (40 stimuli) to estimate the size of readily releasable pool. We averaged 10-20 train stimulations (train frequency: 0.033-0.067 Hz). To effectively discharge the readily releasable pool, a slightly higher stimulation intensity than the afore-mentioned minimal stimulation was used (˜5% more than the minimal stimulation). This stimulation intensity gave <5% failures. The readily releasable pool size was estimated by linear interpolating the linear portion (normally from 21st to 40th stimuli) of the cumulative EPSC amplitude plot to virtual stimulus 0. The ratio of this readily releasable pool size and the quantal size gave the number of readily releasable sites. To estimate the release probability, the mean amplitude of the 1st EPSC during the train stimulation was divided by the readily releasable pool size. The other recording conditions were similar to evoked EPSC recordings.

### Statistical Testing

Behavioral data were analyzed using Prizm (Graphpad). Comparisons between two groups used Student's T-Test, comparisons among multiple groups used one-way ANOVA with Dunnett's post-hoc test comparing each genotype to wild-type; non-significant comparisons are not stated in the manuscript. Comparisons involving multiple independent variables used two-way ANOVAs. Non-normal data (social vocalizations) were tested using the Kruskal-Wallis test followed by Dunn's multiple comparison post-hoc tests comparing each genotype to wild-type.

For electrophysiological data, one-way ANOVA with Dunnett's post-hoc test was used to compare multiple group means. Kolmogorov-Smirnov (KS) test was used to compare distributions. Unbalanced two-way ANOVA with bonferroni's post-hoc test was used to compare multiple group variance. n=number of cells analyzed.

All data is presented as mean±SEM unless otherwise noted. P<0.05 was considered statistically significant.

TABLE 1
(Statistical Table)
Three-chamber tests

| | | | Target | Middle | Opposite | N | t value | | |
|---|---|---|---|---|---|---|---|---|---|
| Juvenile Social | Time interacting (s) | Wt | 203.3 ± 19.47 | — | 138.7 ± 14.08 | 11 | t = 2.639 df = 20 | p = 0.0157 | ttest comparing time spent in ″target″ vs ″opposite″ zones |
| Interaction | | 1 × Tg | 176.9 ± 12.38 | — | 140.2 ± 7.83 | 15 | t = 2.499 df = 28 | p = 0.0186 | ttest comparing time spent in ″target″ vs ″opposite″ zones |
| (25-32 days) | | 2 × Tg | 138.3 ± 10.56 | — | 167.5 ± 15.90 | 12 | t = 1.531 df = 22 | p = 0.1399 | ttest comparing time spent in ″target″ vs ″opposite″ zones |
| | Time in Zone (s) | Wt | 288.7 ± 19.91 | 89.2 ± 10.35 | 222.1 ± 15.77 | 11 | t = 2.625 df = 20 | p = 0.0162 | ttest comparing time spent in ″target″ vs ″opposite″ zones |
| | | 1 × Tg | 258.2 ± 10.76 | 102.0 ± 10.19 | 239.8 ± 10.26 | 15 | t = 1.237 df = 28 | p = 0.2264 | ttest comparing time spent in ″target″ vs ″opposite″ zones |
| | | 2 × Tg | 235.1 ± 9.86 | 120.3 ± 9.83 | 244.7 ± 14.71 | 12 | t = 0.5459 df = 22 | p = 0.5907 | ttest comparing time spent in ″target″ vs ″opposite″ zones |
| Adult Social | Time in Zone (s) | Wt | 151.2 ± 5.3 | 74.1 ± 5.4 | 74.7 ± 7.3 | 17 | t = 8.481 df = 32 | p < 0.0001 | ttest comparing time spent in ″target″ vs ″opposite″ zones |
| Interaction | | 1 × Tg | 142.4 ± 8.7 | 78.4 ± 14.6 | 79.20 ± 10.0 | 10 | t = 4.773 df = 18 | p = 0.0002 | ttest comparing time spent in ″target″ vs ″opposite″ zones |
| (8-10 weeks) | | 2 × Tg | 120.0 ± 11.8 | 63.7 ± 6.1 | 116.3 ± 12.5 | 15 | t = 0.2167 df = 28 | p = 0.8300 | ttest comparing time spent in ″target″ vs ″opposite″ zones |
| | | 2 × | 175.0 ± 21.0 | 54.4 ± 6.3 | 70.6 ± 15.5 | 6 | t = 3.998 df = 10 | p = 0.0025 | ttest comparing time spent in ″target″ vs ″opposite″ zones |
| | | Inactive Tg | | | | | | | |
| Object Interaction | Time in Zone (s) | Wt | 126.1 ± 8.4 | 76.0 ± 6.0 | 98.0 ± 8.5 | 11 | t = 2.340 df = 20 | p = 0.0297 | ttest comparing time spent in ″target″ vs ″opposite″ zones |
| (9-11 weeks) | | 2 × Tg | 141.4 ± 10.1 | 57.9 ± 5.2 | 100.7 ± 9.9 | 13 | t = 2.878 df = 24 | p = 0.0083 | ttest comparing time spent in ″target″ vs ″opposite″ zones |

Other behavior tests

| | | Mean +/− SEM | | | | | | | | | |
|---|---|---|---|---|---|---|---|---|---|---|---|
| Test | Parameter | Wt | 1 × Tg | 2 × Tg | 2 × Inactive | Test statistic | P Value | Test details | | | |
| Grooming | Time spent grooming (s) | 10.3 ± 2.1 | 13.4 ± 2.1 | 28.7 ± 7.0 | 13.6 ± 3.6 | F(3.42) = 4.073 | p = 0.0126 | 1-way ANOVA | Dunnett′s multiple | wt vs 1 × Tg | P > 0.05 |
| 12-14 weeks | N | 12 | 12 | 11 | 11 | | | | comparison | wt vs 2 × Tg | P < 0.01 |
| | | | | | | | | | post-hoc: | wt vs 2 × | P > 0.05 |
| | | | | | | | | | | Inactive | |
| Elevated plus | Entries: open arms | 15.5 ± 2.5 | — | 13.2 ± 1.4 | — | t = 0.7420 df = 22 | p = 0.466 | ttest, wt vs 2 × Tg | | | |
| 10-12 weeks | Entries: closed arms | 22.5 ± 1.8 | — | 23.3 ± 1.2 | — | t = 0.4083 df = 22 | p = 0.6870 | ttest, wt vs 2 × Tg | | | |
| | Time spent: open arms (s) | 66.5 ± 11.4 | — | 51.8 ± 5.1 | — | t = 1.244 df = 22 | p = 0.2266 | ttest, wt vs 2 × Tg | | | |
| | Time spent: closed arms (s) | 146.7 ± 12.9 | — | 162.4 ± 7.1 | — | t = 1.114 df = 22 | p = 0.2773 | ttest, wt vs 2 × Tg | | | |
| | Ratio open/total entries | 0.385 ± 0.043 | — | 0.352 ± 0.024 | — | t = 0.7124 df = 22 | p = 0.4837 | ttest, wt vs 2 × Tg | | | |
| | Ratio open/total time | 0.313 ± 0.056 | — | 0.244 ± 0.025 | — | t = 1.186 df = 22 | p = 0.2481 | ttest, wt vs 2 × Tg | | | |
| | N | 11 | | 13 | | | | | | | |
| Open Field | Distance Traveled (cm) | 5612 ± 165.0 | — | 5369 ± 310.3 | — | t = 0.6514 df = 20 | p = 0.5222 | ttest, wt vs 2 × Tg | | | |
| 11-13 weeks | Entries into center | 37.7 ± 2.4 | — | 31.5 ± 4.0 | — | t = 1.252 df = 20 | p = 0.2250 | ttest, wt vs 2 × Tg | | | |
| | Time in center (s) | 31.6 ± 2.3 | — | 29.6 ± 3.3 | — | t = 0.4889 df = 20 | p = 0.6303 | ttest, wt vs 2 × Tg | | | |
| | N | 10 | | 12 | | | | | | | |
| Object recog- | Baseline: touches of object A | 12.3 ± 1.3 | — | 8.8 ± 1.4 | — | t = 0.5680 df = 20 | p = 0.5764 | ttest, wt vs 2 × Tg | | | |
| nition test | Baseline: touches of object B | 11.2 ± 1.4 | — | 9.9 ± 1.1 | — | t = 0.6173 df = 22 | p = 0.5434 | ttest, wt vs 2 × Tg | | | |
| 12-13 weeks | Baseline: Total touches | 23.5 ± 2.4 | — | 18.8 ± 2.3 | — | t = 1.420 df = 21 | p = 0.1703 | ttest, wt vs 2 × Tg | | | |
| | Memory: touches of object A | 8.9 ± 0.9 | — | 7.9 ± 0.7 | — | t = 3.671 df = 38 | p = 0.0007 | ttest, wt vs 2 × Tg | | | |
| | Memory: Touches of object C | 13.9 ± 1.0 | — | 10.8 ± 0.9 | — | t = 2.562 df = 40 | p = 0.0143 | ttest, wt vs 2 × Tg | | | |
| | Memory: Total touches | 22.1 ± 1.6 | — | 18.7 ± 1.8 | — | t = 1.445 df = 32 | p = 0.1583 | ttest, wt vs 2 × Tg | | | |
| | N | 20 | | 21 | | | | | | | |
| Rotorod | Latency to fall: day 1 | 197.9 ± 74.2 | 171.0 ± 37.9 | 163.7 ± 39.7 | 210.7 ± 63.4 | 2-way ANOVA, Genotype | | F(3.204) = 0.1154 | p = 0.951 | | |
| (3-4 months) | Latency to fall: day 2 | 270.5 ± 90.5 | 295.9 ± 69.4 | 242.9 ± 69.6 | 242.9 ± 62.9 | 2-way ANOVA, Day | | F(2.204) = 47.5 | p = P < 0.0001 | | |
| | Latency to fall: day 3 | 299.7 ± 84.5 | 312.4 ± 58.6 | 317.9 ± 70.4 | 335.1 ± 61.5 | 2-way ANOVA, interaction | | F(6.204) = 1.574 | p = 0.1564 | | |
| | | 31 | 14 | 20 | 7 | | | | | | |
| Urine-induced | Number of vocalizations | 476.9 ± 117.3 | 175.5 ± 61.1 | 186.6 ± 47.9 | — | F(2.22) = 4.515 | p = 0.0228 | 1-way ANOVA | Dunnett′s multiple | wt vs 1 × Tg | p < 0.05 |
| vocalizations | | | | | | | | | comparison | wt vs 2 × Tg | p < 0.05 |
| | | | | | | | | | post-hoc: | | |
| 2-3 months; | Time spent vocalizing | 17.8 ± 5.1 | 5.8 ± 2.0 | 5.2 ± 1.3 | — | F(2.22) = 5.306 | p = 0.0132 | 1-way ANOVA | Dunnett′s multiple | wt vs 1 × Tg | p < 0.05 |
| Males only | N | 7 | 11 | 7 | | | | | comparison | wt vs 2 × Tg | p < 0.05 |
| | | | | | | | | | post-hoc: | | |
| Social | Number of vocalizations | | | | | | | | | | |
| Vocalizations | Total | 80.3 ± 16.3 | 84.9 ± 23.6 | 29.2 ± 6.9 | — | H = 7.76, df = 2 | p = 0.0206 | Kruskal wallis test | Dunn′s multiple | wt vs 1 × Tg | p > 0.05 |
| 2-3 months | N (pairs of animals) | 24 | 15 | 16 | | | | | comparison | wt vs 2 × Tg | p < 0.05 |
| | | | | | | | | | post-hoc | | |
| | Males | 55.1 ± 18.6 | 23.0 ± 8.3 | 32.4 ± 12.2 | — | H = 3.01, df = 2 | p = 0.2225 | Kruskal wallis test | no post-hoc | | |
| | n | 10 | 7 | 8 | | | | | | | |
| | Females | 98.3 ± 23.9 | 139.1 ± 33.6 | 26.0 ± 7.2 | — | H = 10.34, df = 2 | p = 0.0057 | Kruskal wallis test | Dunn′s multiple | wt vs 1 × Tg | p > 0.05 |
| | | 14 | 8 | 8 | | | | | comparison | wt vs 2 × Tg | p < 0.05 |
| | | | | | | | | | post-hoc | | |
| | | | | | | 2-way ANOVA, Genotype | | F(2.49) = 3.318 | p = 0.0445 | | |
| | | | | | | 2-way ANOVA, Sex | | F(1.49) = 7.812 | p = 0.0074 | | |
| | | | | | | 2-way ANOVA, Interaction | | F(2.49) = 3.805 | p = 0.0291 | | |
| | Time spent vocalizing (S) | | | | | | | | | | |
| | Total | 14.56 ± 4.6 | 18.74 ± 7.6 | 2.95 ± 1.0 | — | H = 5.48, df = 2 | p = 0.0645 | Kruskal wallis test | no post-hoc | | |
| | Males | 6.34 ± 3.3 | 1.81 ± 0.8 | 3.79 ± 1.9 | — | H = 2.89, df = 2 | p = 0.2354 | Kruskal wallis test | no post-hoc | | |
| | Females | 20.42 ± 7.1 | 33.6 ± 12.1 | 2.1 ± 0.6 | — | H = 9.10, df = 2 | p = 0.0106 | Kruskal wallis test | Dunn′s multiple | wt vs 1 × Tg | p > 0.05 |
| | | | | | | | | | comparison | wt vs 2 × Tg | p < 0.05 |
| | | | | | | | | | post-hoc | | |
| | | | | | | 2-way ANOVA, Genotype | | F(2.49) = 2.606 | p = 0.0841 | | |
| | | | | | | 2-way ANOVA, Sex | | F(1.49) = 7.376 | p = 0.0091 | | |
| | | | | | | 2-way ANOVA, Interaction | | F(2.49) = 3.175 | p = 0.0505 | | |
| Pup | P3 (n = 16, 24, 15) | 8.13 ± 0.53 | 9.34 ± 0.32 | 9.64 ± 0.41 | — | 2-way ANOVA, Age | | F(4.262) = 11.55 | p = 0.0001 | | |
| vocalizations | P5 (n = 16, 24, 15) | 27.4 ± 1.03 | 31.55 ± 0.85 | 21.26 ± 1.31 | — | 2-way ANOVA, Genotype | | F(2.262) = 0.8747 | p = 0.4182 | | |
| | P7 (n = 16, 24, 15) | 39.45 ± 1.5 | 35.99 ± 1.13 | 41.02 ± 1.55 | — | 2-way ANOVA, Interaction | | F(8.262) = 0.4107 | p = 0.914 | | |
| | P9 (n = 16, 24, 15) | 28.75 ± 1.15 | 31.67 ± 0.65 | 23.53 ± 0.72 | — | | | | | | |
| | P11 (n = 6,10, 6) | 22.18 ± 5.29 | 21.98 ± 2.31 | 12.7 ± 3.64 | — | | | | | | |
| Olfactory | Time Sniffing q-tip (s) | | | | | | | | | | |
| Habituation/ | Water 1 | 2.38 ± 0.51 | — | 3.48 ± 0.37 | — | | | | | | |
| dishabituation | Water 2 | 1.65 ± 0.65 | — | 1.11 ± 0.19 | — | | | | | | |
| 10-12 weeks | Water 3 | 0.49 ± 0.19 | — | 1.02 ± 0.48 | — | 2-way ANOVA, Genotype | | F(1.132) = 2.723 | p = 0.1248 | | |
| | Cage 1 | 15.8 ± 3.08 | — | 20.24 ± 2.62 | — | 2-way ANOVA, Session | | F(11.132) = 27.45 | p < 0.0001 | | |
| | Cage 2 | 6.9 ± 2.5 | — | 8.39 ± 3.33 | — | 2-way ANOVA, Interaction | | F(11.132) = 0.5377 | p = 0.8747 | | |
| | Cage 3 | 2.71 ± 1.23 | — | 5.14 ± 2.24 | — | 2-way ANOVA, Subjects | | F(12.132) = 2.868 | p = 0.0015 | | |
| | | | | | | (matching) | | | | | |
| | Banana 1 | 2.98 ± 0.6 | — | 6.51 ± 1.22 | — | | | | | | |
| | Banana 2 | 1.28 ± 0.69 | — | 1.79 ± 0.49 | — | | | | | | |
| | Banana 3 | 0.77 ± 0.32 | — | 1.73 ± 0.46 | — | | | | | | |
| | Almond 1 | 6.01 ± 1.81 | — | 8.1 ± 0.92 | — | | | | | | |
| | Almond 2 | 1.04 ± 0.56 | — | 1.67 ± 0.55 | — | | | | | | |
| | Almond 3 | 0.39 ± 0.19 | — | 1.57 ± 0.56 | — | | | | | | |
| | N | 7 | | 7 | | | | | | | |

### REFERENCES

1. S. E. Levy, D. S. Mandell, R. T. Schultz, Autism. Lancet 374, 1627-1638 (2009).
2. B. S. Abrahams, D. H. Geschwind, Advances in autism genetics: on the threshold of a new neurobiology. Nat Rev Genet 9, 341-355 (2008).
3. J. T. Glessner, K. Wang, G. Cai, O. Korvatska, C. E. Kim, S. Wood, H. Zhang, A. Estes, C. W. Brune, J. P. Bradfield, M. Imielinski, E. C. Frackelton, J. Reichert, E. L. Crawford, J. Munson, P. M. Sleiman, R. Chiavacci, K. Annaiah, K. Thomas, C. Hou, W. Glaberson, J. Flory, F. Otieno, M. Garris, L. Soorya, L. Klei, J. Piven, K. J. Meyer, E. Anagnostou, T. Sakurai, R. M. Game, D. S. Rudd, D. Zurawiecki, C. J. McDougle, L. K. Davis, J. Miller, D. J. Posey, S. Michaels, A. Kolevzon, J. M. Silverman, R. Bernier, S. E. Levy, R. T. Schultz, G. Dawson, T. Owley, W. M. McMahon, T. H. Wassink, J. A. Sweeney, J. I. Nurnberger, H. Coon, J. S. Sutcliffe, N. J. Minshew, S. F. Grant, M. Bucan, E. H. Cook, J. D. Buxbaum, B. Devlin, G. D. Schellenberg, H. Hakonarson, Autism genome-wide copy number variation reveals ubiquitin and neuronal genes. Nature 459, 569-573 (2009).
4. E. M. Morrow, S. Y. Yoo, S. W. Flavell, T. K. Kim, Y. Lin, R. S. Hill, N. M. Mukaddes, S. Balkhy, G. Gascon, A. Hashmi, S. Al-Saad, J. Ware, R. M. Joseph, R. Greenblatt, D. Gleason, J. A. Ertelt, K. A. Apse, A. Bodell, J. N. Partlow, B. Barry, H. Yao, K. Markianos, R. J. Ferland, M. E. Greenberg, C. A. Walsh, Identifying autism loci and genes by tracing recent shared ancestry. Science 321, 218-223 (2008).
5. D. Pinto, A. T. Pagnamenta, L. Klei, R. Anney, D. Merico, R. Regan, J. Conroy, T. R. Magalhaes, C. Correia, B. S. Abrahams, J. Almeida, E. Bacchelli, G. D. Bader, A. J. Bailey, G. Baird, A. Battaglia, T. Berney, N. Bolshakova, S. Bolte, P. F. Bolton, T. Bourgeron, S. Brennan, J. Brian, S. E. Bryson, A. R. Carson, G. Casallo, J. Casey, B. H. Chung, L. Cochrane, C. Corsello, E. L. Crawford, A. Crossett, C. Cytrynbaum, G. Dawson, M. de Jonge, R. Delorme, I. Drmic, E. Duketis, F. Duque, A. Estes, P. Farrar, B. A. Fernandez, S. E. Folstein, E. Fombonne, C. M. Freitag, J. Gilbert, C. Gillberg, J. T. Glessner, J. Goldberg, A. Green, J. Green, S. J. Guter, H. Hakonarson, E. A. Heron, M. Hill, R. Holt, J. L. Howe, G. Hughes, V. Hus, R. Igliozzi, C. Kim, S. M. Klauck, A. Kolevzon, O. Korvatska, V. Kustanovich, C. M. Lajonchere, J. A. Lamb, M. Laskawiec, M. Leboyer, A. Le Couteur, B. L. Leventhal, A. C. Lionel, X. Q. Liu, C. Lord, L. Lotspeich, S. C. Lund, E. Maestrini, W. Mahoney, C. Mantoulan, C. R. Marshall, H. McConachie, C. J. McDougle, J. McGrath, W. M. McMahon, A. Merikangas, O. Migita, N. J. Minshew, G. K. Mirza, J. Munson, S. F. Nelson, C. Noakes, A. Noor, G. Nygren, G. Oliveira, K. Papanikolaou, J. R. Parr, B. Parrini, T. Paton, A. Pickles, M. Pilorge, J. Piven, C. P. Ponting, D. J. Posey, A. Poustka, F. Poustka, A. Prasad, J. Ragoussis, K. Renshaw, J. Rickaby, W. Roberts, K. Roeder, B. Roge, M. L. Rutter, L. J. Bierut, J. P. Rice, J. Salt, K. Sansom, D. Sato, R. Segurado, A. F. Sequeira, L. Senman, N. Shah, V. C. Sheffield, L. Soorya, I. Sousa, O. Stein, N. Sykes, V. Stoppioni, C. Strawbridge, R. Tancredi, K. Tansey, B. Thiruvahindrapduram, A. P. Thompson, S. Thomson, A. Tryfon, J. Tsiantis, H. Van Engeland, J. B. Vincent, F. Volkmar, S. Wallace, K. Wang, Z. Wang, T. H. Wassink, C. Webber, R. Weksberg, K. Wing, K. Wittemeyer, S. Wood, J. Wu, B. L. Yaspan, D. Zurawiecki, L. Zwaigenbaum, J. D. Buxbaum, R. M. Cantor, E. H. Cook, H. Coon, M. L. Cuccaro, B. Devlin, S. Ennis, L. Gallagher, D. H. Geschwind, M. Gill, J. L. Haines, J. Hallmayer, J. Miller, A. P. Monaco, J. I. Nurnberger, Jr., A. D. Paterson, M. A. Pericak-Vance, G. D. Schellenberg, P. Szatmari, A. M. Vicente, V. J. Vieland, E. M. Wijsman, S. W. Scherer, J. S. Sutcliffe, C. Betancur, Functional impact of global rare copy number variation in autism spectrum disorders. Nature 466, 368-372 (2010).
6. J. Sebat, B. Lakshmi, D. Malhotra, J. Troge, C. Lese-Martin, T. Walsh, B. Yamrom, S. Yoon, A. Krasnitz, J. Kendall, A. Leotta, D. Pai, R. Zhang, Y. H. Lee, J. Hicks, S. J. Spence, A. T. Lee, K. Puura, T. Lehtimaki, D. Ledbetter, P. K. Gregersen, J. Bregman, J. S. Sutcliffe, V. Jobanputra, W. Chung, D. Warburton, M. C. King, D. Skuse, D. H. Geschwind, T. C. Gilliam, K. Ye, M. Wigler, Strong association of de novo copy number mutations with autism. Science 316, 445-449 (2007).
7. L. A. Weiss, Y. Shen, J. M. Korn, D. E. Arking, D. T. Miller, R. Fossdal, E. Saemundsen, H. Stefansson, M. A. Ferreira, T. Green, O. S. Platt, D. M. Ruderfer, C. A. Walsh, D. Altshuler, A. Chakravarti, R. E. Tanzi, K. Stefansson, S. L. Santangelo, J. F. Gusella, P. Sklar, B. L. Wu, M. J. Daly, Association between microdeletion and microduplication at 16p11.2 and autism. N Engl J Med 358, 667-675 (2008).
8. A. Hogart, D. Wu, J. M. LaSalle, N. C. Schanen, The comorbidity of autism with the genomic disorders of chromosome 15q11.2-q13. Neurobiol Dis 38, 181-191 (2010).
9. U. Albrecht, J. S. Sutcliffe, B. M. Cattanach, C. V. Beechey, D. Armstrong, G. Eichele, A. L. Beaudet, Imprinted expression of the murine Angelman syndrome gene, Ube3a, in hippocampal and Purkinje neurons. Nat Genet 17, 75-78 (1997).
10. Y. H. Jiang, D. Armstrong, U. Albrecht, C. M. Atkins, J. L. Noebels, G. Eichele, J. D. Sweatt, A. L. Beaudet, Mutation of the Angelman ubiquitin ligase in mice causes increased cytoplasmic p53 and deficits of contextual learning and long-term potentiation. Neuron 21, 799-811 (1998).
11. T. Kishino, M. Lalande, J. Wagstaff, UBE3A/E6-AP mutations cause Angelman syndrome. Nat Genet 15, 70-73 (1997).
12. S. V. Dindot, B. A. Antalffy, M. B. Bhattacharjee, A. L. Beaudet, The Angelman syndrome ubiquitin ligase localizes to the synapse and nucleus, and maternal deficiency results in abnormal dendritic spine morphology. Hum Mol Genet 17, 111-118 (2008).
13. Y. H. Jiang, Y. Pan, L. Zhu, L. Landa, J. Yoo, C. Spencer, I. Lorenzo, M. Brilliant, J. Noebels, A. L. Beaudet, Altered ultrasonic vocalization and impaired learning and memory in Angelman syndrome mouse model with a large maternal deletion from Ube3a to Gabrb3. PLoS One 5, e12278 (2010).
14. E. J. Weeber, Y. H. Jiang, Y. Elgersma, A. W. Varga, Y. Carrasquillo, S. E. Brown, J. M. Christian, B. Mirnikjoo, A. Silva, A. L. Beaudet, J. D. Sweatt, Derangements of hippocampal calcium/calmodulin-dependent protein kinase II in a mouse model for Angelman mental retardation syndrome. J Neurosci 23, 2634-2644 (2003).
15. G. M. van Woerden, K. D. Harris, M. R. Hojjati, R. M. Gustin, S. Qiu, R. de Avila Freire, Y. H. Jiang, Y. Elgersma, E. J. Weeber, Rescue of neurological deficits in a mouse model for Angelman syndrome by reduction of alphαCaMKII inhibitory phosphorylation. Nat Neurosci 10, 280-282 (2007).
16. M. Sato, M. P. Stryker, Genomic imprinting of experience-dependent cortical plasticity by the ubiquitin ligase gene Ube3a. Proc Natl Acad Sci USA 107, 5611-5616 (2010).
17. K. Yashiro, T. T. Riday, K. H. Condon, A. C. Roberts, D. R. Bernardo, R. Prakash, R. J. Weinberg, M. D. Ehlers, B. D. Philpot, Ube3a is required for experience-dependent maturation of the neocortex. Nat Neurosci 12, 777-783 (2009).
18. M. Scheffner, J. M. Huibregtse, R. D. Vierstra, P. M. Howley, The HPV-16 E6 and E6-AP complex functions as a ubiquitin-protein ligase in the ubiquitination of p53. Cell 75, 495-505 (1993).
19. P. L. Greer, R. Hanayama, B. L. Bloodgood, A. R. Mardinly, D. M. Lipton, S. W. Flavell, T. K. Kim, E. C. Griffith, Z. Waldon, R. Maehr, H. L. Ploegh, S. Chowdhury, P. F. Worley, J. Steen, M. E. Greenberg, The Angelman Syndrome protein Ube3A regulates synapse development by ubiquitinating arc. Cell 140, 704-716 (2010).
20. S. S. Margolis, J. Salogiannis, D. M. Lipton, C. Mandel-Brehm, Z. P. Wills, A. R. Mardinly, L. Hu, P. L. Greer, J. B. Bikoff, H. Y. Ho, M. J. Soskis, M. Sahin, M. E. Greenberg, EphB-mediated degradation of the RhoA GEF Ephexin5 relieves a developmental brake on excitatory synapse formation. Cell 143, 442-455 (2010).
21. Y. D. Zhou, S. Lee, Z. Jin, M. Wright, S. E. Smith, M. P. Anderson, Arrested maturation of excitatory synapses in autosomal dominant lateral temporal lobe epilepsy. Nat Med 15, 1208-1214 (2009).
22. M. Landers, D. L. Bancescu, E. Le Meur, C. Rougeulle, H. Glatt-Deeley, C. Brannan, F. Muscatelli, M. Lalande, Regulation of the large (approximately 1000 kb) imprinted murine Ube3a antisense transcript by alternative exons upstream of Snurf/Snrpn. Nucleic Acids Res 32, 3480-3492 (2004).
23. R. M. Gustin, T. J. Bichell, M. Bubser, J. Daily, I. Filonova, D. Mrelashvili, A. Y. Deutch, R. J. Colbran, E. J. Weeber, K. F. Haas, Tissue-specific variation of Ube3a protein expression in rodents and in a mouse model of Angelman syndrome. Neurobiol Dis 39, 283-291 (2010).
24. J. N. Crawley, Mouse behavioral assays relevant to the symptoms of autism. Brain Pathol 17, 448-459 (2007).
25. S. E. Smith, J. Li, K. Garbett, K. Mimics, P. H. Patterson, Maternal immune activation alters fetal brain development through interleukin-6. J Neurosci 27, 10695-10702 (2007).
26. T. E. Holy, Z. Guo, Ultrasonic songs of male mice. PLoS Biol 3, e386 (2005).
27. G. Ehret, Infant rodent ultrasounds—a gate to the understanding of sound communication. Behav Genet 35, 19-29 (2005).
28. J. Blundell, C. A. Blaiss, M. R. Etherton, F. Espinosa, K. Tabuchi, C. Walz, M. F. Bolliger, T. C. Sudhof, C. M. Powell, Neuroligin-1 deletion results in impaired spatial memory and increased repetitive behavior. J Neurosci 30, 2115-2129 (2010).
29. M. R. Etherton, C. A. Blaiss, C. M. Powell, T. C. Sudhof, Mouse neurexin-1alpha deletion causes correlated electrophysiological and behavioral changes consistent with cognitive impairments. Proc Natl Acad Sci USA 106, 17998-18003 (2009).
30. K. Tabuchi, J. Blundell, M. R. Etherton, R. E. Hammer, X. Liu, C. M. Powell, T. C. Sudhof, A neuroligin-3 mutation implicated in autism increases inhibitory synaptic transmission in mice. Science 318, 71-76 (2007).
31. H. T. Chao, H. Chen, R. C. Samaco, M. Xue, M. Chahrour, J. Yoo, J. L. Neul, S. Gong, H. C. Lu, N. Heintz, M. Ekker, J. L. Rubenstein, J. L. Noebels, C. Rosenmund, H. Y. Zoghbi, Dysfunction in GABA signalling mediates autism-like stereotypies and Rett syndrome phenotypes. Nature 468, 263-269 (2010).
32. G. Liu, S. Choi, R. W. Tsien, Variability of neurotransmitter concentration and nonsaturation of postsynaptic AMPA receptors at synapses in hippocampal cultures and slices. Neuron 22, 395-409 (1999).
33. K. K. Chadman, S. Gong, M. L. Scattoni, S. E. Boltuck, S. U. Gandhy, N. Heintz, J. N. Crawley, Minimal aberrant behavioral phenotypes of neuroligin-3 R451C knockin mice. Autism Res 1, 147-158 (2008).
34. C. Bakker, C. Verheij, T. D.-B. F. X. Consorthium, Fmr1 knockout mice: a model to study fragile X mental retardation. The Dutch-Belgian Fragile X Consortium. Cell 78, 23-33 (1994).
35. M. Shahbazian, J. Young, L. Yuva-Paylor, C. Spencer, B. Antalffy, J. Noebels, D. Armstrong, R. Paylor, H. Zoghbi, Mice with truncated MeCP2 recapitulate many Rett syndrome features and display hyperacetylation of histone H3. Neuron 35, 243-254 (2002).
36. H. G. McFarlane, G. K. Kusek, M. Yang, J. L. Phoenix, V. J. Bolivar, J. N. Crawley, Autism-like behavioral phenotypes in BTBR T+tf/J mice. Genes Brain Behav 7, 152-163 (2008).
37. A. Kashiwagi, M. Meguro, H. Hoshiya, M. Haruta, F. Ishino, T. Shibahara, M. Oshimura, Predominant maternal expression of the mouse Atp10c in hippocampus and olfactory bulb. J Hum Genet 48, 194-198 (2003).
38. M. Meguro, A. Kashiwagi, K. Mitsuya, M. Nakao, I. Kondo, S. Saitoh, M. Oshimura, A novel maternally expressed gene, ATP10C, encodes a putative aminophospholipid translocase associated with Angelman syndrome. Nat Genet 28, 19-20 (2001).
39. T. Kayashima, K. Yamasaki, K. Joh, T. Yamada, T. Ohta, K. Yoshiura, N. Matsumoto, Y. Nakane, T. Mukai, N. Niikawa, T. Kishino, Atp10a, the mouse ortholog of the human imprinted ATP10A gene, escapes genomic imprinting. Genomics 81, 644-647 (2003).
40. J. Nakatani, K. Tamada, F. Hatanaka, S. Ise, H. Ohta, K. Inoue, S. Tomonaga, Y. Watanabe, Y. J. Chung, R. Banerjee, K. Iwamoto, T. Kato, M. Okazawa, K. Yamauchi, K. Tanda, K. Takao, T. Miyakawa, A. Bradley, T. Takumi, Abnormal behavior in a chromosome-engineered mouse model for human 15q11-13 duplication seen in autism. Cell 137, 1235-1246 (2009).
41. Y. Nishimura, C. L. Martin, A. Vazquez-Lopez, S. J. Spence, A. I. Alvarez-Retuerto, M. Sigman, C. Steindler, S. Pellegrini, N. C. Schanen, S. T. Warren, D. H. Geschwind, Genome-wide expression profiling of lymphoblastoid cell lines distinguishes different forms of autism and reveals shared pathways. Hum Mol Genet 16, 1682-1698 (2007).
42. M. R. Kasten, B. Rudy, M. P. Anderson, Differential regulation of action potential firing in adult murine thalamocortical neurons by Kv3.2, Kv1, and SK potassium and N-type calcium channels. J Physiol 584, 565-582 (2007).
43. J. D. Shepherd, G. Rumbaugh, J. Wu, S. Chowdhury, N. Plath, D. Kuhl, R. L. Huganir, P. F. Worley, Arc/Arg3.1 mediates homeostatic synaptic scaling of AMPA receptors. Neuron 52, 475-484 (2006).
44. A. Mishra, N. R. Jana, Regulation of turnover of tumor suppressor p53 and cell growth by E6-AP, a ubiquitin protein ligase mutated in Angelman mental retardation syndrome. Cell Mol Life Sci 65, 656-666 (2008).
45. S. A. Mulherkar, J. Sharma, N. R. Jana, The ubiquitin ligase E6-AP promotes degradation of alpha-synuclein. J Neurochem 110, 1955-1964 (2009).
46. F. Ferdousy, W. Bodeen, K. Summers, O. Doherty, O. Wright, N. Elsisi, G. Hilliard, J. M. O'Donnell, L. T. Reiter, Drosophila Ube3a regulates monoamine synthesis by increasing GTP cyclohydrolase I activity via a non-ubiquitin ligase mechanism. Neurobiol Dis 41, 669-677 (2011).
47. L. T. Reiter, T. N. Seagroves, M. Bowers, E. Bier, Expression of the Rho-GEF Pb1/ECT2 is regulated by the UBE3A E3 ubiquitin ligase. Hum Mol Genet 15, 2825-2835 (2006).
48. Z. Nawaz, D. M. Lonard, C. L. Smith, E. Lev-Lehman, S. Y. Tsai, M. J. Tsai, B. W. O'Malley, The Angelman syndrome-associated protein, E6-AP, is a coactivator for the nuclear hormone receptor superfamily. Mol Cell Biol 19, 1182-1189 (1999).
49. S. Ramamoorthy, Z. Nawaz, E6-associated protein (E6-AP) is a dual function coactivator of steroid hormone receptors. Nucl Recept Signal 6, e006 (2008).
50. Anderson M P, Mochizuki T, Xie J, Fischler W, Manger J P, Talley E M, Scammell T E, Tonegawa S. Thalamic Cav3.1 T-type calcium channel plays a crucial role in stabilizing sleep. Proc Natl Acad Sci USA. 2005; 102(5):1743-1748.
51. Crawley J N. Mouse behavioral assays relevant to the symptoms of autism. Brain Pathol. 2007; 17(4):448-459.
52. Greer P L, Hanayama R, Bloodgood B L, Mardinly A R, Lipton D M, Flavell S W, Kim T K, Griffith E C, Waldon Z, Maehr R, Ploegh H L, Chowdhury S, Worley P F, Steen J, Greenberg M E. The Angelman Syndrome protein Ube3A regulates synapse development by ubiquitinating arc. Cell. 2010; 140(5):704-716.
53. Gustin R M, Bichell T J, Bubser M, Daily J, Filonova I, Mrelashvili D, Deutch A Y, Colbran R J, Weeber E J, Haas K F. Tissue-specific variation of Ube3a protein expression in rodents and in a mouse model of Angelman syndrome. Neurobiol Dis. 2010; 39(3):283-91.
54. Scattoni M L, McFarlane H G, Zhodzishsky V, Caldwel H K, Young W S, Ricceri L and Crawley J N. Reduced ultrasonic vocalizations in vasopressin 1b knockout mice. Behav Brain Res 2008; 187(2):371-378.
55. Smith S E, Li J, Garbett K, Mimics K, Patterson P H. Maternal immune activation alters fetal brain development through interleukin-6. J Neurosci. 2007; 27(40):10695-10702.
56. Zhou Y-D, Lee S, Jin Z, Wright M, Smith S E P, Anderson M P. Arrested maturation of excitatory synapses in autosomal dominant lateral temporal lobe epilepsy. Nature Medicine. 2009; 15(10):1208-1214.

All publications, patents, patent applications, websites, and database entries (e.g., sequence database entries) mentioned herein, including those items listed below, are hereby incorporated by reference in their entirety for the relevant teachings contained therein, as if each individual publication or patent was specifically and individually indicated to be incorporated by reference. In the case where the present specification and a document incorporated by reference include conflicting disclosure, the present specification shall control.

### SCOPE AND EQUIVALENTS

While several embodiments of the present invention have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present invention. More generally, those skilled in the art will readily appreciate that all methods, method steps, compounds, compositions, parameters, dimensions, materials, and configurations described herein are meant to be exemplary and that the actual parameters, dimensions, materials, and/or configurations will depend upon the specific application or applications for which the teachings of the present invention is/are used. Those skilled in the art will recognize, or be able to ascertain using no more than routine experimentation, many equivalents to the specific embodiments of the invention described herein. It is, therefore, to be understood that the foregoing embodiments are presented by way of example only and that, within the scope of the appended claims and equivalents thereto, the invention may be practiced otherwise than as specifically described and claimed. The present invention is directed to each individual method, method step, compound, composition, feature, system, article, material, and/or kit described herein. In addition, any combination of two or more such methods, method steps, compounds, compositions, features, systems, articles, materials, and/or kits, if not mutually inconsistent, is included within the scope of the present invention.

The terms and expressions that have been employed are used as terms of description and not of limitation, and there is no intention in the use of such terms and expressions of excluding any equivalents of the features described, it being recognized that various modifications are possible within the scope of the invention. All definitions, as defined and used herein, should be understood to control over dictionary definitions, definitions in documents incorporated by reference, and/or ordinary meanings of the defined terms.

The indefinite articles “a” and “an”, as used herein in the specification and in the claims, unless clearly indicated to the contrary, should be understood to mean “at least one.” The phrase “and/or,” as used herein in the specification and in the claims, should be understood to mean “either or both” of the elements so conjoined, i.e., elements that are conjunctively present in some cases and disjunctively present in other cases. Other elements may optionally be present other than the elements specifically identified by the “and/or” clause, whether related or unrelated to those elements specifically identified unless clearly indicated to the contrary. Thus, as a non-limiting example, a reference to “A and/or B”, when used in conjunction with open-ended language such as “comprising” can refer, in one embodiment, to A without B (optionally including elements other than B); in another embodiment, to B without A (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of”, when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified: within the list of elements to which the phrase “at least one” refers, whether related or unrelated to those elements specifically identified. Thus, as a non-limiting example, “at least one of A and B” (or, equivalently, “at least one of A or B,” or, equivalently, “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the acts of the method are recited.

1. An isolated transgenic mammalian cell comprising
one or more isolated nucleic acid sequence(s) encoding a ubiquitin ligase 3a (ube3a) protein; or
one or more exogenous nucleic acid sequence(s) encoding a ube3a protein;
one or more recombinant nucleic acid sequence(s) encoding a ube3a protein; or
one or more nucleic acid sequence(s) encoding a ube3a protein in addition to any endogenous copies of nucleic acid sequences encoding a ube3a protein.

2. The transgenic mammalian cell of claim 1, wherein the nucleic acid sequence(s) encoding a ube3a protein are stably integrated into the genome of the cell.

3-6. (canceled)

7. The transgenic mammalian cell of claim 1, wherein the genome of the transgenic mammal comprises three endogenous nucleic acid sequences encoding a ube3a protein, optionally wherein the genome of the transgenic mammal comprises an idic15 mutation.

8. The transgenic mammalian cell of claim 1, wherein the cell is a human cell, a non-human mammalian cell, or a mouse cell.

9. The transgenic mammalian cell of claim 7, wherein the mouse cell is derived from a mouse of FVB, dup15 or idic15 genetic background.

10-11. (canceled)

12. The transgenic mammalian cell of claim 1, wherein the one or more isolated nucleic acid sequence(s) encoding a ube3a protein comprise a ube3a cDNA.

13-16. (canceled)

17. The transgenic mammalian cell of claim 1, wherein the one or more isolated nucleic acid sequence(s) encoding a ube3a protein comprise a fragment of mouse chromosome 7.

18-20. (canceled)

21. The transgenic mammalian cell of claim 17, wherein the fragment comprises at least about 1 kb, at least about 2 kb, at least about 3 kb, at least about 4 kb, at least about 5 kb, at least about 10 kb, at least about 20 kb, at least about 25 kb, at least about 30 kb, at least about 40 kb, at least about 50 kb, at least about 60 kb, at least about 70 kb, at least about 80 kb, at least about 90 kb, or at least about 100 kb of the chromosome 7 region immediately upstream (5′) of the exon-intron coding sequence of ube3a.

22. (canceled)

23. The transgenic mammalian cell of claim 17, wherein the fragment comprises at least about 1 kb, at least about 2 kb, at least about 3 kb, at least about 4 kb, at least about 5 kb, at least about 10 kb, at least about 20 kb, at least about 25 kb, at least about 30 kb, at least about 40 kb, at least about 50 kb, at least about 60 kb, at least about 70 kb, at least about 80 kb, at least about 90 kb, or at least about 100 kb of the chromosome 7 region immediately downstream (3′) of the exon-intron coding sequence of ube3a.

24. (canceled)

25. The transgenic mammalian cell of claim 1, wherein the one or more isolated nucleic acid sequence(s) encoding a ube3a protein further comprises a sequence encoding a tag.

26-38. (canceled)

39. The transgenic mammalian cell of claim 1, wherein the cell is comprised in a non-human mammal.

40. A non-human mammal comprising at least one cell of claim 1.

41. A non-human mammal comprising at least one germ cell according to claim 1.

42-43. (canceled)

44. The non-human mammal of claim 40, wherein the non-human mammal is a mouse.

45. The mouse of claim 44, wherein the mouse exhibits one or more of (i) impaired social interaction; (ii) defective communication; and/or (iii) repetitive behavior.

46. A non-human mammal comprising at least one expression construct comprising a nucleic acid sequence encoding a ube3a protein stably integrated into the genome of at least one cell comprised in the non-human mammal.

47. (canceled)

48. A method of identifying an agent for the treatment of a symptom associated with autism, the method comprising,
(i) administering a candidate agent to a transgenic non-human mammal comprising an isolated, exogenous, or additional ube3a protein-encoding nucleic acid sequence or expressing an elevated level of ube3a protein, and exhibiting or expected to develop at least one symptom associated with autism;
(ii) determining whether the administration of the candidate agent effected an amelioration of the symptom, wherein
if the administration of the candidate agent effected an amelioration of the symptom, then the candidate agent is identified as an agent for the treatment of a symptom associated with autism.

49. (canceled)

50. A method of identifying an agent for the treatment of a pathological characteristic associated with autism, the method comprising,
(i) contacting a candidate agent with a transgenic cell comprising an isolated, exogenous, or additional ube3a protein-encoding nucleic acid sequence or expressing an elevated level of ube3a protein, and exhibiting or expected to develop at least one pathological characteristic associated with autism;
(ii) determining whether the candidate agent effected an amelioration of the pathological characteristic in the cell, wherein
if an amelioration of the pathological characteristic is observed as a result of the contacting, then the candidate agent is identified as an agent for the treatment of a pathological characteristic associated with autism.

51. (canceled)

52. A method of identifying a diagnostic marker for autism, the method comprising
assessing the expression level of a biomolecule in a cell, tissue, or sample of a transgenic non-human mammal comprising an increased ube3a protein-encoding nucleic acids copy number and comparing the expression level to a control or reference level, wherein
if the biomolecule expression level in the transgenic mammal is different from the control level, then differential expression of the biomolecule is identified as a diagnostic biomarker for autism.

53-54. (canceled)

55. A method of diagnosing an increased risk of developing autism or an autism spectrum disorder in a subject, the method comprising
determining a level of a ube3a protein in a sample obtained from the subject and comparing the level of ube3a determined in the subject to a control or reference level, wherein
if the level of ube3a protein detected in the subject is higher than the control or reference level, the subject is identified as a subject at an increased risk of developing autism or an autism spectrum disorder.

56-57. (canceled)


SEQUENCE LISTING (14 sequences)

SEQ ID NO: 1 - DNA, 3888 bp, Mus musculus
ctgtcgggatactcggtccgcccacctagtcctctcgtccagtgctgcgttcgcgagatccgtatttctcccaagatggtggcgctcctctttgggtgactccaggagacgacagggcctttcgtctttgccagcacctcgtcgcccctcctgcgctcgctctctcgctcgcgcaccgggccacgcagctgttcaccgcctcgttacgcttctcttccgtcgacctgtcgctgacggtggcgcctccttctgcttctcttcggagttgctcgccgccctcgccccccactgtggacagatcgcgacagcagcgcttcagcgccgacttcaaggttgcccaggcgcctggcctctcggcctcggtttcctgaggagaagcgcgggtcccgcatgagacccggcggtggcgccagcgaaagggaacgaggcggtggcgggcggcggcggtggacgagggcgacaaggaccagtgaggcggccgcagctgcgagggccgcagcccacgcgcgggggcgaggacagatcaccaggagaatcccagtctgaggacattgaagctagccgaatgaagcgagcagctgcaaagcatctaatagaacgctactaccatcagttaactgagggctgtggaaatgaggcctgcacgaatgagttttgtgcttcctgtccaacttttcttcgtatggataacaatgcagcagctattaaagcccttgagctttataaaattaatgcaaaactctgtgatcctcatccctccaagaaaggagcaagctcagcttaccttgagaactcaaaaggtgcatctaacaactcagagataaaaatgaacaagaaggaaggaaaagattttaaagatgtgatttacctaactgaagagaaagtatatgaaatttatgaattttgtagagagagtgaggattattcccctttaattcgtgtaattggaagaatattttctagtgctgaggcactggttctgagctttcggaaagtcaaacagcacacaaaggaggaattgaaatctcttcaagaaaaggatgaagacaaggatgaagatgaaaaggaaaaagctgcatgttctgctgctgctatggaagaagactcagaagcatcttcttcaaggatgggtgatagttcacagggagacaacaatgtacaaaaattaggtcctgatgatgtgactgtggatattgatgctattagaagggtctacagcagtttgctcgctaatgaaaaattagaaactgccttcctgaatgcacttgtatatctgtcacctaacgtggaatgtgatttgacatatcataatgtgtatactcgagatcctaattatctcaatttgttcattattgtaatggagaatagtaatctccacagtcctgaatatctggaaatggcgttgccattattttgcaaagctatgtgtaagctaccccttgaagctcaaggaaaactgattaggctgtggtctaaatacagtgctgaccagattcggagaatgatggaaacatttcagcaacttattacctacaaagtcataagcaatgaatttaatagccgaaatctagtgaatgatgatgatgccattgttgctgcttcaaagtgtttgaaaatggtttactatgcaaatgtagtgggaggggatgtggacacaaatcataatgaggaagatgatgaagaacccatacctgagtccagcgaattaacacttcaggagcttctgggagatgaaagaagaaataagaaaggtcctcgagtggatccactagaaaccgaacttggcgttaaaactctagactgtcgaaaaccacttatctcctttgaagaattcattaatgaaccactgaatgatgttctagaaatggacaaagattatacctttttcaaagttgaaacagagaacaaattctcttttatgacatgtccctttatattgaatgctgtcacaaagaatctgggattatattatgacaatagaattcgcatgtacagtgaaagaagaatcactgttctttacagcctagttcaaggacagcagttgaatccgtatttgagactcaaagtcagacgtgaccatattatagatgatgcactggtccggctagagatgattgctatggaaaatcctgcagacttgaagaagcagttgtatgtggaatttgaaggagaacaaggagtagatgagggaggcgtttccaaagagttttttcagttggttgtggaggaaatttttaatccagatattggtatgttcacatatgatgaagctacgaaattattttggtttaatccatcttcttttgaaactgagggtcagtttactctgattggcatagtcctgggtctggctatttacaataattgtatactggatgtccattttcccatggttgtatacaggaagctaatggggaaaaaaggaacctttcgtgacttgggagactctcacccagttttatatcagagtttaaaggatttattggaatatgaagggagtgtggaagatgatatgatgatcactttccagatatcacagacagatctttttggtaacccaatgatgtatgatctaaaagaaaatggtgataaaattccaattacaaatgaaaacaggaaggaatttgtcaatctctattcagactacattctcaataaatctgtagaaaaacaattcaaggcatttcgcagaggttttcatatggtgactaatgaatcgcccttaaaatacttattcagaccagaagaaattgaattgcttatatgtggaagccggaatctagatttccaggcactagaagaaactacagagtatgacggtggctatacgagggaatctgttgtgattaggtaaggtgtttaattcttaaaaaggaagattttattcatcaaacatgtagatgtgtgcttttgtgtcctgtatctgtaggtactggttaccaaacaagtaagctcaaaaatagacctgtattaatatttccaattttcatgcagtctaatgctttatttcatgaattaaatgatttaagtctcatattttctcaaccctttgccttatttttggtcatgtgtaagatggcacattatttagtctttaagatacttgggaagaaccatgtatactagtgattctgaacaattcttaggacagtattaccactaacatcgttctctagtcaaatgcccttatttctacttctgtaatatgctactatccaattctgaaagatctttccccccatcttctaatgtgactgatcaaaatgcagagtagtctttttggcatccactatgatgtcataggtatttaaacagttatctttttgtagatcacttgagctataagactcaaatatgttaacaatagaatgaatattaactgtgtctagtaatgatacattatcattgttatatttatattacagtattactttattcatttaagtttgtagaagattactcttgctttgcccttttttttttaatagaaaagcaaatatgttatttattcagcttttaggtaattaaataacaaaattcagagtaaagcaaaacaaaaaccataacatgtcatatgatatatcatttctaagcacaatggcaattattaatgaatataaaaatttatcattcatatttgcttctaacaccagtcacaaaagtggcaaccattatattgctgctcagttttaaaggtaattcataacagggataaacatggtaatacagaagccttaatgggaatatcctagtattatctctacaatatggcaaaataatgttttagattgattatgattaatgtatgcattttgattattatccttttgttattggcaatagaattatcatgacagtggggctgttacaaataaagttttcattctt

SEQ ID NO: 2 - DNA, 4910 bp, Mus musculus
gtcgggatactcggtccgcccacctagtcctctcgtccagtgctgcgttcgcgagatccgtatttctcccaagatggtggcgctcctctttgggtgactccaggagacgacagggcctttcgtctttgccagcacctcgtcgcccctcctgcgctcgctctctcgctcgcgcaccgggccacgcagctgttcaccgcctcgttacgcttctcttccgtcgacctgtcgctgacggtggcgcctccttctgcttctcttcggagttgctcgccgccctcgccccccactgtggacagatcgcgacagcagcgcttcagcgccgacttcaaggttgcccaggcgcctggcctctcggcctcggtttcctgaggagaagcgcgggtcccgcatgagacccggcggtggcgccagcgaaagggaacgaggcggtggcgggcggcggcggtggacgagggcgacaaggaccagtgaggcggccgcagctgcgagggccgcagcccacgcgcgggggcgaggacagatcaccaggagaatcccagtctgaggacattgaagctagccgaatgaagcgagcagctgcaaagcatctaatagaacgctactaccatcagttaactgagggctgtggaaatgaggcctgcacgaatgagttttgtgcttcctgtccaacttttcttcgtatggataacaatgcagcagctattaaagcccttgagctttataaaattaatgcaaaactctgtgatcctcatccctccaagaaaggagcaagctcagcttaccttgagaactcaaaaggtgcatctaacaactcagagataaaaatgaacaagaaggaaggaaaagattttaaagatgtgatttacctaactgaagagaaagtatatgaaatttatgaattttgtagagagagtgaggattattcccctttaattcgtgtaattggaagaatattttctagtgctgaggcactggttctgagctttcggaaagtcaaacagcacacaaaggaggaattgaaatctcttcaagaaaaggatgaagacaaggatgaagatgaaaaggaaaaagctgcatgttctgctgctgctatggaagaagactcagaagcatcttcttcaaggatgggtgatagttcacagggagacaacaatgtacaaaaattaggtcctgatgatgtgactgtggatattgatgctattagaagggtctacagcagtttgctcgctaatgaaaaattagaaactgccttcctgaatgcacttgtatatctgtcacctaacgtggaatgtgatttgacatatcataatgtgtatactcgagatcctaattatctcaatttgttcattattgtaatggagaatagtaatctccacagtcctgaatatctggaaatggcgttgccattattttgcaaagctatgtgtaagctaccccttgaagctcaaggaaaactgattaggctgtggtctaaatacagtgctgaccagattcggagaatgatggaaacatttcagcaacttattacctacaaagtcataagcaatgaatttaatagccgaaatctagtgaatgatgatgatgccattgttgctgcttcaaagtgtttgaaaatggtttactatgcaaatgtagtgggaggggatgtggacacaaatcataatgaggaagatgatgaagaacccatacctgagtccagcgaattaacacttcaggagcttctgggagatgaaagaagaaataagaaaggtcctcgagtggatccactagaaaccgaacttggcgttaaaactctagactgtcgaaaaccacttatctcctttgaagaattcattaatgaaccactgaatgatgttctagaaatggacaaagattatacctttttcaaagttgaaacagagaacaaattctcttttatgacatgtccctttatattgaatgctgtcacaaagaatctgggattatattatgacaatagaattcgcatgtacagtgaaagaagaatcactgttctttacagcctagttcaaggacagcagttgaatccgtatttgagactcaaagtcagacgtgaccatattatagatgatgcactggtccggctagagatgattgctatggaaaatcctgcagacttgaagaagcagttgtatgtggaatttgaaggagaacaaggagtagatgagggaggcgtttccaaagagttttttcagttggttgtggaggaaatttttaatccagatattggtatgttcacatatgatgaagctacgaaattattttggtttaatccatcttcttttgaaactgagggtcagtttactctgattggcatagtcctgggtctggctatttacaataattgtatactggatgtccattttcccatggttgtatacaggaagctaatggggaaaaaaggaacctttcgtgacttgggagactctcacccagttttatatcagagtttaaaggatttattggaatatgaagggagtgtggaagatgatatgatgatcactttccagatatcacagacagatctttttggtaacccaatgatgtatgatctaaaagaaaatggtgataaaattccaattacaaatgaaaacaggaaggaatttgtcaatctctattcagactacattctcaataaatctgtagaaaaacaattcaaggcatttcgcagaggttttcatatggtgactaatgaatcgcccttaaaatacttattcagaccagaagaaattgaattgcttatatgtggaagccggaatctagatttccaggcactagaagaaactacagagtatgacggtggctatacgagggaatctgttgtgattagggagttctgggaaattgttcattcgtttacagatgaacagaaaagactctttctgcagtttacaacaggcacagacagagcacctgttggaggactaggaaaattgaagatgattatagccaaaaatggcccagacacagaaaggttacctacatctcatacttgctttaatgtccttttacttccggaatattcaagcaaagaaaaacttaaagagagattgttgaaggccatcacatatgccaaaggatttggcatgctgtaaacaaaaagaaaaagaaaaagaaaaagaaaaagttaaaaaataaatataagagggataatttgatggtaatagtatcccagtacaaaaaggctgtaagatagtgaaccacagtagtcatctatgtctgtgcctcccttcttcattggggacattgtgggctggaacagcagatttcagctgcatatatgaacaaatcctttattattattataattatttttttgcgtgaaagtgttacatattctttcacttgtatgtacagagaggttttctgaatatttattttaagggttaaatcacttttgcttgtgtttattactgcttgaggttgagcctttttgagtatttaagatatatataccaacgaaactattctcgcaaggaaaacattgccaccatttgtagaacatgtaatcttcaagtatgtgctattttttgtccctgtatctaagtcaaatcaggaacttttttctaacaatttgcttttgaaacttgaagtcaaggaaacagtgtggtgcaagtactgctgttctagcccccaaagagttttctgtacaaaattttgagaaccaataaagatggaagggagaacttggaatgtttgaaccacagccctcagaactttagtaacagcacaacaaattaaaacaactcatgccacagtatgttgtcttcatgtgtcttgcaatgaactgtttcagtagccaatcctcttagtatatgaaaggacagggattttttttttgtttttgttgttgttgttgttgttgttgtttttgttgttgttgttgtttttgttgtttaagtttactggggaaagtgcatctggccaaatgataggatagtcaagcctattgcaacaaaattaggaagtttgttgtataaataagcatgtaaaagtgcacttaaaatgaatctttattattgctgagattttaatagacaatccaaagtctccccttctgttgccgtcatcttgtttaatcaaccatttttcaaggcactcgatcagtgttgcagcataacagaaagtacagctactgtgccttgtgttacttatttacacagttagcaggcctggaaatgaatggaactagtactcctgagaaataaattgtatatcccccaaattaaaatttacttcaaaggtgttaaagatttcatgtcctatattaaagtacaaataggcttaaattactggatatttaatgtagtttcccatccctagtcttctatgtctgtgatgttaatttcttttgttgcataacaaaataaaagaattatgtatttttaactaaggagagacatactggtatatcattttactacaagctacagataacctgttgagcttgtgccttgattgttttaacaactagtgcaaatcaacctgatgattttaattggcaggggataatggtagctttcaaatcattggaaggggaaaaggatgtcttaggattattttctttcttgtagtagttgagacagagctcttatttactgtaatgctaaatgaaacagtggcttaaatattttaatgggaaaagagaacacagtgcgttccatattgtgataaggtaacgtgaggtttttttgttttgttttgttttcttttttttttttctgagctagcctttagaacactgttgtggtatgtatgctaccttgattataggaccccctaaatgtgactatagtcatcttaatgggcatcttgtccactgtgcttcttatgtattatgaaagtgataagaagacaaattaagtgggtatattttataaaataaattcatg

SEQ ID NO: 3 - DNA, 5097 bp, Mus musculus
gtcgggatactcggtccgcccacctagtcctctcgtccagtgctgcgttcgcgagatccgtatttctcccaagatggtggcgctcctctttgggtgactccaggagacgacagggcctttcgtctttgccagcacctcgtcgcccctcctgcgctcgctctctcgctcgcgcaccgggccacgcagctgttcaccgcctcgttacgcttctcttccgtcgacctgtcgctgacggtggcgcctccttctgcttctcttcggagttgctcgccgccctcgccccccactgtggacagatcgcgacagcagcgcttcagcgccgacttcaaggttgcccaggcgcctggcctctcggcctcggtttcctgaggagaagcgcgggtcccgcatgagacccggcggtggcgccagcgaaagggaacgaggcggtggcgggcggcggcggtggacgagggcgacaaggaccagtgaggcggccgcagctgcgagggccgcagcccacgcgcgggggcgaggacaggttaaaaaatctctctaagagcctgattttagagttcaccagctcctcagaagtttggcgaaatatgaattattaagcctacgttcagatcaagttagcagctagactggtgtgacaacctgtttttaatcagtgactcaaagctgttatcaccctgatgtcaccgaatggccacagcttgtaaaagatcaccaggagaatcccagtctgaggacattgaagctagccgaatgaagcgagcagctgcaaagcatctaatagaacgctactaccatcagttaactgagggctgtggaaatgaggcctgcacgaatgagttttgtgcttcctgtccaacttttcttcgtatggataacaatgcagcagctattaaagcccttgagctttataaaattaatgcaaaactctgtgatcctcatccctccaagaaaggagcaagctcagcttaccttgagaactcaaaaggtgcatctaacaactcagagataaaaatgaacaagaaggaaggaaaagattttaaagatgtgatttacctaactgaagagaaagtatatgaaatttatgaattttgtagagagagtgaggattattcccctttaattcgtgtaattggaagaatattttctagtgctgaggcactggttctgagctttcggaaagtcaaacagcacacaaaggaggaattgaaatctcttcaagaaaaggatgaagacaaggatgaagatgaaaaggaaaaagctgcatgttctgctgctgctatggaagaagactcagaagcatcttcttcaaggatgggtgatagttcacagggagacaacaatgtacaaaaattaggtcctgatgatgtgactgtggatattgatgctattagaagggtctacagcagtttgctcgctaatgaaaaattagaaactgccttcctgaatgcacttgtatatctgtcacctaacgtggaatgtgatttgacatatcataatgtgtatactcgagatcctaattatctcaatttgttcattattgtaatggagaatagtaatctccacagtcctgaatatctggaaatggcgttgccattattttgcaaagctatgtgtaagctaccccttgaagctcaaggaaaactgattaggctgtggtctaaatacagtgctgaccagattcggagaatgatggaaacatttcagcaacttattacctacaaagtcataagcaatgaatttaatagccgaaatctagtgaatgatgatgatgccattgttgctgcttcaaagtgtttgaaaatggtttactatgcaaatgtagtgggaggggatgtggacacaaatcataatgaggaagatgatgaagaacccatacctgagtccagcgaattaacacttcaggagcttctgggagatgaaagaagaaataagaaaggtcctcgagtggatccactagaaaccgaacttggcgttaaaactctagactgtcgaaaaccacttatctcctttgaagaattcattaatgaaccactgaatgatgttctagaaatggacaaagattatacctttttcaaagttgaaacagagaacaaattctcttttatgacatgtccctttatattgaatgctgtcacaaagaatctgggattatattatgacaatagaattcgcatgtacagtgaaagaagaatcactgttctttacagcctagttcaaggacagcagttgaatccgtatttgagactcaaagtcagacgtgaccatattatagatgatgcactggtccggctagagatgattgctatggaaaatcctgcagacttgaagaagcagttgtatgtggaatttgaaggagaacaaggagtagatgagggaggcgtttccaaagagttttttcagttggttgtggaggaaatttttaatccagatattggtatgttcacatatgatgaagctacgaaattattttggtttaatccatcttcttttgaaactgagggtcagtttactctgattggcatagtcctgggtctggctatttacaataattgtatactggatgtccattttcccatggttgtatacaggaagctaatggggaaaaaaggaacctttcgtgacttgggagactctcacccagttttatatcagagtttaaaggatttattggaatatgaagggagtgtggaagatgatatgatgatcactttccagatatcacagacagatctttttggtaacccaatgatgtatgatctaaaagaaaatggtgataaaattccaattacaaatgaaaacaggaaggaatttgtcaatctctattcagactacattctcaataaatctgtagaaaaacaattcaaggcatttcgcagaggttttcatatggtgactaatgaatcgcccttaaaatacttattcagaccagaagaaattgaattgcttatatgtggaagccggaatctagatttccaggcactagaagaaactacagagtatgacggtggctatacgagggaatctgttgtgattagggagttctgggaaattgttcattcgtttacagatgaacagaaaagactctttctgcagtttacaacaggcacagacagagcacctgttggaggactaggaaaattgaagatgattatagccaaaaatggcccagacacagaaaggttacctacatctcatacttgctttaatgtccttttacttccggaatattcaagcaaagaaaaacttaaagagagattgttgaaggccatcacatatgccaaaggatttggcatgctgtaaacaaaaagaaaaagaaaaagaaaaagaaaaagttaaaaaataaatataagagggataatttgatggtaatagtatcccagtacaaaaaggctgtaagatagtgaaccacagtagtcatctatgtctgtgcctcccttcttcattggggacattgtgggctggaacagcagatttcagctgcatatatgaacaaatcctttattattattataattatttttttgcgtgaaagtgttacatattctttcacttgtatgtacagagaggttttctgaatatttattttaagggttaaatcacttttgcttgtgtttattactgcttgaggttgagcctttttgagtatttaagatatatataccaacgaaactattctcgcaaggaaaacattgccaccatttgtagaacatgtaatcttcaagtatgtgctattttttgtccctgtatctaagtcaaatcaggaacttttttctaacaatttgcttttgaaacttgaagtcaaggaaacagtgtggtgcaagtactgctgttctagcccccaaagagttttctgtacaaaattttgagaaccaataaagatggaagggagaacttggaatgtttgaaccacagccctcagaactttagtaacagcacaacaaattaaaacaactcatgccacagtatgttgtcttcatgtgtcttgcaatgaactgtttcagtagccaatcctcttagtatatgaaaggacagggattttttttttgtttttgttgttgttgttgttgttgttgtttttgttgttgttgttgtttttgttgtttaagtttactggggaaagtgcatctggccaaatgataggatagtcaagcctattgcaacaaaattaggaagtttgttgtataaataagcatgtaaaagtgcacttaaaatgaatctttattattgctgagattttaatagacaatccaaagtctccccttctgttgccgtcatcttgtttaatcaaccatttttcaaggcactcgatcagtgttgcagcataacagaaagtacagctactgtgccttgtgttacttatttacacagttagcaggcctggaaatgaatggaactagtactcctgagaaataaattgtatatcccccaaattaaaatttacttcaaaggtgttaaagatttcatgtcctatattaaagtacaaataggcttaaattactggatatttaatgtagtttcccatccctagtcttctatgtctgtgatgttaatttcttttgttgcataacaaaataaaagaattatgtatttttaactaaggagagacatactggtatatcattttactacaagctacagataacctgttgagcttgtgccttgattgttttaacaactagtgcaaatcaacctgatgattttaattggcaggggataatggtagctttcaaatcattggaaggggaaaaggatgtcttaggattattttctttcttgtagtagttgagacagagctcttatttactgtaatgctaaatgaaacagtggcttaaatattttaatgggaaaagagaacacagtgcgttccatattgtgataaggtaacgtgaggtttttttgttttgttttgttttcttttttttttttctgagctagcctttagaacactgttgtggtatgtatgctaccttgattataggaccccctaaatgtgactatagtcatcttaatgggcatcttgtccactgtgcttcttatgtattatgaaagtgataagaagacaaattaagtgggtatattttataaaataaattcatg

SEQ ID NO: 4 - DNA, 5276 bp, Homo sapiens
agccagtcctcccgtcttgcgccgcggccgcgagatccgtgtgtctcccaagatggtggcgctgggctcggggtgactacaggagacgacggggccttttcccttcgccaggacccgacacaccaggcttcgctcgctcgcgcacccctccgccgcgtagccatccgccagcgcgggcgcccgccatccgccgcctacttacgcttcacctctgccgacccggcgcgctcggctgcgggcggcggcgcctccttcggctcctcctcggaatagctcgcggcctgtagcccctggcaggagggcccctcagccccccggtgtggacaggcagcggcggctggcgacgaacgccgggatttcggcggccccggcgctccctttcccggcctcgttttccggataaggaagcgcgggtcccgcatgagccccggcggtggcggcagcgaaagagaacgaggcggtggcgggcggaggcggcgggcgagggcgactacgaccagtgaggcggccgccgcagcccaggcgcgggggcgacgacaggttaaaaatctgtaagagcctgattttagaattcaccagctcctcagaagtttggcgaaatatgagttattaagcctacgctcagatcaaggtagcagctagactggtgtgacaacctgtttttaatcagtgactcaaagctgtgatcaccctgatgtcaccgaatggccacagcttgtaaaagagagttacagtggaggtaaaaggagtggcttgcaggatggagaagctgcaccagtgttattggaaatcaggagaacctcagtctgacgacattgaagctagccgaatgaagcgagcagctgcaaagcatctaatagaacgctactaccaccagttaactgagggctgtggaaatgaagcctgcacgaatgagttttgtgcttcctgtccaacttttcttcgtatggataataatgcagcagctattaaagccctcgagctttataagattaatgcaaaactctgtgatcctcatccctccaagaaaggagcaagctcagcttaccttgagaactcgaaaggtgcccccaacaactcctgctctgagataaaaatgaacaagaaaggcgctagaattgattttaaagatgtgacttacttaacagaagagaaggtatatgaaattcttgaattatgtagagaaagagaggattattcccctttaatccgtgttattggaagagttttttctagtgctgaggcattggtacagagcttccggaaagttaaacaacacaccaaggaagaactgaaatctcttcaagcaaaagatgaagacaaagatgaagatgaaaaggaaaaagctgcatgttctgctgctgctatggaagaagactcagaagcatcttcctcaaggataggtgatagctcacagggagacaacaatttgcaaaaattaggccctgatgatgtgtctgtggatattgatgccattagaagggtctacaccagattgctctctaatgaaaaaattgaaactgcctttctcaatgcacttgtatatttgtcacctaacgtggaatgtgacttgacgtatcacaatgtatactctcgagatcctaattatctgaatttgttcattatcgtaatggagaatagaaatctccacagtcctgaatatctggaaatggctttgccattattttgcaaagcgatgagcaagctaccccttgcagcccaaggaaaactgatcagactgtggtctaaatacaatgcagaccagattcggagaatgatggagacatttcagcaacttattacttataaagtcataagcaatgaatttaacagtcgaaatctagtgaatgatgatgatgccattgttgctgcttcgaagtgcttgaaaatggtttactatgcaaatgtagtgggaggggaagtggacacaaatcacaatgaagaagatgatgaagagcccatccctgagtccagcgagctgacacttcaggaacttttgggagaagaaagaagaaacaagaaaggtcctcgagtggaccccctggaaactgaacttggtgttaaaaccctggattgtcgaaaaccacttatcccttttgaagagtttattaatgaaccactgaatgaggttctagaaatggataaagattatacttttttcaaagtagaaacagagaacaaattctcttttatgacatgtccctttatattgaatgctgtcacaaagaatttgggattatattatgacaatagaattcgcatgtacagtgaacgaagaatcactgttctctacagcttagttcaaggacagcagttgaatccatatttgagactcaaagttagacgtgaccatatcatagatgatgcacttgtccggctagagatgatcgctatggaaaatcctgcagacttgaagaagcagttgtatgtggaatttgaaggagaacaaggagttgatgagggaggtgtttccaaagaattttttcagctggttgtggaggaaatcttcaatccagatattggtatgttcacatacgatgaatctacaaaattgttttggtttaatccatcttcttttgaaactgagggtcagtttactctgattggcatagtactgggtctggctatttacaataactgtatactggatgtacattttcccatggttgtctacaggaagctaatggggaaaaaaggaacttttcgtgacttgggagactctcacccagttctatatcagagtttaaaagatttattggagtatgaagggaatgtggaagatgacatgatgatcactttccagatatcacagacagatctttttggtaacccaatgatgtatgatctaaaggaaaatggtgataaaattccaattacaaatgaaaacaggaaggaatttgtcaatctttattctgactacattctcaataaatcagtagaaaaacagttcaaggcttttcggagaggttttcatatggtgaccaatgaatctcccttaaagtacttattcagaccagaagaaattgaattgcttatatgtggaagccggaatctagatttccaagcactagaagaaactacagaatatgacggtggctataccagggactctgttctgattagggagttctgggaaatcgttcattcatttacagatgaacagaaaagactcttcttgcagtttacaacgggcacagacagagcacctgtgggaggactaggaaaattaaagatgattatagccaaaaatggcccagacacagaaaggttacctacatctcatacttgctttaatgtgcttttacttccggaatactcaagcaaagaaaaacttaaagagagattgttgaaggccatcacgtatgccaaaggatttggcatgctgtaaaacaaaacaaaacaaaataaaacaaaaaaaaggaaggaaaaaaaaagaaaaaatttaaaaaattttaaaaatataacgagggataaatttttggtggtgatagtgtcccagtacaaaaaggctgtaagatagtcaaccacagtagtcacctatgtctgtgcctcccttctttattggggacatgtgggctggaacagcagatttcagctacatatatgaacaaatcctttattattattataattatttttttgcgtgaaagtgttacatattctttcacttgtatgtacagagaggtttttctgaatatttattttaagggttaaatcacttttgcttgtgtttattactgcttgaggttgagccttttgagtatttaaaaaatatataccaacagaactactctcccaaggaaaatattgccaccatttgtagaccacgtaaccttcaagtatgtgctacttttttgtccctgtatctaactcaaatcaggaactgtattttttttaatgatttgcttttgaaacttgaagtcttgaaaacagtgtgatgcaattactgctgttctagcccccaaagagttttctgtgcaaaatcttgagaatcaatcaataaagaaagatggaaggaagggagaaattggaatgttttaactgcagccctcagaactttagtaacagcacaacaaattaaaaacaaaaacaactcatgccacagtatgtcgtcttcatgtgtcttgcaatgaactgtttcagtagccaatcctctttcttagtatatgaaaggacagggatttttgttcttgttgttctcgttgttgttttaagtttactggggaaagtgcatttggccaaatgaaatggtagtcaagcctattgcaacaaagttaggaagtttgttgtttgtttattataaacaaaaagcatgtgaaagtgcacttaagatagagtttttattaattacttacttattacctagattttaaatagacaatccaaagtctccccttcgtgttgccatcatcttgttgaatcagccattttatcgaggcacgtgatcagtgttgcaacataatgaaaaagatggctactgtgccttgtgttacttaatcatacagtaagctgacctggaaatgaatgaaactattactcctaagaattacattgtatagccccacagattaaatttaattaattaattcaaaacatgttaaacgttactttcatgtactatggaaaagtacaagtaggtttacattactgatttccagaagtaagtagtttcccctttcctagtcttctgtgtatgtgatgttgttaatttcttttattgcattataaaataaaaggattatgtatttttaactaaggtgagacattgatatatccttttgctacaagctatagctaatgtgctgagcttgtgccttggtgattgattgattgattgactgattgttttaactgattactgtagatcaacctgatgatttgtttgtttgaaattggcaggaaaaatgcagctttcaaatcattggggggagaaaaaggatgtctttcaggattattttaattaatttttttcataattgagacagaactgtttgttatgtaccataatgctaaataaaactgtggcacttttcaccataatttaatttagtggaaaaagaagacaatgctttccatattgtgataaggtaacatggggtttttctgggccagcctttagaacactgttagggtacatacgctaccttgatgaaagggaccttcgtgcaactgtagtcatcttaaaggcttctcatccactgtgcttcttaatgtgtaattaaagtgaggagaaattaaatactctgagggcgttttatataataaattcgtgaaga

SEQ ID NO: 5 - DNA, 5211 bp, Homo sapiens
agccagtcctcccgtcttgcgccgcggccgcgagatccgtgtgtctcccaagatggtggcgctgggctcggggtgactacaggagacgacggggccttttcccttcgccaggacccgacacaccaggcttcgctcgctcgcgcacccctccgccgcgtagccatccgccagcgcgggcgcccgccatccgccgcctacttacgcttcacctctgccgacccggcgcgctcggctgcgggcggcggcgcctccttcggctcctcctcggaatagctcgcggcctgtagcccctggcaggagggcccctcagccccccggtgtggacaggcagcggcggctggcgacgaacgccgggatttcggcggccccggcgctccctttcccggcctcgttttccggataaggaagcgcgggtcccgcatgagccccggcggtggcggcagcgaaagagaacgaggcggtggcgggcggaggcggcgggcgagggcgactacgaccagtgaggcggccgccgcagcccaggcgcgggggcgacgacaggttaaaaatctgtaagagcctgattttagaattcaccagctcctcagaagtttggcgaaatatgagttattaagcctacgctcagatcaaggtagcagctagactggtgtgacaacctgtttttaatcagtgactcaaagctgtgatcaccctgatgtcaccgaatggccacagcttgtaaaagatcaggagaacctcagtctgacgacattgaagctagccgaatgaagcgagcagctgcaaagcatctaatagaacgctactaccaccagttaactgagggctgtggaaatgaagcctgcacgaatgagttttgtgcttcctgtccaacttttcttcgtatggataataatgcagcagctattaaagccctcgagctttataagattaatgcaaaactctgtgatcctcatccctccaagaaaggagcaagctcagcttaccttgagaactcgaaaggtgcccccaacaactcctgctctgagataaaaatgaacaagaaaggcgctagaattgattttaaagatgtgacttacttaacagaagagaaggtatatgaaattcttgaattatgtagagaaagagaggattattcccctttaatccgtgttattggaagagttttttctagtgctgaggcattggtacagagcttccggaaagttaaacaacacaccaaggaagaactgaaatctcttcaagcaaaagatgaagacaaagatgaagatgaaaaggaaaaagctgcatgttctgctgctgctatggaagaagactcagaagcatcttcctcaaggataggtgatagctcacagggagacaacaatttgcaaaaattaggccctgatgatgtgtctgtggatattgatgccattagaagggtctacaccagattgctctctaatgaaaaaattgaaactgcctttctcaatgcacttgtatatttgtcacctaacgtggaatgtgacttgacgtatcacaatgtatactctcgagatcctaattatctgaatttgttcattatcgtaatggagaatagaaatctccacagtcctgaatatctggaaatggctttgccattattttgcaaagcgatgagcaagctaccccttgcagcccaaggaaaactgatcagactgtggtctaaatacaatgcagaccagattcggagaatgatggagacatttcagcaacttattacttataaagtcataagcaatgaatttaacagtcgaaatctagtgaatgatgatgatgccattgttgctgcttcgaagtgcttgaaaatggtttactatgcaaatgtagtgggaggggaagtggacacaaatcacaatgaagaagatgatgaagagcccatccctgagtccagcgagctgacacttcaggaacttttgggagaagaaagaagaaacaagaaaggtcctcgagtggaccccctggaaactgaacttggtgttaaaaccctggattgtcgaaaaccacttatcccttttgaagagtttattaatgaaccactgaatgaggttctagaaatggataaagattatacttttttcaaagtagaaacagagaacaaattctcttttatgacatgtccctttatattgaatgctgtcacaaagaatttgggattatattatgacaatagaattcgcatgtacagtgaacgaagaatcactgttctctacagcttagttcaaggacagcagttgaatccatatttgagactcaaagttagacgtgaccatatcatagatgatgcacttgtccggctagagatgatcgctatggaaaatcctgcagacttgaagaagcagttgtatgtggaatttgaaggagaacaaggagttgatgagggaggtgtttccaaagaattttttcagctggttgtggaggaaatcttcaatccagatattggtatgttcacatacgatgaatctacaaaattgttttggtttaatccatcttcttttgaaactgagggtcagtttactctgattggcatagtactgggtctggctatttacaataactgtatactggatgtacattttcccatggttgtctacaggaagctaatggggaaaaaaggaacttttcgtgacttgggagactctcacccagttctatatcagagtttaaaagatttattggagtatgaagggaatgtggaagatgacatgatgatcactttccagatatcacagacagatctttttggtaacccaatgatgtatgatctaaaggaaaatggtgataaaattccaattacaaatgaaaacaggaaggaatttgtcaatctttattctgactacattctcaataaatcagtagaaaaacagttcaaggcttttcggagaggttttcatatggtgaccaatgaatctcccttaaagtacttattcagaccagaagaaattgaattgcttatatgtggaagccggaatctagatttccaagcactagaagaaactacagaatatgacggtggctataccagggactctgttctgattagggagttctgggaaatcgttcattcatttacagatgaacagaaaagactcttcttgcagtttacaacgggcacagacagagcacctgtgggaggactaggaaaattaaagatgattatagccaaaaatggcccagacacagaaaggttacctacatctcatacttgctttaatgtgcttttacttccggaatactcaagcaaagaaaaacttaaagagagattgttgaaggccatcacgtatgccaaaggatttggcatgctgtaaaacaaaacaaaacaaaataaaacaaaaaaaaggaaggaaaaaaaaagaaaaaatttaaaaaattttaaaaatataacgagggataaatttttggtggtgatagtgtcccagtacaaaaaggctgtaagatagtcaaccacagtagtcacctatgtctgtgcctcccttctttattggggacatgtgggctggaacagcagatttcagctacatatatgaacaaatcctttattattattataattatttttttgcgtgaaagtgttacatattctttcacttgtatgtacagagaggtttttctgaatatttattttaagggttaaatcacttttgcttgtgtttattactgcttgaggttgagccttttgagtatttaaaaaatatataccaacagaactactctcccaaggaaaatattgccaccatttgtagaccacgtaaccttcaagtatgtgctacttttttgtccctgtatctaactcaaatcaggaactgtattttttttaatgatttgcttttgaaacttgaagtcttgaaaacagtgtgatgcaattactgctgttctagcccccaaagagttttctgtgcaaaatcttgagaatcaatcaataaagaaagatggaaggaagggagaaattggaatgttttaactgcagccctcagaactttagtaacagcacaacaaattaaaaacaaaaacaactcatgccacagtatgtcgtcttcatgtgtcttgcaatgaactgtttcagtagccaatcctctttcttagtatatgaaaggacagggatttttgttcttgttgttctcgttgttgttttaagtttactggggaaagtgcatttggccaaatgaaatggtagtcaagcctattgcaacaaagttaggaagtttgttgtttgtttattataaacaaaaagcatgtgaaagtgcacttaagatagagtttttattaattacttacttattacctagattttaaatagacaatccaaagtctccccttcgtgttgccatcatcttgttgaatcagccattttatcgaggcacgtgatcagtgttgcaacataatgaaaaagatggctactgtgccttgtgttacttaatcatacagtaagctgacctggaaatgaatgaaactattactcctaagaattacattgtatagccccacagattaaatttaattaattaattcaaaacatgttaaacgttactttcatgtactatggaaaagtacaagtaggtttacattactgatttccagaagtaagtagtttcccctttcctagtcttctgtgtatgtgatgttgttaatttcttttattgcattataaaataaaaggattatgtatttttaactaaggtgagacattgatatatccttttgctacaagctatagctaatgtgctgagcttgtgccttggtgattgattgattgattgactgattgttttaactgattactgtagatcaacctgatgatttgtttgtttgaaattggcaggaaaaatgcagctttcaaatcattggggggagaaaaaggatgtctttcaggattattttaattaatttttttcataattgagacagaactgtttgttatgtaccataatgctaaataaaactgtggcacttttcaccataatttaatttagtggaaaaagaagacaatgctttccatattgtgataaggtaacatggggtttttctgggccagcctttagaacactgttagggtacatacgctaccttgatgaaagggaccttcgtgcaactgtagtcatcttaaaggcttctcatccactgtgcttcttaatgtgtaattaaagtgaggagaaattaaatactctgagggcgttttatataataaattcgtgaaga

SEQ ID NO: 6 - DNA, 4491 bp, Homo sapiens
acagatcaggagaacctcagtctgacgacattgaagctagccgaatgaagcgagcagctgcaaagcatctaatagaacgctactaccaccagttaactgagggctgtggaaatgaagcctgcacgaatgagttttgtgcttcctgtccaacttttcttcgtatggataataatgcagcagctattaaagccctcgagctttataagattaatgcaaaactctgtgatcctcatccctccaagaaaggagcaagctcagcttaccttgagaactcgaaaggtgcccccaacaactcctgctctgagataaaaatgaacaagaaaggcgctagaattgattttaaagatgtgacttacttaacagaagagaaggtatatgaaattcttgaattatgtagagaaagagaggattattcccctttaatccgtgttattggaagagttttttctagtgctgaggcattggtacagagcttccggaaagttaaacaacacaccaaggaagaactgaaatctcttcaagcaaaagatgaagacaaagatgaagatgaaaaggaaaaagctgcatgttctgctgctgctatggaagaagactcagaagcatcttcctcaaggataggtgatagctcacagggagacaacaatttgcaaaaattaggccctgatgatgtgtctgtggatattgatgccattagaagggtctacaccagattgctctctaatgaaaaaattgaaactgcctttctcaatgcacttgtatatttgtcacctaacgtggaatgtgacttgacgtatcacaatgtatactctcgagatcctaattatctgaatttgttcattatcgtaatggagaatagaaatctccacagtcctgaatatctggaaatggctttgccattattttgcaaagcgatgagcaagctaccccttgcagcccaaggaaaactgatcagactgtggtctaaatacaatgcagaccagattcggagaatgatggagacatttcagcaacttattacttataaagtcataagcaatgaatttaacagtcgaaatctagtgaatgatgatgatgccattgttgctgcttcgaagtgcttgaaaatggtttactatgcaaatgtagtgggaggggaagtggacacaaatcacaatgaagaagatgatgaagagcccatccctgagtccagcgagctgacacttcaggaacttttgggagaagaaagaagaaacaagaaaggtcctcgagtggaccccctggaaactgaacttggtgttaaaaccctggattgtcgaaaaccacttatcccttttgaagagtttattaatgaaccactgaatgaggttctagaaatggataaagattatacttttttcaaagtagaaacagagaacaaattctcttttatgacatgtccctttatattgaatgctgtcacaaagaatttgggattatattatgacaatagaattcgcatgtacagtgaacgaagaatcactgttctctacagcttagttcaaggacagcagttgaatccatatttgagactcaaagttagacgtgaccatatcatagatgatgcacttgtccggctagagatgatcgctatggaaaatcctgcagacttgaagaagcagttgtatgtggaatttgaaggagaacaaggagttgatgagggaggtgtttccaaagaattttttcagctggttgtggaggaaatcttcaatccagatattggtatgttcacatacgatgaatctacaaaattgttttggtttaatccatcttcttttgaaactgagggtcagtttactctgattggcatagtactgggtctggctatttacaataactgtatactggatgtacattttcccatggttgtctacaggaagctaatggggaaaaaaggaacttttcgtgacttgggagactctcacccagttctatatcagagtttaaaagatttattggagtatgaagggaatgtggaagatgacatgatgatcactttccagatatcacagacagatctttttggtaacccaatgatgtatgatctaaaggaaaatggtgataaaattccaattacaaatgaaaacaggaaggaatttgtcaatctttattctgactacattctcaataaatcagtagaaaaacagttcaaggcttttcggagaggttttcatatggtgaccaatgaatctcccttaaagtacttattcagaccagaagaaattgaattgcttatatgtggaagccggaatctagatttccaagcactagaagaaactacagaatatgacggtggctataccagggactctgttctgattagggagttctgggaaatcgttcattcatttacagatgaacagaaaagactcttcttgcagtttacaacgggcacagacagagcacctgtgggaggactaggaaaattaaagatgattatagccaaaaatggcccagacacagaaaggttacctacatctcatacttgctttaatgtgcttttacttccggaatactcaagcaaagaaaaacttaaagagagattgttgaaggccatcacgtatgccaaaggatttggcatgctgtaaaacaaaacaaaacaaaataaaacaaaaaaaaggaaggaaaaaaaaagaaaaaatttaaaaaattttaaaaatataacgagggataaatttttggtggtgatagtgtcccagtacaaaaaggctgtaagatagtcaaccacagtagtcacctatgtctgtgcctcccttctttattggggacatgtgggctggaacagcagatttcagctacatatatgaacaaatcctttattattattataattatttttttgcgtgaaagtgttacatattctttcacttgtatgtacagagaggtttttctgaatatttattttaagggttaaatcacttttgcttgtgtttattactgcttgaggttgagccttttgagtatttaaaaaatatataccaacagaactactctcccaaggaaaatattgccaccatttgtagaccacgtaaccttcaagtatgtgctacttttttgtccctgtatctaactcaaatcaggaactgtattttttttaatgatttgcttttgaaacttgaagtcttgaaaacagtgtgatgcaattactgctgttctagcccccaaagagttttctgtgcaaaatcttgagaatcaatcaataaagaaagatggaaggaagggagaaattggaatgttttaactgcagccctcagaactttagtaacagcacaacaaattaaaaacaaaaacaactcatgccacagtatgtcgtcttcatgtgtcttgcaatgaactgtttcagtagccaatcctctttcttagtatatgaaaggacagggatttttgttcttgttgttctcgttgttgttttaagtttactggggaaagtgcatttggccaaatgaaatggtagtcaagcctattgcaacaaagttaggaagtttgttgtttgtttattataaacaaaaagcatgtgaaagtgcacttaagatagagtttttattaattacttacttattacctagattttaaatagacaatccaaagtctccccttcgtgttgccatcatcttgttgaatcagccattttatcgaggcacgtgatcagtgttgcaacataatgaaaaagatggctactgtgccttgtgttacttaatcatacagtaagctgacctggaaatgaatgaaactattactcctaagaattacattgtatagccccacagattaaatttaattaattaattcaaaacatgttaaacgttactttcatgtactatggaaaagtacaagtaggtttacattactgatttccagaagtaagtagtttcccctttcctagtcttctgtgtatgtgatgttgttaatttcttttattgcattataaaataaaaggattatgtatttttaactaaggtgagacattgatatatccttttgctacaagctatagctaatgtgctgagcttgtgccttggtgattgattgattgattgactgattgttttaactgattactgtagatcaacctgatgatttgtttgtttgaaattggcaggaaaaatgcagctttcaaatcattggggggagaaaaaggatgtctttcaggattattttaattaatttttttcataattgagacagaactgtttgttatgtaccataatgctaaataaaactgtggcacttttcaccataatttaatttagtggaaaaagaagacaatgctttccatattgtgataaggtaacatggggtttttctgggccagcctttagaacactgttagggtacatacgctaccttgatgaaagggaccttcgtgcaactgtagtcatcttaaaggcttctcatccactgtgcttcttaatgtgtaattaaagtgaggagaaattaaatactctgagggcgttttatataataaattcgtgaaga

SEQ ID NO: 7 - protein, 849 aa, Mus musculus
MKRAAAKHLIERYYHQLTEGCGNEACTNEFCASCPTFLRMDNNAAAIKALELYKINAKLCDPHPSKKGASSAYLENSKGASNNSEIKMNKKEGKDFKDVIYLTEEKVYEIYEFCRESEDYSPLIRVIGRIFSSAEALVLSFRKVKQHTKEELKSLQEKDEDKDEDEKEKAACSAAAMEEDSEASSSRMGDSSQGDNNVQKLGPDDVTVDIDAIRRVYSSLLANEKLETAFLNALVYLSPNVECDLTYHNVYTRDPNYLNLFIIVMENSNLHSPEYLEMALPLFCKAMCKLPLEAQGKLIRLWSKYSADQIRRMMETFQQLITYKVISNEFNSRNLVNDDDAIVAASKCLKMVYYANVVGGDVDTNHNEEDDEEPIPESSELTLQELLGDERRNKKGPRVDPLETELGVKTLDCRKPLISFEEFINEPLNDVLEMDKDYTFFKVETENKFSFMTCPFILNAVTKNLGLYYDNRIRMYSERRITVLYSLVQGQQLNPYLRLKVRRDHIIDDALVRLEMIAMENPADLKKQLYVEFEGEQGVDEGGVSKEFFQLVVEEIFNPDIGMFTYDEATKLFWFNPSSFETEGQFTLIGIVLGLAIYNNCILDVHFPMVVYRKLMGKKGTFRDLGDSHPVLYQSLKDLLEYEGSVEDDMMITFQISQTDLFGNPMMYDLKENGDKIPITNENRKEFVNLYSDYILNKSVEKQFKAFRRGFHMVTNESPLKYLFRPEEIELLICGSRNLDFQALEETTEYDGGYTRESVVIREFWEIVHSFTDEQKRLFLQFTTGTDRAPVGGLGKLKMIIAKNGPDTERLPTSHTCFNVLLLPEYSSKEKLKERLLKAITYAKGFGML

SEQ ID NO: 8 - protein, 870 aa, Mus musculus
MATACKRSPGESQSEDIEASRMKRAAAKHLIERYYHQLTEGCGNEACTNEFCASCPTFLRMDNNAAAIKALELYKINAKLCDPHPSKKGASSAYLENSKGASNNSEIKMNKKEGKDFKDVIYLTEEKVYEIYEFCRESEDYSPLIRVIGRIFSSAEALVLSFRKVKQHTKEELKSLQEKDEDKDEDEKEKAACSAAAMEEDSEASSSRMGDSSQGDNNVQKLGPDDVTVDIDAIRRVYSSLLANEKLETAFLNALVYLSPNVECDLTYHNVYTRDPNYLNLFIIVMENSNLHSPEYLEMALPLFCKAMCKLPLEAQGKLIRLWSKYSADQIRRMMETFQQLITYKVISNEFNSRNLVNDDDAIVAASKCLKMVYYANVVGGDVDTNHNEEDDEEPIPESSELTLQELLGDERRNKKGPRVDPLETELGVKTLDCRKPLISFEEFINEPLNDVLEMDKDYTFFKVETENKFSFMTCPFILNAVTKNLGLYYDNRIRMYSERRITVLYSLVQGQQLNPYLRLKVRRDHIIDDALVRLEMIAMENPADLKKQLYVEFEGEQGVDEGGVSKEFFQLVVEEIFNPDIGMFTYDEATKLFWFNPSSFETEGQFTLIGIVLGLAIYNNCILDVHFPMVVYRKLMGKKGTFRDLGDSHPVLYQSLKDLLEYEGSVEDDMMITFQISQTDLFGNPMMYDLKENGDKIPITNENRKEFVNLYSDYILNKSVEKQFKAFRRGFHMVTNESPLKYLFRPEEIELLICGSRNLDFQALEETTEYDGGYTRESVVIREFWEIVHSFTDEQKRLFLQFTTGTDRAPVGGLGKLKMIIAKNGPDTERLPTSHTCFNVLLLPEYSSKEKLKERLLKAITYAKGFGML

SEQ ID NO: 9 - protein, 762 aa, Mus musculus
MKRAAAKHLIERYYHQLTEGCGNEACTNEFCASCPTFLRMDNNAAAIKALELYKINAKLCDPHPSKKGASSAYLENSKGASNNSEIKMNKKEGKDFKDVIYLTEEKVYEIYEFCRESEDYSPLIRVIGRIFSSAEALVLSFRKVKQHTKEELKSLQEKDEDKDEDEKEKAACSAAAMEEDSEASSSRMGDSSQGDNNVQKLGPDDVTVDIDAIRRVYSSLLANEKLETAFLNALVYLSPNVECDLTYHNVYTRDPNYLNLFIIVMENSNLHSPEYLEMALPLFCKAMCKLPLEAQGKLIRLWSKYSADQIRRMMETFQQLITYKVISNEFNSRNLVNDDDAIVAASKCLKMVYYANVVGGDVDTNHNEEDDEEPIPESSELTLQELLGDERRNKKGPRVDPLETELGVKTLDCRKPLISFEEFINEPLNDVLEMDKDYTFFKVETENKFSFMTCPFILNAVTKNLGLYYDNRIRMYSERRITVLYSLVQGQQLNPYLRLKVRRDHIIDDALVRLEMIAMENPADLKKQLYVEFEGEQGVDEGGVSKEFFQLVVEEIFNPDIGMFTYDEATKLFWFNPSSFETEGQFTLIGIVLGLAIYNNCILDVHFPMVVYRKLMGKKGTFRDLGDSHPVLYQSLKDLLEYEGSVEDDMMITFQISQTDLFGNPMMYDLKENGDKIPITNENRKEFVNLYSDYILNKSVEKQFKAFRRGFHMVTNESPLKYLFRPEEIELLICGSRNLDFQALEETTEYDGGYTRESVVIR

SEQ ID NO: 10 - protein, 875 aa, Homo sapiens
MEKLHQCYWKSGEPQSDDIEASRMKRAAAKHLIERYYHQLTEGCGNEACTNEFCASCPTFLRMDNNAAAIKALELYKINAKLCDPHPSKKGASSAYLENSKGAPNNSCSEIKMNKKGARIDFKDVTYLTEEKVYEILELCREREDYSPLIRVIGRVFSSAEALVQSFRKVKQHTKEELKSLQAKDEDKDEDEKEKAACSAAAMEEDSEASSSRIGDSSQGDNNLQKLGPDDVSVDIDAIRRVYTRLLSNEKIETAFLNALVYLSPNVECDLTYHNVYSRDPNYLNLFIIVMENRNLHSPEYLEMALPLFCKAMSKLPLAAQGKLIRLWSKYNADQIRRMMETFQQLITYKVISNEFNSRNLVNDDDAIVAASKCLKMVYYANVVGGEVDTNHNEEDDEEPIPESSELTLQELLGEERRNKKGPRVDPLETELGVKTLDCRKPLIPFEEFINEPLNEVLEMDKDYTFFKVETENKFSFMTCPFILNAVTKNLGLYYDNRIRMYSERRITVLYSLVQGQQLNPYLRLKVRRDHIIDDALVRLEMIAMENPADLKKQLYVEFEGEQGVDEGGVSKEFFQLVVEEIFNPDIGMFTYDESTKLFWFNPSSFETEGQFTLIGIVLGLAIYNNCILDVHFPMVVYRKLMGKKGTFRDLGDSHPVLYQSLKDLLEYEGNVEDDMMITFQISQTDLFGNPMMYDLKENGDKIPITNENRKEFVNLYSDYILNKSVEKQFKAFRRGFHMVTNESPLKYLFRPEEIELLICGSRNLDFQALEETTEYDGGYTRDSVLIREFWEIVHSFTDEQKRLFLQFTTGTDRAPVGGLGKLKMIIAKNGPDTERLPTSHTCFNVLLLPEYSSKEKLKERLLKAITYAKGFGML

SEQ ID NO: 11 - protein, 872 aa, Homo sapiens
MATACKRSGEPQSDDIEASRMKRAAAKHLIERYYHQLTEGCGNEACTNEFCASCPTFLRMDNNAAAIKALELYKINAKLCDPHPSKKGASSAYLENSKGAPNNSCSEIKMNKKGARIDFKDVTYLTEEKVYEILELCREREDYSPLIRVIGRVFSSAEALVQSFRKVKQHTKEELKSLQAKDEDKDEDEKEKAACSAAAMEEDSEASSSRIGDSSQGDNNLQKLGPDDVSVDIDAIRRVYTRLLSNEKIETAFLNALVYLSPNVECDLTYHNVYSRDPNYLNLFIIVMENRNLHSPEYLEMALPLFCKAMSKLPLAAQGKLIRLWSKYNADQIRRMMETFQQLITYKVISNEFNSRNLVNDDDAIVAASKCLKMVYYANVVGGEVDTNHNEEDDEEPIPESSELTLQELLGEERRNKKGPRVDPLETELGVKTLDCRKPLIPFEEFINEPLNEVLEMDKDYTFFKVETENKFSFMTCPFILNAVTKNLGLYYDNRIRMYSERRITVLYSLVQGQQLNPYLRLKVRRDHIIDDALVRLEMIAMENPADLKKQLYVEFEGEQGVDEGGVSKEFFQLVVEEIFNPDIGMFTYDESTKLFWFNPSSFETEGQFTLIGIVLGLAIYNNCILDVHFPMVVYRKLMGKKGTFRDLGDSHPVLYQSLKDLLEYEGNVEDDMMITFQISQTDLFGNPMMYDLKENGDKIPITNENRKEFVNLYSDYILNKSVEKQFKAFRRGFHMVTNESPLKYLFRPEEIELLICGSRNLDFQALEETTEYDGGYTRDSVLIREFWEIVHSFTDEQKRLFLQFTTGTDRAPVGGLGKLKMIIAKNGPDTERLPTSHTCFNVLLLPEYSSKEKLKERLLKAITYAKGFGML

SEQ ID NO: 12 - protein, 852 aa, Homo sapiens
MKRAAAKHLIERYYHQLTEGCGNEACTNEFCASCPTFLRMDNNAAAIKALELYKINAKLCDPHPSKKGASSAYLENSKGAPNNSCSEIKMNKKGARIDFKDVTYLTEEKVYEILELCREREDYSPLIRVIGRVFSSAEALVQSFRKVKQHTKEELKSLQAKDEDKDEDEKEKAACSAAAMEEDSEASSSRIGDSSQGDNNLQKLGPDDVSVDIDAIRRVYTRLLSNEKIETAFLNALVYLSPNVECDLTYHNVYSRDPNYLNLFIIVMENRNLHSPEYLEMALPLFCKAMSKLPLAAQGKLIRLWSKYNADQIRRMMETFQQLITYKVISNEFNSRNLVNDDDAIVAASKCLKMVYYANVVGGEVDTNHNEEDDEEPIPESSELTLQELLGEERRNKKGPRVDPLETELGVKTLDCRKPLIPFEEFINEPLNEVLEMDKDYTFFKVETENKFSFMTCPFILNAVTKNLGLYYDNRIRMYSERRITVLYSLVQGQQLNPYLRLKVRRDHIIDDALVRLEMIAMENPADLKKQLYVEFEGEQGVDEGGVSKEFFQLVVEEIFNPDIGMFTYDESTKLFWFNPSSFETEGQFTLIGIVLGLAIYNNCILDVHFPMVVYRKLMGKKGTFRDLGDSHPVLYQSLKDLLEYEGNVEDDMMITFQISQTDLFGNPMMYDLKENGDKIPITNENRKEFVNLYSDYILNKSVEKQFKAFRRGFHMVTNESPLKYLFRPEEIELLICGSRNLDFQALEETTEYDGGYTRDSVLIREFWEIVHSFTDEQKRLFLQFTTGTDRAPVGGLGKLKMIIAKNGPDTERLPTSHTCFNVLLLPEYSSKEKLKERLLKAITYAKGFGML

SEQ ID NO: 13 - DNA, 96 bp, Mus musculus
aaaggatttggcatgctggactacaaagaccatgacggtgattataaagatcatgatatcgactacaaagatgacgacgataaatagtaatgtagg

SEQ ID NO: 14 - protein, 28 aa, Mus musculus
KGFGMLDYKDHDGDYKDHDIDYKDDDDK